(19) United States
(12) Patent Application Publication
(10) Pub. No.: US 2011/0033451 A1
(43) Pub. Date: Feb. 10, 2011

(54) INTERLEUKIN-17F ANTIBODIES AND OTHER IL-17F SIGNALING ANTAGONISTS AND USES THEREFOR

(75) Inventors: Beatriz M. Carreno, Clayton, MO (US); Mary Collins, Natick, MA (US); Jill F. Wright, Ashland, MA (US); Neil M. Wolfman, Dover, MA (US); Maya Arai, Brookline, MA (US); Kenneth Jacobs, Newton, MA (US); Zhijian Lu, Bedford, MA (US); Yongjing Guo, Chestnut Hill, MA (US); Yongchang Qiu, Acton, MA (US)

Correspondence Address: WYETH LLC;PATENT LAW GROUP, 5 GIRALDA FARMS, MADISON, NJ, 07940, US

(73) Assignee: Wyeth LLC, Madison, NJ (US)

(21) Appl. No.: 12/827,199

(22) Filed: Jun. 30, 2010

Related U.S. Application Data
(63) Continuation of application No. 12/196,117, filed on Aug. 21, 2008, now abandoned.
Application No. 12/196,117 is a division of application No. 11/353,161, filed on Feb. 14, 2006, now abandoned.
(60) Provisional application No. 60/653,260, filed on Feb. 14, 2005.
(60) Provisional application No. 60/667,492, filed on Apr. 1, 2005.

(51) Int. Cl. A61K 39/395; A61K 33/24; A61K 38/02; A61K 31/7088; A61P 11/06; A61P 29/00; A61P 19/02; A61P 17/06

(52) U.S. Cl. 424/133.1; 424/130.1; 424/649; 514/1.1; 514/44 A

(57) ABSTRACT

The present invention provides isolated and purified polynucleotides and polypeptides related to the IL-17F signaling pathway. The invention also provides antibodies to IL-17F homodimers and IL-17A/IL-17F heterodimers, and methods of isolating and purifying members of the IL-17 family, including IL-17A/IL-17F heterodimers, from a natural source. The present invention also is directed to novel methods for diagnosing, prognosing, monitoring the progress of, and treating and/or preventing disorders related to IL-17F signaling, i.e., IL-17F-associated disorders, including, but not limited to, inflammatory disorders, such as autoimmune diseases (e.g., arthritis (including rheumatoid arthritis), psoriasis, systemic lupus erythematosus, and multiple sclerosis), respiratory diseases (e.g., COPD, cystic fibrosis, asthma, allergy), transplant rejection (including solid organ transplant rejection), and inflammatory bowel diseases or disorders (IBDs, e.g., ulcerative colitis, Crohn's disease). The present invention is further directed to novel therapeutics and therapeutic targets, and to methods of screening and assessing test compounds for the intervention (treatment) and prevention of disorders related to IL-17F signaling.

### RELATED APPLICATIONS

This application is a continuation of U.S. application Ser. No. 12/196,117, filed Aug. 21, 2008, which is a divisional of U.S. application Ser. No. 11/353,161, filed Feb. 14, 2006, which claims priority to U.S. Provisional Patent Application No. 60/653,260, filed Feb. 14, 2005; and U.S. Provisional Patent Application No. 60/667,492, filed Apr. 1, 2005. The contents of the prior applications are hereby incorporated by reference herein in their entireties.

### BACKGROUND OF THE INVENTION

1. Field of the Invention

This invention relates to antibodies, e.g., intact antibodies and antigen-binding fragments thereof, and other IL-17F signaling antagonists, e.g., soluble IL-17F receptor(s), that interfere with interleukin-17F (IL-17F) signaling, in particular, human IL-17F, and their uses in regulating IL-17F-associated activities. The antibodies and related IL-17F molecules disclosed herein are useful in diagnosing, prognosing, monitoring, preventing, and/or treating IL-17F-associated disorders, e.g., inflammatory disorders (e.g., autoimmune diseases (e.g., arthritis (including rheumatoid arthritis), psoriasis, systemic lupus erythematosus (SLE), multiple sclerosis), respiratory diseases (e.g., COPD, cystic fibrosis, asthma, allergy), transplant rejection (including solid organ transplant rejection), and inflammatory bowel diseases or disorders (IBDs, e.g., ulcerative colitis, Crohn's disease)).

2. Related Background Art

Cytokines are secreted soluble proteins with pleiotropic activities involved in immune and inflammatory responses, e.g., cytokines may cause differentiation, recruitment, or other physiological responses, e.g., secretion of proteins characteristic of inflammation, by target cells. Cytokines bind to specific cell surface receptors, triggering signal transduction pathways that lead to cell activation, proliferation, and differentiation. One such cytokine, interleukin-17 (IL-17), originally named CTLA-8, was isolated and cloned from murine hybridomas and shown to have homology to open reading frame 13 of the T lymphotropic Herpesvirus saimiri (Rouvier et al. (1993) J. Immunol. 150:5445-56; Yao et al. (1996) Gene 168:223-25; Golstein et al., published International Patent Application No. WO95/01826). Since then, five related cytokines that share 20-50% homology to IL-17 have been identified (see Moseley et al. (2003) Cytokine & Growth Factor Reviews 14: 155-74). To indicate IL-17 as the founding member of the IL-17 cytokine family, it has been designated IL-17A (Moseley, supra); the other members have been designated IL-17B, IL-17C, IL-17D, IL-17E, and IL-17F. IL-17 cytokine family members share conserved cysteine residues. Of interest are IL-17A and particularly IL-17F, which share 50% identity; both cytokines are induced by IL-23, coexpressed by T cells, and considered potential targets for T cell-mediated autoimmune diseases. Similar to IL-17A, the conserved cysteine residues in IL-17F exhibit features of a classic cysteine knot motif found in bone morphogenetic proteins (BMPs), transforming growth factor-beta (TGF-β), nerve growth factor (NGF) and platelet-derived factor BB (PDGF-BB) (Hymowitz et al. (2001) EMBO J. 20:5332-41; McDonald et al. (1993) Cell 73:421-24).

IL-17F is a 17 kD secreted protein that was cloned from an activated human PBMC library (SST) (U.S. Pat. Nos. 6,043,344 and 6,074,849). It forms a 30-35 kD disulfide-linked homodimer (Hymowitz, supra) and, similar to IL-17A, is expressed primarily by activated T cells (Moseley, supra). However, expression of IL-17F by activated monocytes, activated basophils and mast cells has also been shown (Kawaguchi et al. (2002) J. Immunol. 167:4430-35). IL-17F induces the expression of many cytokines and chemokines by macrophages, endothelial cells, epithelial cells, and fibroblasts (Moseley, supra).

IL-17F plays a role in inflammatory responses, in part, by inducing the production of inflammatory cytokines and neutrophilia. It is associated with the development of several autoimmune diseases, e.g., arthritis (including rheumatoid and Lyme arthritis), systemic lupus erythematosus (SLE), and asthma (Bettelli and Kuchroo (2005) J. Exp. Med. 201:169-71). For example, it has recently been shown that IL-23 is essential for the expansion of a T cell population which is characterized by, inter alia, production of IL-17F, that passive transfer of this T cell population is essential for the establishment of organ-specific inflammation associated with central nervous system autoimmunity (Langrish et al. (2005) J. Exp. Med. 201:233-40), and that IL-17-deficient mice are resistant to experimental autoimmune encephalomyelitis (EAE; an animal model for multiple sclerosis) (Nakae et al. (2003) J. Immunol. 171:6173-77). IL-17F is unique among known inflammatory cytokines in that it increases proteoglycan breakdown and decreases proteoglycan synthesis by articular cartilage (Hymowitz, supra). Additionally, increased expression of IL-17F has been demonstrated in bronchoalveolar lavages (BALs) taken from patients suffering with asthma after allergen challenge compared to BALs taken from these patients as controls (Kawaguchi, supra). Also, IL-17F mRNA expression is increased in patients with ulcerative colitis and Crohn's disease (Gurney et al. (2003) GTCBIO Conf: Cytokines and Beyond). These observations suggest that blockade of IL-17F signaling will reduce proinflammatory cytokine production and decrease bone erosion. Consequently, the IL-17F signaling pathway is an attractive target for treating and/or preventing inflammatory diseases, e.g., in which recruited neutrophils are critical mediators of tissue injury, e.g., during the development of autoimmune diseases (e.g., arthritis (including rheumatoid arthritis), psoriasis, systemic lupus erythematosus, multiple sclerosis), respiratory diseases (e.g., COPD, cystic fibrosis, asthma, allergy), transplant rejection (including solid organ transplant rejection), and inflammatory bowel disorders or diseases (IBDs, e.g., ulcerative colitis, Crohn's disease).

Currently, not much is known about the receptors for members of the IL-17 family. It has been shown that IL-17R, the receptor for IL-17A, is expressed in all tissues examined to date, and that binding of IL-17R by IL-17A generally results in the induction of proinflammatory cytokines through activation of NF-κB (Moseley, supra). Four additional receptors that share partial sequence homology to IL-17R have been identified: 1) IL-17RH1 (also called IL-17RB), 2) IL-17-receptor like protein (also called IL-17RL or IL-17RC), 3) IL-17RD (also called SEF or IL-17RLM), and 4) IL-17RE (Moseley, supra). Of these four additional receptors, only IL-17RH1 has been shown to bind to IL-17 cytokines, namely IL-17B and IL-17E; however, the function of IL-17B and IL-17E binding to IL-17RH1 has not been shown (Shi et al. (2000) J. Biol. Chem. 275:19167-76; Lee et al. (2001) J. Biol. Chem. 276:1660-64). To date, the receptor(s) for IL-17F has not been reported. Thus, IL-17F signaling has not been able to be targeted for the prevention and/or treatment of diseases, although it may play an important role in the homeostasis of tissues (e.g., joint tissues) and the progression of various diseases (e.g., arthritis, asthma, allergy, COPD, cystic fibrosis, ulcerative colitis, Crohn's disease, etc.). The present invention solves this problem by identifying and targeting key players involved in the signal transduction pathway of IL-17F protein.

### SUMMARY OF THE INVENTION

An object of the invention is to identify components of the IL-17F signaling pathway, e.g., IL-17F and its receptor, and to target these components in methods of treating disorders related to IL-17F signaling. Such IL-17F-associated disorders and disorders related to increased IL-17F signaling include, but are not limited to, inflammatory disorders, e.g., autoimmune diseases (e.g., arthritis (including rheumatoid arthritis), psoriasis, systemic lupus erythematosus, multiple sclerosis), respiratory diseases (e.g., COPD, cystic fibrosis, asthma, allergy), transplant rejection (including solid organ transplant rejection), and inflammatory bowel diseases (e.g., ulcerative colitis, Crohn's disease).

As such, the research underlying the present invention provides evidence that IL-17F mediates proteoglycan destruction and inflammatory responses through its binding to IL-17R and/or IL-17RC. The determination of IL-17R and IL-17RC as receptors for IL-17F exposes these molecules as targets for the treatment of disorders related to IL-17F signaling.

Provided herein are IL-17F signaling antagonists, including, but not limited to, IL-17F inhibitory polynucleotides, IL-17R inhibitory polynucleotides, IL-17RC inhibitory polynucleotides, soluble polypeptides comprising IL-17R or IL-17F-binding fragments thereof, soluble polypeptides comprising IL-17RC or IL-17F-binding fragments thereof, inhibitory anti-IL-17F antibodies, inhibitory anti-IL-17R antibodies, inhibitory anti-IL-17RC antibodies, and antagonistic small molecules. Preferred examples of IL-17F signaling antagonists include siRNAs directed to IL-17R and IL-17RC, soluble fusion proteins comprising IL-17R and IL-17RC (or IL-17F-binding fragments thereof), and inhibitory (i.e., antagonistic) IL-17F antibodies. In another preferred embodiment of the invention, an IL-17F signaling antagonist, e.g., siRNAs directed against IL-17R or IL-17RC, soluble fusion proteins comprising IL-17R or IL-17RC (or IL-17F binding fragments thereof), or inhibitory IL-17F antibodies, decreases IL-17F bioactivity and/or the ability of NF-κB to activate NF-κB responsive genes.

Additionally, based on structural and sequence similarity between IL-17A and IL-17F, the inventors hypothesized and demonstrated the formation of novel IL-17A/IL-17F heterodimers. In demonstrating the existence of IL-17A/IL-17F heterodimers, the inventors are the first to demonstrate that IL-21 results in the increased production of IL-17A homodimers, IL-17F homodimers, and IL-17A/IL-17F heterodimers, and suggest that effects associated with IL-21 binding to and activating IL-21R may be due, at least in part, to IL-17 signaling. The inventors are also the first to isolate IL-17A homodimers, IL-17F homodimers and IL-17A/IL-17F heterodimers from a natural source of these cytokines, e.g., activated T cells. Thus, the invention also provides methods of mitigating effects associated with IL-21 binding to and activating IL-21R, e.g., by inhibiting IL-17A and/or IL-17F signaling. Additionally, the invention provides natural (i.e., nonrecombinant) IL-17A homodimers, IL-17F homodimers, and IL-17A/IL-17F heterodimers, and methods of isolating and targeting the same, e.g., in methods of treating disorders associated with increased IL-17F signaling and/or disorders associated with IL-21 binding to and activating IL-21R. Disclosed herein additionally are recombinant IL-17A homodimers, IL-17F homodimers, and IL-17A/IL-17F heterodimers, and methods of isolating IL-17A/IL-17F heterodimers (either recombinant or natural) substantially free of IL-17A homodimers and IL-17F homodimers.

Methods that target IL-17F signaling may involve IL-17F, IL-17R and/or IL-17RC polynucleotides (including inhibitory polynucleotides such as antisense, siRNA, and aptamers), polypeptides, and fragments thereof as IL-17F signaling antagonists. Additionally, antibodies capable of inhibiting the interaction of IL-17F protein (either as an IL-17F homodimer or as an IL-17A/IL-17F heterodimer) with its receptor(s) may also be used.

The invention also relates to using the molecules disclosed herein in methods of screening test compounds capable of targeting the IL-17F signaling pathway, and diagnosing, prognosing, monitoring and/or treating disorders related to IL-17F signaling.

In one embodiment, the present invention provides a method of screening for test compounds capable of antagonizing IL-17F signaling comprising the steps of: contacting a sample containing IL-17F and IL-17R with a compound; and determining whether the interaction of IL-17F with IL-17R in the sample is decreased relative to the interaction of IL-17F with IL-17R in a sample not contacted with the compound, whereby such a decrease in the interaction of IL-17F with IL-17R in the sample contacted with the compound identifies the compound as one that inhibits the interaction of IL-17F with IL-17R and is capable of antagonizing IL-17F signaling. In another embodiment, the invention provides a similar method of screening related to IL-17RC.

In another embodiment, the invention provides a method for diagnosing a disorder related to increased IL-17F signaling in a subject comprising the steps of: detecting a test amount of an IL-17F signaling gene product in a sample from the subject; and comparing the test amount with a normal amount of the same IL-17F signaling gene product in a control sample, whereby a test amount significantly above the normal amount provides a positive indication in the diagnosis of a disorder related to increased IL-17F signaling. In another embodiment, the disorder is selected from the group consisting of autoimmune diseases, respiratory diseases, and inflammatory bowel diseases. In other embodiments, the IL-17F signaling gene product is an IL-17F gene product, an IL-17R gene product, or an IL-17RC gene product.

In another embodiment, the invention provides a method of treating a subject at risk for, or diagnosed with, a disorder related to increased IL-17F signaling comprising administering to the subject a therapeutically effective amount of an IL-17F signaling antagonist. In another embodiment, the IL-17F signaling antagonist is selected from the group consisting of IL-17F inhibitory polynucleotides, IL-17R inhibitory polynucleotides, IL-17RC inhibitory polynucleotides, soluble polypeptides comprising IL-17R or IL-17F binding fragments thereof, soluble polypeptides comprising IL-17RC or IL-17F binding fragments thereof, inhibitory anti-IL-17F antibodies, inhibitory anti-IL-17R antibodies, inhibitory IL-17RC antibodies, and antagonistic small molecules. In some embodiments, the IL-17F signaling antagonist is an IL-17R inhibitory polynucleotide or an IL-17RC inhibitory polynucleotide. In some further embodiments, the inhibitory polynucleotide is an siRNA selected from the group consisting of the nucleotide sequences set forth in SEQ ID NOs:17-32. In some embodiments, the IL-17F signaling antagonist is a soluble polypeptide comprising IL-17R or IL-17F binding fragments thereof, or comprising IL-17RC or IL-17F binding fragments thereof. In some further embodiments, the soluble polypeptide has the amino acid sequence set forth in SEQ ID NO:34 or SEQ ID NO:35. In some other embodiments, (1) the IL-17F inhibitory polynucleotide comprises the nucleotide sequence set forth in, or a nucleotide sequence complementary to the nucleotide sequence set forth in, SEQ ID NO:1 or a fragment of SEQ ID NO:1, or an RNA equivalent thereof, and wherein expression of the inhibitory polynucleotide in a cell results in the decreased expression of IL-17F; (2) the IL-17R inhibitory polynucleotide comprises the nucleotide sequence set forth in, or a nucleotide sequence complementary to the nucleotide sequence set forth in, SEQ ID NO:5 or a fragment of SEQ ID NO:5, or an RNA equivalent thereof, and wherein expression of the inhibitory polynucleotide in a cell results in the decreased expression of IL-17R; and (3) the IL-17RC inhibitory polynucleotide comprises a nucleotide sequence selected from the group consisting of the nucleotide sequences set forth in, or a nucleotide sequence complementary to a nucleotide sequence selected from the group consisting of the nucleotide sequences set forth in, SEQ ID NO:7, SEQ ID NO:9, SEQ ID NO:11, SEQ ID NO:13, and SEQ ID NO:15 or a fragment of a nucleotide sequence selected from the group consisting of the nucleotide sequences set forth in SEQ ID NO:7, SEQ ID NO:9, SEQ ID NO:11, SEQ ID NO:13, and SEQ ID NO:15, or an RNA equivalent thereof, and wherein expression of the inhibitory polynucleotide in a cell results in the decreased expression of IL-17RC. In some embodiments, the disorder related to increased IL-17F signaling is an inflammatory disorder. In some further embodiments, the inflammatory disorder is selected from the group consisting of an autoimmune disease, a respiratory disease, and an inflammatory bowel disease. In some further embodiments, the inflammatory disorder is an autoimmune disease, and the autoimmune disease is selected from the group consisting of arthritis (including rheumatoid arthritis), psoriasis, systemic lupus erythematosus, and multiple sclerosis. In some further embodiments, the inflammatory disorder is a respiratory disease, and the respiratory disease is cystic fibrosis; or the inflammatory disorder is an inflammatory bowel disease.

In another embodiment, the invention further comprises administering to the subject a therapeutically effective amount of at least one additional therapeutic agent. In another embodiment, the at least one additional therapeutic agent is selected from the group consisting of cytokine inhibitors, growth factor inhibitors, immunosuppressants, anti-inflammatory agents, metabolic inhibitors, enzyme inhibitors, cytotoxic agents, and cytostatic agents. In another embodiment, the at least one additional therapeutic agent is selected from the group consisting of TNF antagonists, anti-TNF agents, IL-12 antagonists, IL-15 antagonists, IL-17 antagonists, IL-18 antagonists, IL-22 antagonists, T cell-depleting agents, B cell-depleting agents, cyclosporin, FK-506, CCI-779, etanercept, infliximab, rituximab, adalimumab, prednisolone, azathioprine, gold, sulphasalazine, chloroquine, hydroxychloroquine, minocycline, anakinra, abatacept, methotrexate, leflunomide, rapamycin, rapamycin analogs, Cox-2 inhibitors, cPLA2 inhibitors, NSAIDs, p38 inhibitors, antagonists of B7.1, B7.2, ICOSL, ICOS and/or CD28, and agonists of CTLA4.

In another embodiment, the invention provides a method of inhibiting the ability of NF-κB to activate NF-κB-responsive promoters in a cell population or a subject, comprising administering an IL-17F signaling antagonist to the cell population or the subject. In another embodiment, the invention provides a method for inhibiting an IL-17F bioactivity in a cell population or a subject, the method comprising administering an IL-17F signaling antagonist to the cell population or the subject. In another embodiment, the IL-17F bioactivity is selected from the group consisting of neutrophil differentiation, neutrophil recruitment and cytokine induction.

In another embodiment, the invention provides a pharmaceutical composition comprising an IL-17F signaling antagonist and a pharmaceutically acceptable carrier. In another embodiment, the invention provides a vaccine adjuvant comprising an IL-17F signaling antagonist and an antigen selected from the group consisting of an autoantigen, an allergen, an alloantigen, and fragments thereof. In another embodiment, the invention provides isolated antibodies capable of specifically binding to the amino acid sequences related to the present invention, including those set forth in SEQ ID NOs:6, 7, 9, 11, 13, and 15; in some embodiments, the antibody antagonizes IL-17F signaling.

In another embodiment, the invention provides an isolated antibody capable of specifically binding to IL-17F protein, and further embodiments wherein the IL-17F protein is derived from a human or a primate; wherein the IL-17F protein is multimeric; wherein the IL-17F protein is IL-17F homodimer or an IL-17F heterodimer; wherein the IL-17F heterodimer is IL-17A/IL-17F; and wherein the antibody inhibits IL-17F bioactivity.

In another embodiment, the invention provides the above-identified methods, wherein IL-17F signaling and/or IL-17F bioactivity is mediated by IL-17F homodimer, an IL-17F heterodimer, or both IL-17F homodimer and an IL-17F heterodimer, including wherein the IL-17F heterodimer is IL-17A/IL-17F.

In another embodiment, the invention provides the above-identified pharmaceutical composition and/or the above-identified vaccine adjuvant, wherein the IL-17F signaling antagonist antagonizes IL-17F homodimer, an IL-17F heterodimer, or both IL-17F homodimer and an IL-17F heterodimer.

In another embodiment, the invention provides a method of inhibiting at least one activity associated with IL-21 signaling comprising antagonizing IL-17F signaling. In another embodiment, the invention provides a method of inhibiting at least one activity associated with IL-23 signaling comprising antagonizing IL-17F signaling. In some further embodiments, the IL-17F signaling is mediated by IL-17F homodimer, an IL-17F heterodimer, or both IL-17F homodimer and an IL-17F heterodimer, including wherein the IL-17F heterodimer is IL-17A/IL-17F.

In another embodiment, the invention provides a method of purifying natural IL-17A protein comprising: activating T cells in media; and immunoprecipitating IL-17A protein from the media. In another embodiment, the invention provides a method of purifying natural IL-17F protein comprising: activating T cells in media; and immunoprecipitating IL-17F protein from the media. In some further embodiments, such methods are provided wherein the IL-17A protein is IL-17A homodimer, an IL-17A heterodimer, or both IL-17A homodimer and an IL-17A heterodimer, and/or wherein the IL-17F protein is IL-17F homodimer, an IL-17F heterodimer, or both IL-17F homodimer and an IL-17F heterodimer; and wherein the IL-17A or IL-17F heterodimer is IL-17A/IL-17F. In another embodiment, the media comprises IL-21 and/or IL-23.

In another embodiment, the invention provides an isolated IL-17F protein, wherein the IL-17F protein is IL-17F homodimer or an IL-17F heterodimer; wherein the IL-17F protein is isolated from a natural source; wherein the natural source is at least one T cell. In another embodiment, the invention provides an isolated IL-17A protein, wherein the IL-17A protein is IL-17A homodimer or an IL-17A heterodimer; wherein the IL-17A protein is isolated from a natural source; wherein the natural source is at least one T cell.

In another embodiment, the invention provides a method of inhibiting at least one activity associated with IL-17A signaling, comprising administering an IL-17F antagonist.

In another embodiment, the invention provides a method of isolating IL-17A/IL-17F heterodimers substantially free from IL-17A homodimers and IL-17F homodimers, comprising: (a) expressing an IL-17A fusion protein and an IL-17F fusion protein in host cells cultured in media, wherein the IL-17A fusion protein comprises an IL-17A protein or fragment thereof fused to a first affinity tag, and wherein the IL-17F fusion protein comprises an IL-17F protein or fragment thereof fused to a second affinity tag; (b) allowing the host cells to secrete the IL-17A fusion protein and IL-17F fusion protein into the media; (c) placing the media over a first affinity column under nonreducing conditions such that the IL-17A fusion protein binds to the first affinity column; (d) eluting the bound protein from the first affinity column under nonreducing conditions; (e) placing the eluent obtained from step (d) over a second affinity column under nonreducing conditions such that the IL-17F fusion protein binds to the second affinity column; and (f) eluting the bound protein from the second affinity column under nonreducing conditions, wherein the eluent obtained from step (f) contains both IL-17A fusion protein and IL-17F fusion protein in the form of IL-17A/IL-17F heterodimers. In other embodiments, variations of this method are provided. In another embodiment, the invention provides an IL-17A/IL-17F heterodimer isolated according to these various methods.

### BRIEF DESCRIPTION OF THE DRAWINGS

Shown in FIG. 1 is NF-κB-mediated reporter transactivation (Relative Luciferase Activity; y-axes) in (A) primary human chondrocytes or (B) primary porcine chondrocytes cultured in various concentrations (ng/ml) of IL-17A and/or IL-17F (x-axes).

The concentration (pg/ml; y-axis) of cytokines (IL-6, IL-8, MCP-1 or GRO-α; x-axis) from each of two patients (P1, P2; x-axis) in supernatant collected from human fibroblast-like synoviocytes cultured in media (control; □) or in the presence of 20 ng/ml IL-17F (IL-17F; ▪) is shown in FIG. 2.

FIG. 3 demonstrates the concentration (pg/ml; y-axis) of inflammatory cytokines (IL-6, JE (CCL2), KC; x-axis) in supernatants collected from cultures of primary murine lung fibroblasts cultured in media (0 ng/ml IL-17F; □), or with 1 ng/ml ( [symbol] ), 3.3. ng/ml ( [symbol] ), 10 ng/ml ( [symbol] ), or 30 ng/ml ( [symbol] ) IL-17F.

FIG. 4 demonstrates binding (OD 450 nm; y-axes) of increasing concentrations of human IL-17F (left panels) or human IL-17A (right panels) (x-axes) to (A) IL-17R-IgG (upper panels) or (B) IL-17RC-IgG (lower panels) as measured by ELISA. Also noted are EC₅₀ values for each receptor/cytokine interaction.

Shown in FIG. 5 is the concentration of GRO-α (pg/ml; y-axes) in supernatant collected from human fibroblasts cultured alone (Media; -) or with increasing concentrations (μg/ml; x-axes) of an IL-17R-IgG fusion protein (h17R.Fc; ), an IL-17RC-IgG fusion protein (h17RH2.Fc; ▪), a control IgG protein (hIgG1; ▴), an anti-IL-17R antibody (ahIL17R; □) or control antibody (goat IgG; Δ) in the presence of either (A) 0.5 ng/ml IL-17A (left panels) or (B) 20 ng/ml IL-17F (right panels).

FIG. 6 demonstrates the ability of anti-human IL-17F antibodies to inhibit the binding of IL-17F to IL-17R (OD 450 nm; y-axis) in the presence of increasing concentrations (μg/ml; x-axis) of one of the following six anti-IL-17F antibodies: anti-IL-17F-01 (□), anti-IL-17F-02 (-), anti-IL-17F-03 (▴), anti-IL-17F-05 (♦), anti-IL-17F-06 (), and anti-IL-17F-07 (Δ).

FIG. 7 demonstrates the ability of anti-human IL-17F antibodies to inhibit the binding of IL-17F to IL-17RC (OD 450 nm; y-axis) in the presence of increasing concentrations (μg/ml; x-axis) of each of the following six anti-IL-17F antibodies: anti-IL-17F-01 (□), anti-IL-17F-02 (-), anti-IL-17F-03 (▴), anti-IL-17F-05 (♦), anti-IL-17F-06 (), and anti-IL-17F-07 (Δ).

Shown in FIG. 8 is the concentration of GRO-α (pg/ml; y-axes) in supernatant collected from human fibroblasts cultured in 20 ng/ml IL-17F and increasing concentrations (μg/ml; x-axis) of (left panel) anti-IL-17F-01 (aIL-17F-01), anti-IL-17F-02 (aIL-17F-02), or anti-IL-17F-03 (aIL-17F-03) and (right panel) anti-IL-17F-05 (aIL-17F-05), anti-IL-17F-06 (aIL-17F-06), or anti-IL-17F-07 (aIL-17F-07), or control mIgG1 antibodies.

Shown in FIG. 9 is NF-κB-mediated reporter transactivation (Relative Luciferase Activity; y-axis) in porcine primary chondrocytes cultured in media only (none), in 100 ng/ml IL-17A (IL-17A(100 ng/ml)), in 100 ng/ml IL-17A in the presence of an IL-17R-IgG fusion protein (IL-17A+IL17R/Fc), in 100 ng/ml IL-17A in the presence of an anti-IL-17F antibody (IL17A+antiIL17F), in 100 ng/ml IL-17A in the presence of a control mouse IgG (IL-17A+mouseIgG), in 500 ng/ml IL-17F (IL-17F(500 ng/ml)), in 500 ng/ml IL-17F in the presence of an IL-17R-IgG fusion protein (IL-17F+IL17R/Fc), in 500 ng/ml IL-17F in the presence of an anti-IL-17F antibody (IL-17F+antiIL17F), or in 500 ng/ml IL-17F in the presence of a control mouse IgG (IL-17F+mouse IgG).

The concentration (pg/ml; y-axis) of cytokines (IL-6, IL-8, or GRO-α; x-axis) from each of two patients (P1, P2; x-axis) in supernatant collected from human fibroblast-like synoviocytes cultured in the presence of 20 ng/ml IL-17F (IL-17F; □), an isotype control antibody (Isotype Ab; [symbol] ), Anti-IL-17F-01 antibody ( [symbol] ), or Anti-IL-17F-07 antibody ( [symbol] ) is shown in FIG. 10.

FIG. 11 demonstrates the detection (OD 450 nm; y-axes) of IL-17A homodimers (IL-17A/A; x-axes), IL-17F homodimers (IL-17F/F; x-axes), or IL-17A/IL-17F heterodimers (IL-17A/F; x-axes) using ELISA formats specific for the detection of (A) IL-17A protein (including IL-17A homodimers and IL-17A heterodimers) (B) IL-17F protein (including IL-17F homodimers and IL-17F heterodimers), or (C) IL-17A/IL-17F heterodimers.

FIG. 12 demonstrates the concentration (Cytokine Produced (pg/ml); y-axes) of (A) IL-17A or (B) IL-17F in media isolated from T cells undergoing primary activation in the presence of bead-bound anti-CD3 antibody, increasing concentrations of anti-CD28 antibody (Anti-CD28 (ng/ml); x-axes), and in the absence (□) or presence of IL-21 ( [symbol] ) or IL-23 ( [symbol] ).

FIG. 13 demonstrates the concentration (Cytokine Produced (pg/ml); y-axis) of IL-17A ( [symbol] ) or IL-17F (□) in media isolated from T cells undergoing secondary activation under the following stimulating conditions (x-axis): IL-23 only (IL-23); IL-21 only (IL-21); bead-bound anti-CD3 antibody and anti-CD28 antibody (CD3/CD28); IL-23, bead-bound anti-CD3 antibody and anti-CD28 antibody (IL-23/CD3/CD28); IL-21, bead-bound anti-CD3 antibody and anti-CD28 antibody (IL-21/CD3/CD28); or media.

FIG. 14 demonstrates the detection (OD 450 nm; y-axes) of IL-17A homodimers, IL-17F homodimers, or IL-17A/IL-17F heterodimers in undiluted (neat) or diluted (1:10) media obtained from T cells subject to primary activation (CM1) or restimulation (CM2) (x-axes) using ELISA formats specific for the detection of (A) IL-17A protein (including IL-17A homodimers and IL-17A heterodimers), (B) IL-17F protein (including IL-17F homodimers and IL-17F heterodimers), or (C) IL-17A/IL-17F heterodimers.

Shown in FIG. 15 is a Western blot analysis performed with polyclonal rabbit anti-human IL-17F antibody to detect anti-human IL-17F-01 immunoprecipitates from 500 μl of conditioned media obtained from T cells undergoing secondary activation. Controls consist of IL-17F homodimer (second lane) prepared as described in Example 5.3, or IL-17A homodimers (fifth lane) purchased from R&D Systems (Minneapolis, Minn.). The molecular weight standard is shown in first lane. The positions of the IL-17A and IL-17F homodimers and IL-17F/IL-17A heterodimers are indicated by arrows.

FIG. 16 is the result of a Western blot analysis performed with biotin-conjugated goat anti-human IL-17A antibody to detect the anti-human IL-17A-02 immunoprecipitates from 500 μl of conditioned media obtained from T cells undergoing secondary activation. Control (lane 2) consists of IL-17F homodimer prepared as described in Example 5.3. The molecular weight standard is shown in lane 1. The positions of the IL-17A and IL-17F homodimers and IL-17F/IL-17A heterodimers are indicated by arrows.

FIG. 17A shows anti-IL-17F immunoprecipitates (lanes 2-7) or anti-IL-17A immunoprecipitates (lanes 8-10) immunoprobed with anti-IL-17F antibody. Immunoprecipitates were obtained from the conditioned media (CM) of COS cells overexpressing IL-17A (lanes 2 and 8), IL-17F (lanes 3 and 10), IL-17A and IL-17F (lanes 4 and 9), purified IL-17A homodimer (lane 5), or purified IL-17F homodimer (lanes 6 and 7). Controls (“A/A Purified,” lane 5, and “F/F purified,” lanes 6-7) consist of purified recombinant IL-17A and IL-17F homodimers as described in Example 5.4. The molecular weight standard is shown in lane 1. The positions of the IL-17A and IL-17F homodimers and IL-17F/IL-17A heterodimer are indicated by arrows.

FIG. 17B shows anti-IL-17A immunoprecipitates (lanes 2-4) or anti-IL-17F immunoprecipitates (lanes 5-7) immunoprobed with anti-IL-17A antibody. Immunoprecipitates were obtained from the conditioned media (CM) of COS cells overexpressing IL-17A (lanes 3 and 5), IL-17F (lanes 2 and 7), or IL-17A and IL-17F (lanes 4 and 6). The molecular weight standard is shown in lane 1. The positions of the IL-17A and IL-17F homodimers and IL-17F/IL-17A heterodimer are indicated by arrows.

FIG. 18 is a diagram showing a method of purifying recombinant IL-17F/IL-17A heterodimers substantially free from IL-17A and IL-17F homodimers. The method employs IL-17A and IL-17F with two different affinity tags, and uses two separate and sequential affinity columns to isolate IL-17F/IL-17A heterodimers.

FIG. 19A shows that recombinant purified IL-17F/IL-17A heterodimers (X), similar to IL-17A (♦) and IL-17F (□) homodimers, stimulate GRO-α levels (pg/ml) in the media of BJ cell cultures. FIG. 19B shows that cotreatment of BJ cultures with anti-IL-17A antibody (▪), or anti-IL-17A in combination with anti-IL-17F antibodies (Δ), but not IL-17F antibodies alone (□), abrogates IL-17F/IL-17A heterodimer stimulation of GRO-α levels. Controls consisted of cultures provided with media lacking both IL-17F and IL-17A antibodies (X).

FIG. 20 is a table summarizing MALDI-TOF mass spectrometry data for tryptic peptide masses prepared by digestion of IL-17F homodimers, IL-17A homodimers, and IL-17F/IL-17A heterodimers. The first column of the table shows the origin of the peptide fragment analyzed, the second column (Structure) shows the peptide fragment sequence, the third column (MW Cal) shows the calculated molecular weight of the fragment, the fourth column shows the calculated mass-to-charge ratio (m/z value) of the fragment (Calculated), and the fifth column shows the actual mass-to-charge ratio (m/z value) (Observed) as determined by mass spectrometry.

FIG. 21 shows that anti-human IL-17F antibodies can partially inhibit the biological activity of primate IL-17F. FIGS. 21A and 21B show that BJ cells stimulated with human or primate (macaque) IL-17F display increased levels of GRO-α in response to increasing levels of IL-17F. FIG. 21A shows that anti-IL-17F-01 (□) and anti-IL-17F-07 (X) antibodies decrease the ability of human IL-17F (♦) to stimulate GRO-α levels. Similarly, FIG. 21B shows that anti-IL-17F-01 (□) and anti-IL-17F-07 (X) antibodies decrease the ability of primate IL-17F (♦) to stimulate GRO-α levels, albeit to a lesser extent than the antibodies reduce human IL-17F biological activity.

FIG. 22 shows that IL-17F treatment increases the expression of ADAMTS-4 (Aggrecanase 1) in chondrocytes obtained from human donors, and that treatment with anti-IL-17F antibodies abrogates this stimulation. Cultured chondrocytes were treated with 250 ng/ml IL-17F, 250 ng/ml IL-17F and 25 μg/ml anti-IL-17F, 25 μg/ml anti-IL-17F, 250 ng/ml IL-17F and 25 μg/ml control IgG1, or 25 μg/ml control IgG1 (x-axis), and transcript levels of Aggrecanase 1 measured by real-time PCR (expressed as TAQMAN® units; y-axis). GAPDH expression levels were used as normalizer.

FIG. 23 shows that treatment of BJ cells with siRNA directed to transcripts of IL-17R and IL-17RC reduces the ability of IL-17F and IL-17A to increase GRO-α levels FIG. 23A: Taqman=% reduction in IL-17R transcript levels in cells treated with siRNA to IL-17R; IL-17F=% reduction in the ability of IL-17F to stimulate GRO-α levels in cells treated with siRNA to IL-17R; IL-17A=% reduction in the ability of IL-17A to stimulate GRO-α levels in cells treated with siRNA to IL-17R. FIG. 23B shows that treatment of BJ cells with siRNA directed to transcripts of IL-17RC reduces the ability of IL-17F and IL-17A to increase GRO-α levels. Taqman=% reduction in IL-17RC transcript levels in cells treated with siRNA to IL-17RC; IL-17F=% reduction in the ability of IL-17F to stimulate GRO-α levels in cells treated with siRNA to IL-17RC; IL-17A=% reduction in the ability of IL-17A to stimulate GRO-α levels in cells treated with siRNA to IL-17RC. FIG. 23C discloses several siRNA molecules of the present invention (SEQ ID NOs:17-32) that target mRNA polynucleotides related to the present invention (i.e., IL-17R and IL-17RC).

FIG. 24 shows the average fold-change (lesional/nonlesional (nonaffected) tissues) of IL-17F and IL-17A transcript expression in 48 pairs of tissue biopsy samples from patients suffering from psoriasis. Both IL-17A and IL-17F transcript levels are increased in psoriatic lesional tissues with respect to nonaffected tissue. P-values from paired t-tests are as follows: IL-17A p=2.8×10⁻³, IL-17F p=1.1×10⁻⁹.

FIG. 25 shows the average fold-change (involved/noninvolved tissues) of IL-17F and IL-17A transcript expression in paired tissue biopsy samples from patients suffering from ulcerative colitis (UC) (□) (12 pairs) or Crohn's disease (CD) (▪) (16 pairs). Both IL-17A and IL-17F transcript levels are increased in affected tissues relative to noninvolved tissues in both sets of IBD samples. P-values from paired t-tests are as follows: IL-17A (UC), p=0.309; IL-17A (CD), p=0.069; IL-17F (UC), p=0.406; IL-17F (CD), p=0.206.

FIG. 26 shows intracellular cytokine staining for IL-17F. Staining for IL-17F was performed on (lymph node) LN cells from C57BL/6 mice immunized with 100 μg ovalbumin emulsified in complete Freund's adjuvant. Cells were surface-stained for CD4, fixed, permeabilized and stained with an anti-IgG1 isotype control or with rat anti-murine IL-17F (clone 15-1). Numbers denote percent of positive cells.

### DETAILED DESCRIPTION OF THE INVENTION

Interleukin-17F (IL-17F) is a cytokine that belongs to the IL-17 family of proteins and induces expression of inflammatory cytokines and chemokines, e.g., IL-6, IL-8, GM-CSF, G-CSF, GRO-α, MCP-1, IL-1β, TNF-α, TGF-β, etc. Expression of IL-17F is correlated with neutrophilia and various autoimmune diseases (Bettelli and Kuchroo, supra). For example, IL-17F is associated with increased proteoglycan breakdown and decreased proteoglycan synthesis by articular cartilage (Hymowitz, supra), central nervous system autoimmunity (Langrish, supra), allergic and asthmatic responses (Kawaguchi, supra) and inflammatory bowel diseases (Gurney, supra). Thus, IL-17F signaling is believed to be involved with disorders including, but not limited to, inflammatory disorders, such as autoimmune diseases (e.g., arthritis (including rheumatoid arthritis), psoriasis, systemic lupus erythematosus (SLE), multiple sclerosis), respiratory diseases (e.g., COPD, cystic fibrosis, asthma, allergy), transplant rejection (including solid organ transplant rejection), and inflammatory bowel diseases (e.g., ulcerative colitis, Crohn's disease).

As part of the invention, the inventors have confirmed involvement of IL-17F in inflammatory disorders by demonstrating the following responses to administration of IL-17F: e.g., neutrophil influx into the peritoneum (Example 1.1), activation of a primary transcription factor of inflammatory cytokines correlated with an increased secretion of inflammatory cytokines by primary chondrocytes (Example 1.2), increased secretion of inflammatory cytokines by lung fibroblasts (Example 1.3), and increased levels of Aggrecanase in primary human chondrocytes (Example 7). The inventors have also determined that both IL-17F and IL-17A may be involved in autoimmune arthritis (Example 7), psoriasis (Example 9) and inflammatory bowel disease (IBD) (Example 9). The inventors have also identified IL-17R and IL-17RC as receptors for IL-17F (Example 2), thus providing novel targets for inhibition of the IL-17F signaling pathway. The inventors have also generated and characterized anti-IL-17F antibodies in terms of each antibody's binding specificity, affinity, and ability to inhibit IL-17F signaling, i.e., IL-17F bioactivity (Examples 3 and 5). In one embodiment, antibodies help to characterize IL-17F epitopes that may be required for IL-17R and/or IL-17RC recognition; i.e., five of six murine anti-human IL-17F antibodies are able to interfere with binding of IL-17F to IL-17R, and two of the five are also able to interfere with binding of IL-17F to IL-17RC. The inventors have also demonstrated the ability of some of these antibodies to inhibit (i.e., decrease, limit, block, or otherwise reduce) IL-17F bioactivities, e.g., IL-17F-mediated activation of a primary transcription factor for inflammatory cytokines, and subsequently, IL-17F-mediated cytokine secretion by primary fibroblast-like synoviocytes (Example 4). Also disclosed herein are inhibitory polynucleotides that decrease IL-17A and IL-17F signaling through the IL-17R and IL-17RC (Example 8). The inventors have also demonstrated a direct relationship between IL-21 and IL-17F, i.e., the ability of IL-21 to enhance the production of both IL-17A and IL-17F by activated T cells. Thus, it is reasoned that inhibition of IL-17F signaling may also inhibit at least one effect associated with IL-21 binding to and activation of IL-21R, e.g., methods of inhibiting IL-17F signaling may be used in methods of treating IL-17F-associated disorders and/or disorders associated with IL-21 binding to and activating IL-21R. The inventors also isolated for the first time IL-17A and IL-17F from the cytokines' natural source. The inventors have also demonstrated and purified a novel IL-17A/IL-17F heterodimer (e.g., in T cells, and HEK-293 and COS cells, respectively), and have shown that the heterodimer transduces IL-17F signaling, e.g., by inducing expression of GRO-α levels (Example 5). Thus the inventors have provided the heterodimer as a novel target for inhibition of the IL-17F-signaling pathway and/or in the treatment of inflammatory disorders and/or disorders associated with IL-21 binding to and activating IL-21R.

As such, the present invention provides IL-17F signaling antagonists, (e.g., IL-17F, IL-17R, and/or IL-17RC inhibitory polynucleotides; soluble IL-17R and/or IL-17RC polypeptides (including fragments (e.g., IL-17F binding fragments) and/or fusion proteins thereof); inhibitory anti-IL-17F, anti-IL-17R, or IL-17RC antibodies; and/or antagonistic small molecules), which may be used to suppress IL-17F-mediated (including IL-17F homodimer- and IL-17A/IL-17F heterodimer-mediated) inflammatory responses in vivo, and consequently, which may be used in the diagnosis, prognosis, monitoring and/or treatment of disorders related to increased IL-17F signaling, i.e., IL-17F-associated disorders and/or disorders associated with IL-21 binding to and activating IL-21R. The identification and isolation of the novel IL-17A/IL-17F heterodimer indicates that disorders related to IL-17F signaling may be mediated by IL-17F homodimers and/or IL-17F heterodimers. Thus the term “IL-17F” as used herein, where appropriate, refers to IL-17F homodimers or IL-17A/IL-17F heterodimers, e.g., the IL-17F signaling pathway encompasses a signaling pathway that may comprise either or both IL-17F homodimers and IL-17A/IL-17F heterodimers.

Accordingly, the present application provides IL-17F signaling-related polynucleotides and polypeptides, including IL-17R and IL-17RC polynucleotides and polypeptides. The present invention also provides antibodies, i.e., intact antibodies and antigen-binding fragments thereof, that bind to IL-17F, in particular, human IL-17F, including, but not limited to, IL-17F homodimers and IL-17A/IL-17F heterodimers. In one embodiment, an anti-IL-17F antibody inhibits or antagonizes at least one IL-17F-associated (e.g., IL-17F homodimer and/or IL-17A/IL-17F heterodimer) activity. For example, the anti-IL-17F antibody can bind to IL-17F and interfere with, e.g., block, an interaction between IL-17F and an IL-17F receptor complex, e.g., complexes comprising IL-17R and/or IL-17RC. Thus, the antibodies of the invention may be used detect, and optionally inhibit (e.g., decrease, limit, block or otherwise reduce), an IL-17F bioactivity, e.g., binding between IL-17F and an IL-17F receptor complex, or subunit thereof. Thus, the anti-IL-17F antibodies of the invention may be used to diagnose, prognose, monitor and/or treat or prevent disorders related to IL-17F signaling and/or disorders associated with IL-21 binding to and activating IL-21R.

### Polynucleotides and Polypeptides of IL-17F, IL-17R, and IL-17RC

The present invention provides further characterization of the IL-17F signaling pathway, i.e., determination of IL-17R and/or IL-17RC as an IL-17F receptor, elucidation of the effects of interfering with IL-17F binding to IL-17R and/or IL-17RC using inhibitory molecules, e.g., antibodies, receptor fusion proteins and siRNA, and the purification of IL-17A/IL-17F heterodimers. As such, the present invention relates to IL-17F, IL-17R, and IL-17RC polynucleotides and polypeptides, including inhibitory IL-17F, IL-17R and IL-17RC polynucleotides and polypeptides.

IL-17F nucleotide and amino acid sequences are known in the art and are provided. The nucleotide sequence of human IL-17F is set forth in SEQ ID NO:1. The amino acid sequence of full-length IL-17F protein coded by that nucleotide sequence is set forth in SEQ ID NO:2. The amino acid sequence of mature IL-17F corresponds to a protein beginning at about amino acid 31 of SEQ ID NO:2 (see, e.g., U.S. patent application Ser. No. 10/102,080, incorporated herein in its entirety by reference).

IL-17A nucleotide and amino acid sequences are known in the art and are provided. The nucleotide sequence of human IL-17A is set forth in SEQ ID NO:3, which includes a poly(A) tail. The amino acid sequence of full-length IL-17A protein corresponding to that nucleotide sequence is set forth in SEQ ID NO:4.

IL-17R nucleotide and amino acid sequences are known in the art and are provided. The nucleotide sequence of human IL-17R is set forth as SEQ ID NO:5, which includes a poly(A) tail. The amino acid sequence of full-length IL-17R protein corresponding to that nucleotide sequence is set forth in SEQ ID NO:6.

IL-17RC nucleotide and amino acid sequences are known in the art and are provided. The nucleotide sequences of several human IL-17RC polynucleotides, which include poly(A) tails, are set forth as SEQ ID NOs:7, 9, 11, 13, and 15. The amino acid sequences of several full-length human IL-17RC proteins corresponding to those nucleotide sequences are set forth in SEQ ID NOs:8, 10, 12, 14, and 16.

The nucleic acids related to the present invention may comprise DNA or RNA and may be wholly or partially synthetic. Reference to a nucleotide sequence as set forth herein encompasses a DNA molecule with the specified sequence (or a complement thereof), and encompasses an RNA molecule with the specified sequence in which U is substituted for T, unless context requires otherwise.

The isolated polynucleotides related to the present invention may be used as hybridization probes and primers to identify and isolate nucleic acids having sequences identical to or similar to those encoding the disclosed polynucleotides. Hybridization methods for identifying and isolating nucleic acids include polymerase chain reaction (PCR), Southern hybridization, in situ hybridization and Northern hybridization, and are well known to those skilled in the art.

Hybridization reactions may be performed under conditions of different stringency. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1
Stringency Conditions

| Stringency | Polynucleotide | Hybrid | Hybridization Temperature | Wash Temperature |
|---|---|---|---|---|
| Condition | Hybrid | Length (bp)¹ | and Buffer² | and Buffer² |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{F}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete.
T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ(°C. = 81.5 + 16.6(log₁₀Na⁺) + 0.41(% G + C) − (600/N), where N is the number of bases in the hybrid, and Na⁺ is the concentration of sodium ions in the hybridization buffer (Na⁺ for 1xSSC = 0.165M).
Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, NY, chapters 9 and 11, and Current Protocols in Molecular Biology, 1995, P. M. Ausubel et al., eds., John Wiley & Sons, Inc., sections 2.10 and 6.3-6.4, incorporated herein by reference.

The isolated polynucleotides related to the present invention may be used as hybridization probes and primers to identify and isolate DNA having sequences encoding allelic variants of the disclosed polynucleotides. Allelic variants are naturally occurring alternative forms of the disclosed polynucleotides that encode polypeptides that are identical to or have significant similarity to the polypeptides encoded by the disclosed polynucleotides. Preferably, allelic variants have at least 90% sequence identity (more preferably, at least 95% identity; most preferably, at least 99% identity) with the disclosed polynucleotides. Alternatively, significant similarity exists when the nucleic acid segments will hybridize under selective hybridization conditions (e.g., highly stringent hybridization conditions) to the disclosed polynucleotides.

The isolated polynucleotides related to the present invention may also be used as hybridization probes and primers to identify and isolate DNAs having sequences encoding polypeptides homologous to the disclosed polynucleotides. These homologs are polynucleotides and polypeptides isolated from a different species than that of the disclosed polypeptides and polynucleotides, or within the same species, but with significant sequence similarity to the disclosed polynucleotides and polypeptides. Preferably, polynucleotide homologs have at least 50% sequence identity (more preferably, at least 75% identity; most preferably, at least 90% identity) with the disclosed polynucleotides, whereas polypeptide homologs have at least 30% sequence identity (more preferably, at least 45% identity; most preferably, at least 60% identity) with the disclosed polypeptides. Preferably, homologs of the disclosed polynucleotides and polypeptides are those isolated from mammalian species.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and nonhomologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent sequence identity between two sequences may be accomplished using a mathematical algorithm. In a preferred embodiment, the percent identity between two amino acid sequences is determined using the Needleman and Wunsch ((1970) J. Mol. Biol. 48:444-53) algorithm, which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine whether a molecule is within a sequence identity or homology limitation of the invention) is a Blossum 62 scoring matrix with a gap penalty of 12, a gap extend penalty of 4, and a frameshift gap penalty of 5. The percent identity between two amino acid or nucleotide sequences can also be determined using the algorithm of Meyers and Miller ((1989) CABIOS 4:11-17), which has been incorporated into the ALIGN program (version 2.0), using a PAM120 weight residue table, a gap length penalty of 12 and a gap penalty of 4.

The isolated polynucleotides related to the present invention may also be used as hybridization probes and primers to identify cells and tissues that express the polypeptides related to the present invention and the conditions under which they are expressed.

Additionally, the function of the polypeptides related to the present invention may be directly examined by using the polynucleotides encoding the polypeptides to alter (i.e., enhance, reduce, or modify) the expression of the genes corresponding to the polynucleotides related to the present invention in a cell or organism. These “corresponding genes” are the genomic DNA sequences related to the present invention that are transcribed to produce the mRNAs from which the polynucleotides related to the present invention are derived.

Altered expression of the genes related to the present invention may be achieved in a cell or organism through the use of various inhibitory polynucleotides, such as antisense polynucleotides, siRNAs, and ribozymes that bind and/or cleave the mRNA transcribed from the genes related to the invention (see, e.g., Galderisi et al. (1999) J. Cell Physiol. 181:251-57; Sioud (2001) Curr. Mol. Med. 1:575-88). Inhibitory polynucleotides to, e.g., IL-17F, IL-17R, and/or IL-17RC, may be useful as IL-17F signaling antagonists and, as such, may also be useful in preventing or treating disorders related to IL-17F signaling. Inhibitory polynucleotides may also consist of aptamers, i.e., polynucleotides that bind to and regulate protein activity, e.g., the activity of IL-17F, IL-17A, IL-17R, and/or IL-17RC. Aptamers are described throughout the literature, see, e.g., Nimjee et al. (2005) Annu. Rev. Med. 56:555-83 and Patel (1997) Curr. Opin. Chem. Biol. 1:32-46.

The antisense polynucleotides or ribozymes related to the invention may be complementary to an entire coding strand of a gene related to the invention, or to only a portion thereof. Alternatively, antisense polynucleotides or ribozymes can be complementary to a noncoding region of the coding strand of a gene related to the invention. The antisense polynucleotides or ribozymes can be constructed using chemical synthesis and enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al., supra; Heasman (2002) Dev. Biol. 243:209-14; Micklefield (2001) Curr. Med. Chem. 8:1157-79). Alternatively, these molecules can be produced biologically using an expression vector into which a polynucleotide related to the present invention has been subcloned in an antisense (i.e., reverse) orientation.

The inhibitory polynucleotides of the present invention also include triplex-forming oligonucleotides (TFOs) that bind in the major groove of duplex DNA with high specificity and affinity (Knauert and Glazer (2001) Hum. Mol. Genet. 10:2243-51). Expression of the genes related to the present invention can be inhibited by targeting TFOs complementary to the regulatory regions of the genes (i.e., the promoter and/or enhancer sequences) to form triple helical structures that prevent transcription of the genes.

In one embodiment of the invention, the inhibitory polynucleotides of the present invention are short interfering RNA (siRNA) molecules. These siRNA molecules are short (preferably 19-25 nucleotides; most preferably 19 or 21 nucleotides), double-stranded RNA molecules that cause sequence-specific degradation of target mRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNA molecules targeted to Fas mRNA (Song et al. (2003) Nature Med. 9:347-51). In addition, intrathecally delivered siRNA has recently been reported to block pain responses in two models (agonist-induced pain model and neuropathic pain model) in the rat (Dorn et al. (2004) Nucleic Acids Res. 32(5):e49).

The siRNA molecules of the present invention may be generated by annealing two complementary single-stranded RNA molecules together (one of which matches a portion of the target mRNA) (Fire et al., U.S. Pat. No. 6,506,559) or through the use of a single hairpin RNA molecule that folds back on itself to produce the requisite double-stranded portion (Yu et al. (2002) Proc. Natl. Acad. Sci. USA 99:6047-52). The siRNA molecules may be chemically synthesized (Elbashir et al. (2001) Nature 411:494-98) or produced by in vitro transcription using single-stranded DNA templates (Yu et al., supra). Alternatively, the siRNA molecules can be produced biologically, either transiently (Yu et al., supra; Sui et al. (2002) Proc. Natl. Acad. Sci. USA 99:5515-20) or stably (Paddison et al. (2002) Proc. Natl. Acad. Sci. USA 99:1443-48), using an expression vector(s) containing the sense and antisense siRNA sequences. Recently, reduction of levels of target mRNA in primary human cells, in an efficient and sequence-specific manner, was demonstrated using adenoviral vectors that express hairpin RNAs, which are further processed into siRNAs (Arts et al. (2003) Genome Res. 13:2325-32).

The siRNA molecules targeted to the polynucleotides related to the present invention can be designed based on criteria well known in the art (e.g., Elbashir et al. (2001) EMBO J. 20:6877-88). For example, the target segment of the target mRNA preferably should begin with AA (most preferred), TA, GA, or CA; the GC ratio of the siRNA molecule preferably should be 45-55%; the siRNA molecule preferably should not contain three of the same nucleotides in a row; the siRNA molecule preferably should not contain seven mixed G/Cs in a row; and the target segment preferably should be in the ORF region of the target mRNA and preferably should be at least 75 by after the initiation ATG and at least 75 by before the stop codon. Based on these criteria, or on other known criteria (e.g., Reynolds et al. (2004) Nature Biotechnol. 22:326-30), siRNA molecules of the present invention that target the mRNA polynucleotides related to the present invention may be designed by one of ordinary skill in the art. Preferred examples of siRNAs for use in the disclosed methods are set forth in SEQ ID NOs:17-32 and correspond to siRNAs useful to target IL-17R (SEQ ID NOs:17-24) and IL-17RC (SEQ ID NOs:25-32).

Altered expression of the genes related to the present invention in an organism may also be achieved through the creation of nonhuman transgenic animals into whose genomes polynucleotides related to the present invention have been introduced. Such transgenic animals include animals that have multiple copies of a gene (i.e., the transgene) of the present invention. A tissue-specific regulatory sequence(s) may be operably linked to the transgene to direct expression of a polypeptide related to the present invention to particular cells or a particular developmental stage. Methods for generating transgenic animals via embryo manipulation and microinjection, particularly animals such as mice, have become conventional and are well known in the art (e.g., Bockamp et al., Physiol. Genomics 11:115-32 (2002)).

Altered expression of the genes related to the present invention in an organism may also be achieved through the creation of animals whose endogenous genes corresponding to the polynucleotides related to the present invention have been disrupted through insertion of extraneous polynucleotide sequences (i.e., a knockout animal). The coding region of the endogenous gene may be disrupted, thereby generating a nonfunctional protein. Alternatively, the upstream regulatory region of the endogenous gene may be disrupted or replaced with different regulatory elements, resulting in the altered expression of the still-functional protein. Methods for generating knockout animals include homologous recombination and are well known in the art (e.g., Wolfer et al., Trends Neurosci. 25:336-40 (2002)).

The isolated polynucleotides of the present invention also may be operably linked to an expression control sequence and/or ligated into an expression vector for recombinant production of the polypeptides (including active fragments and/or fusion polypeptides thereof) related to the present invention. General methods of expressing recombinant proteins are well known in the art.

An expression vector, as used herein, is intended to refer to a nucleic acid molecule capable of transporting another nucleic acid to which it has been linked. One type of vector is a plasmid, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., nonepisomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as recombinant expression vectors (or simply, expression vectors). In general, expression vectors of utility in recombinant DNA techniques are often in the form of plasmids. In the present specification, plasmid and vector may be used interchangeably as the plasmid is the most commonly used form of vector. However, the invention is intended to include other forms of expression vectors, such as viral vectors (e.g., replication defective retroviruses, adenoviruses and adeno-associated viruses) that serve equivalent functions.

In one embodiment, the polynucleotides related to the present invention are used to create recombinant IL-17F agonists, e.g., those that can be identified based on the presences of at least one “IL-17F receptor-binding motif.” As used herein, the term “IL-17F receptor-binding motif” includes amino acid sequences or residues that are important for binding of IL-17F to its requisite receptor. An example of an IL-17F agonist includes IL-17F homodimer, IL-17A/IL-17F heterodimer, fragments thereof, e.g., IL-17R or IL-17RC binding fragments, and/or small molecules (as described below). Such agonists may be useful in regulation of hematopoiesis, and consequently, in the treatment of myeloid or lymphoid cell deficiencies. In another embodiment, the polynucleotides related to the present invention are used to create IL-17F signaling antagonists (e.g., IL-17F, IL-17R, and/or IL-17RC inhibitory polynucleotides; soluble IL-17R and/or IL-17RC polypeptides (including fragments (e.g., IL-17F binding fragments) and/or fusion proteins thereof); inhibitory anti-IL-17F, anti-IL-17R, or IL-17RC antibodies, which may inhibit the bioactivity of IL-17F homodimers and/or IL-17A/IL-17F heterodimers; and/or antagonistic small molecules, etc.).

Methods of creating fusion polypeptides, i.e., a first polypeptide moiety linked with a second polypeptide moiety, are well known in the art. For example, an IL-17F polypeptide or an IL-17F receptor polypeptide (e.g., IL-17R and/or IL-17RC, including fragments thereof) may be fused to a second polypeptide moiety, e.g., an immunoglobulin or a fragment thereof (e.g., an Fc binding fragment thereof). In some embodiments, the first polypeptide moiety includes, e.g., full-length IL-17RC polypeptide. Alternatively, the first polypeptide may comprise less than the full-length IL-17RC polypeptide. Additionally, soluble forms of, e.g., IL-17RC may be fused through “linker” sequences to the Fc portion of an immunoglobulin. Other fusions proteins, such as those with glutathione-S-transferase (GST), Lex-A, thioredoxin (TRx) or maltose-binding protein (MBP), may also be used.

The second polypeptide moiety is preferably soluble. In some embodiments, the second polypeptide moiety enhances the half-life, (e.g., the serum half-life) of the linked polypeptide. In some embodiments, the second polypeptide moiety includes a sequence that facilitates association of the fusion polypeptide with a second IL-17F or IL-17R polypeptide. In preferred embodiments, the second polypeptide includes at least a region of an immunoglobulin polypeptide. Immunoglobulin fusion polypeptide are known in the art and are described in, e.g., U.S. Pat. Nos. 5,516,964; 5,225,538; 5,428,130; 5,514,582; 5,714,147; and 5,455,165, all of which are hereby incorporated by reference. The fusion proteins may additionally include a linker sequence joining the first polypeptide moiety, e.g., IL-17F or IL-17R, including fragments thereof, to the second moiety. Use of such linker sequences are well known in the art. For example, the fusion protein can include a peptide linker, e.g., a peptide linker of about 2 to 20, more preferably less than 10, amino acids in length. In one embodiment, the peptide linker may be 2 amino acids in length.

In another embodiment, the recombinant protein includes a heterologous signal sequence (i.e., a polypeptide sequence that is not present in a polypeptide encoded by an IL-17F, IL-17R or IL-17RC nucleic acid) at its N-terminus. For example, a signal sequence from another protein may be fused with an IL-17R and/or IL-17RC polypeptide, including fragments and/or fusion proteins thereof. In certain host cells (e.g., mammalian host cells), expression and/or secretion of recombinant proteins can be increased through use of a heterologous signal sequence. A signal peptide that may be included in the fusion protein is the melittin signal peptide MKFLVNVALVFMVVYISYIYA (SEQ ID NO:33).

A fusion protein of the invention may be produced by standard recombinant DNA techniques. For example, DNA fragments coding for the different polypeptide sequences are ligated together in-frame in accordance with conventional techniques by employing, e.g., blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments may be carried out using anchor primers that give rise to complementary overhangs between two consecutive gene fragments that can subsequently be annealed and reamplified to generate a chimeric gene sequence (see, for example, Ausubel et al. (Eds.) Current Protocols in Molecular Biology, John Wiley & Sons, 1992). Moreover, many expression vectors are commercially available that encode a fusion moiety (e.g., an Fc region of an immunoglobulin heavy chain). An IL-17F-, IL-17R- and/or IL-17RC-encoding nucleic acid may be cloned into such an expression vector such that the fusion moiety is linked in-frame to the immunoglobulin protein. In some embodiments, IL-17F, IL-17R and/or IL-17RC fusion polypeptides exist as oligomers, such as dimers or trimers.

The recombinant expression vectors of the invention may carry additional sequences, such as sequences that regulate replication of the vector in host cells (e.g., origins of replication) and selectable marker genes. The selectable marker gene facilitates selection of host cells into which the vector has been introduced. For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

Suitable vectors can be chosen or constructed, containing appropriate regulatory sequences, including promoter sequences, terminator sequences, polyadenylation sequences, enhancer sequences, marker genes and other sequences, e.g., sequences that regulate replication of the vector in the host cells (e.g., origins of replication) as appropriate. Vectors may be plasmids or viral, e.g., phage, or phagemid, as appropriate. For further details see, for example, Molecular Cloning: a Laboratory Manual: 2nd ed., Sambrook et al., Cold Spring Harbor Laboratory Press, 1989. Many known techniques and protocols for manipulation of nucleic acid, for example, in preparation of nucleic acid constructs, mutagenesis, sequencing, introduction of DNA into cells and gene expression, and analysis of proteins, are described in detail in Current Protocols in Molecular Biology, 2nd ed., Ausubel et al. eds., John Wiley & Sons, 1992.

Thus, a further aspect of the present invention provides a host cell comprising a nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection, and transduction using retrovirus or other viruses, e.g., vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene.

A number of cell lines may act as suitable host cells for recombinant expression of the polypeptides related to the present invention. Mammalian host cell lines include, for example, COS cells, CHO cells, 293 cells, A431 cells, 3T3 cells, CV-1 cells, HeLa cells, L cells, BHK21 cells, HL-60 cells, U937 cells, HaK cells, Jurkat cells, as well as cell strains derived from in vitro culture of primary tissue and primary explants.

Alternatively, it may be possible to recombinantly produce the polypeptides related to the present invention in lower eukaryotes, such as yeast, or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the polypeptides related to the present invention are made in yeast or bacteria, it may be necessary to modify them by, for example, phosphorylation or glycosylation of appropriate sites, in order to obtain functionality. Such covalent attachments may be accomplished using well-known chemical or enzymatic methods.

Expression in bacteria may result in formation of inclusion bodies incorporating the recombinant protein. Thus, refolding of the recombinant protein may be required in order to produce active or more active material. Several methods for obtaining correctly folded heterologous proteins from bacterial inclusion bodies are known in the art. These methods generally involve solubilizing the protein from the inclusion bodies, then denaturing the protein completely using a chaotropic agent. When cysteine residues are present in the primary amino acid sequence of the protein, it is often necessary to accomplish the refolding in an environment that allows correct formation of disulfide bonds (a redox system). General methods of refolding are disclosed in Kohno (1990) Meth. Enzymol. 185:187-95. EP 0433225, and U.S. Pat. No. 5,399,677 describe other appropriate methods.

The polypeptides related to the present invention may also be recombinantly produced by operably linking the isolated polynucleotides of the present invention to suitable control sequences in one or more insect expression vectors, such as baculovirus vectors, and employing an insect cell expression system. Materials and methods for baculovirus/Sf9 expression systems are commercially available in kit form (e.g., MAXBAC® kit, Invitrogen, Carlsbad, Calif.).

Following recombinant expression in the appropriate host cells, the recombinant polypeptides of the present invention may then be purified from culture medium or cell extracts using known purification processes, such as immunoprecipitation, gel filtration and ion exchange chromatography. For example, soluble forms of IL-17F signaling antagonists, e.g., IL-17R protein and/or IL-17RC proteins (including fragments, and/or fusion proteins thereof); or IL-17F agonists, e.g., soluble IL-17F (in homodimer or IL-17A/IL-17F heterodimer formation), may be purified from conditioned media. Membrane-bound forms of, e.g., an IL-17F signaling antagonist, may be purified by preparing a total membrane fraction from the expressing cell and extracting the membranes with a nonionic detergent such as Triton X-100. A polypeptide related to the present invention may be concentrated using a commercially available protein concentration filter, for example, an AMICON® or Millipore PELLICON® ultrafiltration unit (Millipore, Billerica, Mass.). Following the concentration step, the concentrate can be applied to a purification matrix such as a gel filtration medium. Alternatively, an anion exchange resin can be employed, for example, a matrix or substrate having pendant diethylaminoethyl (DEAE) or polyetheyleneimine (PEI) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred (e.g., S-SEPHAROSE® columns, Sigma-Aldrich, St. Louis, Mo.). The purification of recombinant proteins from culture supernatant may also include one or more column steps over such affinity resins as concanavalin A-agarose, heparin-TOYOPEARL® (Togo Soda Manufacturing Co., Ltd., Japan) or Cibacrom blue 3GA SEPHAROSE® (Tosoh Biosciences, San Francisco, Calif.); or by hydrophobic interaction chromatography using such resins as phenyl ether, butyl ether, or propyl ether; or by immunoaffinity chromatography. Finally, one or more reverse-phase high performance liquid chromatography (RP-HPLC) steps employing hydrophobic RP-HPLC media, e.g., silica gel having pendant methyl or other aliphatic groups, can be employed to further purify the recombinant protein. Affinity columns including antibodies (e.g., those described using the methods herein) to the recombinant protein may also be used in purification in accordance with known methods. Some or all of the foregoing purification steps, in various combinations or with other known methods, may also be employed to provide a substantially purified isolated recombinant protein. Preferably, the isolated recombinant protein is purified so that it is substantially free of other mammalian proteins. Additionally, these purification processes may also be used to purify the polypeptides of the present invention from other sources, including natural sources. For example, polypeptides related to the invention, e.g., IL-17F agonists (e.g., soluble IL-17F) or IL-17F signaling antagonists (e.g., soluble IL-17R and/or soluble IL-17RC proteins, including fragments and/or fusion proteins thereof), which are expressed as a product of transgenic animals, e.g., as a component of the milk of transgenic cows, goats, pigs, or sheep, may be purified as described above.

Alternatively, the polypeptides may also be recombinantly expressed in a form that facilitates purification. For example, the polypeptides may be expressed as fusions with proteins such as maltose-binding protein (MBP), glutathione-S-transferase (GST), or thioredoxin (TRX). Kits for expression and purification of such fusion proteins are commercially available from New England BioLabs (Beverly, Mass.), Pharmacia (Piscataway, N.J.), and Invitrogen, respectively. Recombinant proteins can also be tagged with a small epitope and subsequently identified or purified using a specific antibody to the epitope. A preferred epitope is the FLAG epitope, which is commercially available from Eastman Kodak (New Haven, Conn.).

Alternatively, recombinant IL-17F and IL-17A fusion proteins may be tagged with different epitopes to allow purification of IL-17A/IL-17F heterodimers. The existence of different tags on IL-17F and IL-17A allows isolation of IL-17A/IL-17F heterodimers that are substantially free from both IL-17A and IL-17F homodimers. For example, IL-17A may be tagged with an epitope such as FLAG or myc epitope, while IL-17F is concurrently tagged with an epitope such as His or GST epitope, and both proteins simultaneously expressed in a cell. Extracts from the recombinant host cell, or media in which the host cells are cultured, would be obtained and subjected to two-step affinity chromatography purification under nonreducing conditions. The first affinity column would bind one of the two different tags, e.g., a FLAG epitope fused to IL-17A (or a fragment of IL-17A), and therefore the wash from the first column would contain (predominantly) IL-17F homodimers and the eluent from the first column would contain both IL-17A/IL-17F heterodimers and IL-17A homodimers. The eluent from the first column would then be placed over a second affinity column that specifically binds the other of the two different tags, e.g., a His tag fused to IL-17F. Thus, the wash from the second column would contain IL-17A homodimers and the eluent from the second column would predominantly or exclusively contain IL-17A/IL-17F heterodimers (i.e., substantially free of both IL-17A and IL-17F homodimers). The extracts from the recombinant host cells or the host cell media could be obtained under nonreducing conditions such that protein-protein interactions are not interrupted, or could be obtained under reducing conditions and then treated to allow proper refolding and interactions of the IL-17F and IL-17A monomers contained therein. One skilled in the art would readily realize that a host cell need not express both IL-17F and IL-17A fusion proteins; rather cell or media extracts from single transfectants, e.g., a host cell expressing either a IL-17A or IL-17F fusion protein, could be obtained and combined under conditions that allow the IL-17A and IL-17F monomers to dimerize.

The polypeptides related to the present invention, including IL-17F signaling antagonists, may also be produced by known conventional chemical synthesis. Methods for chemically synthesizing such polypeptides are well known to those skilled in the art. Such chemically synthetic polypeptides may possess biological properties in common with the natural, purified polypeptides, and thus may be employed as biologically active or immunological substitutes for the natural polypeptides.

The inventors were also able to isolate the “natural”, i.e., nonrecombinant form, of the polypeptides of the invention, including a natural form of IL-17A (see, e.g., Example 5). Thus, the polypeptides of the present invention include natural IL-17A homodimer, IL-17F homodimer, IL-17A/IL-17F heterodimer, etc.

The polypeptides related to the present invention, including IL-17F signaling antagonists, also encompass molecules that are structurally different from the disclosed polypeptides (e.g., which have a slightly altered sequence), but have substantially the same biochemical properties as the disclosed polypeptides (e.g., are changed only in functionally nonessential amino acid residues). Such molecules include naturally occurring allelic variants and deliberately engineered variants containing alterations, substitutions, replacements, insertions, or deletions. Techniques for such alterations, substitutions, replacements, insertions, or deletions are well known to those skilled in the art. In some embodiments, the polypeptide moiety is provided as a variant polypeptide having mutations in the naturally occurring sequence (wild type) that results in a sequence more resistant to proteolysis (relative to the nonmutated sequence).

IL-17F (including IL-17F homodimers and IL-17A/IL-17F heterodimers), IL-17R, IL-17RC polypeptides, fragments and/or fusion polypeptides thereof, recombinant and natural forms thereof, and/or natural IL-17A may be used to screen agents (e.g., other IL-17F signaling antagonists, e.g., anti-IL-17F antibodies) that are capable of binding IL-17F and/or inhibiting IL-17F bioactivity. Binding assays utilizing a desired binding protein, immobilized or not, are well known in the art and may be used for this purpose with the polypeptides related to the present invention, including the IL-17F signaling antagonists of the invention, e.g., IL-17R and/or IL-17RC. Purified cell-based or protein-based (cell-free) screening assays may be used to identify such agents. For example, IL-17F protein may be immobilized in purified form on a carrier and binding of potential ligands to purified IL-17F may be measured.

### Antibodies

In other embodiments, the invention provides IL-17F signaling antagonists as antibodies, i.e., intact antibodies and antigen binding fragments thereof, that specifically bind to IL-17F (including IL-17F homodimers and/or IL-17A/IL-17F heterodimers), preferably mammalian (e.g., human) IL-17F, or to the receptors for IL-17F, e.g., IL-17R and/or IL-17RC. In one embodiment, the antibodies are inhibitory antibodies, i.e., they inhibit at least one IL-17F bioactivity (e.g., binding of IL-17F and its receptor, IL-17F-mediated activation of signaling components (e.g., NF-κB), IL-17F-mediated induction of cytokine production, IL-17F-mediated increase in Aggrecanase etc.) and may be useful in diagnosing, prognosing, monitoring and/or treating disorders related to IL-17F signaling. The upregulation of IL-17A and IL-17F production by IL-21 (see Example 5) suggests that the proinflammatory effects associated with IL-21 binding to and activating IL-21R (e.g., IL-21 signaling) are mediated by IL-17A homodimer, IL-17F homodimer, and/or IL-17A/IL-17F heterodimer. Consequently, the antibodies of the invention that mitigate IL-17F signaling may also be inhibitory antibodies to at least one activity associated with IL-21 signaling (e.g., modulation of cytokine production, inflammation in inflammatory/autoimmune disorders (such as inflammatory bowel disorders or diseases (IBDs), rheumatoid arthritis, transplant/graft rejection, and psoriasis), etc.; see U.S. Patent Application Nos. 60/599,086 and 60/639,176) and may be useful in diagnosing, prognosing, monitoring and/or treating disorders associated with IL-21 signaling.

Additionally, the invention provides anti-IL-17F antibodies that specifically bind to but do not inhibit IL-17F signaling (i.e., detecting antibodies); such antibodies may be used to detect the presence of IL-17F protein (e.g., as a homodimer and/or heterodimer), e.g., as part of a kit for diagnosing, prognosing, and/or monitoring a disorder(s) related to IL-17F signaling. In one embodiment, the antibody is directed to IL-17F. In another embodiment, the antibody is a monoclonal or single specificity antibody. The antibodies may also be human, humanized, chimeric, or in vitro-generated antibodies against human IL-17F.

One of skill in the art will recognize that, as used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDRs”), interspersed with regions that are more conserved, termed “framework regions” (“FR”). The extent of the FRs and CDRs has been precisely defined (see, Kabat et al. (1991) Sequences of Proteins of Immunological Interest, Fifth Edition, U.S. Department of Health and Human Services, NIH Publication No. 91-3242; and Chothia et al. (1987) J. Mol. Biol. 196:901-917, which are hereby incorporated by reference). Each VH and VL is composed of three CDRs and four FRs, arranged from amino-terminus to carboxy-terminus in the following order: FR1, CDR1, FR2, CDR2, FR3, CDR3, FR4.

The antibody may further include a heavy and light chain constant region to thereby form a heavy and light immunoglobulin chain, respectively. In one embodiment, the antibody is a tetramer of two heavy immunoglobulin chains and two light immunoglobulin chains, wherein the heavy and light immunoglobulin chains are interconnected, e.g., by disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system.

Immunoglobulin refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd, or 214 amino acids) are encoded by a variable region gene at the NH₂-terminus (about 110 amino acids) and a kappa or lambda constant region gene at the COOH-terminus. Full-length immunoglobulin “heavy chains” (about 50 Kd, or 446 amino acids), are similarly encoded by a variable region gene (about 116 amino acids) and one of the other aforementioned constant region genes, e.g., gamma (encoding about 330 amino acids). The immunoglobulin heavy chain constant region genes encode for the antibody class, i.e., isotype (e.g., IgM or IgG1). The antigen binding fragment of an antibody (or simply “antibody portion,” or “fragment”), as used herein, refers to one or more fragments of a full-length antibody that retain the ability to specifically bind to an antigen (e.g., CD3). Examples of binding fragments encompassed within the term “antigen binding fragment” of an antibody include, but are not limited to, (i) an Fab fragment, a monovalent fragment consisting of the VL, VH, CL and CH1 domains; (ii) an F(ab′)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which consists of a VH domain; and (vi) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain Fv (scFv)). Such single chain antibodies are also intended to be encompassed within the term “antigen binding fragment” of an antibody. These antibody fragments are obtained using conventional techniques known to those skilled in the art, and the fragments are screened for utility in the same manner as are intact antibodies.

Antibody molecules to the polypeptides of the present invention, e.g., antibodies to IL-17F protein, IL-17R, and/or IL-17RC, may be produced by methods well known to those skilled in the art. For example, monoclonal antibodies may be produced by generation of hybridomas in accordance with known methods. Hybridomas formed in this manner are then screened using standard methods, such as an enzyme-linked immunosorbent assay (ELISA), to identify one or more hybridomas that produce an antibody that specifically binds with the polypeptides of the present invention. For example, IL-17F proteins of the invention may also be used to immunize animals to obtain polyclonal and monoclonal antibodies that react with the IL-17F protein and which may inhibit binding of IL-17F (e.g., IL-17F homodimer and/or IL-17A/IL-17F heterodimer) to its receptor, e.g., IL-17R or IL-17RC. Similarly, IL-17R or IL-17RC proteins may be used to obtain polyclonal and monoclonal antibodies that specifically react with IL-17R or IL-17RC, respectively, and which may inhibit binding of these receptors to IL-17F protein specifically (including IL-17F homodimer and IL-17A/IL-17F heterodimer), i.e., these antibodies do not inhibit binding of either or both of these receptors to other IL-17F family members, e.g., IL-17A homodimer. The peptide immunogens additionally may contain a cysteine residue at the carboxyl terminus, and may be conjugated to a hapten such as keyhole limpet hemocyanin (KLH). Additional peptide immunogens may be generated by replacing tyrosine residues with sulfated tyrosine residues. Methods for synthesizing such peptides are well known in the art. A full-length polypeptide of the present invention may be used as the immunogen, or, alternatively, antigenic peptide fragments of the polypeptides may be used. An antigenic peptide of a polypeptide of the present invention comprises at least 7 continuous amino acid residues and encompasses an epitope such that an antibody raised against the peptide forms a specific immune complex with the polypeptide. Preferably, the antigenic peptide comprises at least 10 amino acid residues, more preferably at least 15 amino acid residues, even more preferably at least 20 amino acid residues, and most preferably at least 30 amino acid residues.

Monoclonal antibodies may be generated by other methods known to those skilled in the art of recombinant DNA technology. As an alternative to preparing monoclonal antibody-secreting hybridomas, a monoclonal antibody to a polypeptide of the present invention may be identified and isolated by screening a recombinant combinatorial immunoglobulin library (e.g., an antibody phage display library) with a polypeptide related to the present invention (e.g., IL-17F, IL-17R, IL-17RC) to thereby isolate immunoglobulin library members that bind to the polypeptides related to the present invention (e.g., IL-17F, IL-17R, IL-17RC, respectively). Techniques and commercially available kits for generating and screening phage display libraries are well known to those skilled in the art. Additionally, examples of methods and reagents particularly amenable for use in generating and screening antibody display libraries can be found in the literature. For example, the “combinatorial antibody display” method is well known and was developed to identify and isolate antibody fragments having a particular antigen specificity, and can be utilized to produce monoclonal antibodies. After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primers to a conserved 3′ constant region, can be used for PCR amplification of the heavy and light chain variable regions from a number of murine antibodies; a similar strategy has also been used to amplify human heavy and light chain variable regions from human antibodies.

Polyclonal sera and antibodies may be produced by immunizing a suitable subject with a polypeptide of the present invention. The antibody titer in the immunized subject may be monitored over time by standard techniques, such as with ELISA using immobilized protein. If desired, the antibody molecules directed against a polypeptide of the present invention may be isolated from the subject or culture media and further purified by well-known techniques, such as protein A chromatography, to obtain an IgG fraction.

Fragments of antibodies to the polypeptides of the present invention may be produced by cleavage of the antibodies in accordance with methods well known in the art. For example, immunologically active Fab and F(ab′)₂ fragments may be generated by treating the antibodies with an enzyme such as pepsin.

Additionally, chimeric, humanized, and single-chain antibodies to the polypeptides of the present invention, comprising both human and nonhuman portions, may be produced using standard recombinant DNA techniques and/or a recombinant combinatorial immunoglobulin library. Humanized antibodies may also be produced using transgenic mice which are incapable of expressing endogenous immunoglobulin heavy and light chain genes, but which can express human heavy and light chain genes. For example, human monoclonal antibodies (mAbs) directed against, e.g., IL-17F protein, may be generated using transgenic mice carrying the human immunoglobulin genes rather than murine immunoglobulin genes. Splenocytes from these transgenic mice immunized with the antigen of interest may then be used to produce hybridomas that secrete human mAbs with specific affinities for epitopes from a human protein.

Chimeric antibodies, including chimeric immunoglobulin chains, may be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted.

An antibody or an immunoglobulin chain may be humanized by methods known in the art. Humanized antibodies, including humanized immunoglobulin chains, may be generated by replacing sequences of the Fv variable region that are not directly involved in antigen binding with equivalent sequences from human Fv variable regions. General methods for generating humanized antibodies are provided by Morrison (1985) Science 229:1202-07; Oi et al. (1986) BioTechniques 4:214; Queen et al., U.S. Pat. Nos. 5,585,089; 5,693,761; 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid sequences are well known to those skilled in the art and, for example, may be obtained from a hybridoma producing an antibody against a predetermined target. The recombinant DNA encoding the humanized antibody, or fragment thereof, then can be cloned into an appropriate expression vector.

Humanized or CDR-grafted antibody molecules or immunoglobulins may be produced by CDR grafting or CDR substitution, wherein one, two, or all CDRs of an immunoglobulin chain can be replaced. See, e.g., U.S. Pat. No. 5,225,539; Jones et al. (1986) Nature 321:552-25; Verhoeyan et al. (1988) Science 239:1534; Beidler et al. (1988) J. Immunol. 141:4053-60; Winter, U.S. Pat. No. 5,225,539, the contents of all of which are hereby incorporated by reference. Winter describes a CDR-grafting method that may be used to prepare the humanized antibodies of the present invention (UK Patent Application GB 2188638A; Winter, U.S. Pat. No. 5,225,539), the contents of which are hereby incorporated by reference. All of the CDRs of a particular human antibody may be replaced with at least a portion of a nonhuman CDR, or only some of the CDRs may be replaced with nonhuman CDRs. It is only necessary to replace the number of CDRs required for binding of the humanized antibody to a predetermined antigen.

Human antibodies may additionally be produced using transgenic nonhuman animals that are modified so as to produce fully human antibodies rather than the animal's endogenous antibodies in response to challenge by an antigen. See, e.g., PCT publication WO 94/02602. The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the XENOMOUSE™ as disclosed in PCT publications WO 96/33735 and WO 96/34096. This animal produces B cells that secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

Monoclonal, chimeric and humanized antibodies that have been modified by, e.g., deleting, adding, or substituting other portions of the antibody, e.g., the constant region, are also within the scope of the invention. As nonlimiting examples, an antibody can be modified by deleting the constant region, by replacing the constant region with another constant region, e.g., a constant region meant to increase half-life, stability, or affinity of the antibody, or a constant region from another species or antibody class, and by modifying one or more amino acids in the constant region to alter, for example, the number of glycosylation sites, effector cell function, Fc receptor (FcR) binding, complement fixation, etc.

Methods for altering an antibody constant region are known in the art. Antibodies with altered function, e.g., altered affinity for an effector ligand, such as FcR on a cell, or the C1 component of complement, can be produced by replacing at least one amino acid residue in the constant portion of the antibody with a different residue (see, e.g., EP 388,151 A1, U.S. Pat. No. 5,624,821 and U.S. Pat. No. 5,648,260, the contents of all of which are hereby incorporated by reference). Similar types of alterations to the murine (or other species) immunoglobulin may be applied to reduce or eliminate these functions, and are known in the art.

For example, it is possible to alter the affinity of an Fc region of an antibody (e.g., an IgG, such as a human IgG) for an FcR (e.g., Fc gamma R1), or for C1q binding by replacing the specified residue(s) with a residue(s) having an appropriate functionality on its side chain, or by introducing a charged functional group, such as glutamate or aspartate, or an aromatic nonpolar residue such as phenylalanine, tyrosine, tryptophan or alanine (see, e.g., U.S. Pat. No. 5,624,821).

Anti-IL-17F, anti-IL-17R, or anti-IL-17RC antibodies of the invention may be useful for isolating, purifying, and/or detecting IL-17F protein (e.g., in monomer, homodimer, or heterodimer formation), IL-17R, or IL-17RC polypeptides, respectively, in supernatant, cellular lysate, or on the cell surface. Antibodies disclosed in this invention may be also used diagnostically to monitor, e.g., IL-17F protein levels, as part of a clinical testing procedure, or clinically to target a therapeutic modulator to a cell or tissue comprising the antigen of the antibody. For example, a therapeutic such as a small molecule, or other therapeutic of the invention may be linked to an anti-IL-17F, anti-IL-17R, or anti-IL17RC antibody in order to target the therapeutic to the cell or tissue expressing IL-17F, IL-17R, or IL-17RC, respectively. Antagonistic antibodies (preferably monoclonal antibodies) that bind to IL-17F, IL-17R, or IL-17RC protein may also be useful in the treatment of a disease(s) related to IL-17F signaling, and/or a disease(s) associated with IL-21 signaling (e.g., IL-21 binding to and activation of IL-21R), due to the relationship between IL-21 and IL-17F production. Thus, the present invention further provides compositions comprising an inhibitory antibody that specifically binds to IL-17F (in monomeric and/or dimerized forms), IL-17R, or IL-17RC that decreases, limits, blocks, or otherwise reduces IL-17F signaling. Similarly, anti-IL-17F, anti-IL-17R, or anti-IL-17RC antibodies may be useful in isolating, purifying, detecting, and/or diagnostically monitoring IL-17F, IL-17R, or IL-17RC, respectively, and/or clinically targeting a therapeutic modulator to a cell or tissue comprising IL-17F, IL-17R, or IL-17RC, respectively.

In addition to antibodies for use in the instant invention, antibody-based molecules may also be employed to modulate the activity of IL-17F homodimers, IL-17A homodimers, and/or IL-17F/IL-17A homodimers. Such antibody-based molecules include small modular immunopharmaceutical (SMIP™) drugs (Trubion Pharmaceuticals, Seattle, Wash.). SMIPs are single-chain polypeptides composed of a binding domain for a cognate structure such as an antigen, a counterreceptor or the like, a hinge-region polypeptide having either one or no cysteine residues, and immunoglobulin CH2 and CH3 domains (see also www.trubion.com). SMIPs exhibit the binding specificity and activity of monoclonal antibodies, but are approximately one-third to one-half the size of conventional therapeutic monoclonal antibodies, and have an extensive in vivo half-life. SMIPs and their uses and applications are disclosed in, e.g., U.S. Published Patent Appln. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

### Screening Assays

The polynucleotides and polypeptides related to IL-17F signaling may be used in screening assays to identify pharmacological agents or lead compounds for agents that are capable of modulating the activity of IL-17F in a cell or organism and are thereby potential regulators of inflammatory responses. For example, samples containing IL-17F (either natural or recombinant) may be contacted with one of a plurality of test compounds (either biological agents or small organic molecules), and the biological activity of IL-17F in each of the treated samples can be compared with the biological activity of IL-17F in untreated samples or in samples contacted with different test compounds. Such comparisons will determine whether any of the test compounds results in: 1) a substantially decreased level of expression or biological activity of IL-17F, thereby indicating an antagonist of IL-17F, or 2) a substantially increased level of expression or biological activity of IL-17F, thereby indicating an agonist of IL-17F. In one embodiment, the identification of test compounds capable of modulating IL-17F activity is performed using high-throughput screening assays, such as BIACORE® (Biacore International AB, Uppsala, Sweden), BRET (bioluminescence resonance energy transfer), and FRET (fluorescence resonance energy transfer) assays, as well as ELISA and cell-based assays.

### Small Molecules

Decreased IL-17F activity (and/or at least one activity associated with IL-21 binding to and activation of IL-21R) in an organism (or subject) afflicted with (or at risk for) disorders related to IL-17F signaling (and/or disorders associated with IL-21 binding to and activation of IL-21R), e.g., inflammatory bowel disease, rheumatoid arthritis, transplant rejection, psoriasis, etc., or in a cell from such an organism (or subject) involved in such disorders, may also be achieved through the use of small molecules (usually organic small molecules) that antagonize, i.e., inhibit the activity of, IL-17F. Novel antagonistic small molecules may be identified by the screening methods described above and may be used in the treatment methods of the present invention described herein.

Conversely, increased IL-17F activity (and/or IL-21 associated activity) in an organism (or subject) afflicted with (or at risk for) an immune deficiency, e.g., neutropenia, or in a cell from such an organism (or subject) involved in such a disorder, may also be achieved through the use of small molecules (usually organic small molecules) that agonize, i.e., enhance the activity of, IL-17F. Novel agonistic small molecules may be identified by the screening methods described above and may be used in the methods of treating immune deficiencies, e.g., as described in U.S. Pat. Nos. 5,707,829; 6,043,344; 6,074,849 and U.S. patent application Ser. No. 10/102,080, all of which are incorporated by reference in their entireties.

The term small molecule refers to compounds that are not macromolecules (see, e.g., Karp (2000) Bioinformatics Ontology 16:269-85; Verkman (2004) AJP-Cell Physiol. 286:465-74). Thus, small molecules are often considered those compounds that are, e.g., less than one thousand daltons (e.g., Voet and Voet, Biochemistry, 2^{nd} ed., ed. N. Rose, Wiley and Sons, New York, 14 (1995)). For example, Davis et al. (2005) Proc. Natl. Acad. Sci. USA 102:5981-86, use the phrase small molecule to indicate folates, methotrexate, and neuropeptides, while Halpin and Harbury (2004) PLos Biology 2:1022-30, use the phrase to indicate small molecule gene products, e.g., DNAs, RNAs and peptides. Examples of natural small molecules include, but are not limited to, cholesterols, neurotransmitters, and siRNAs; synthesized small molecules include, but are not limited to, various chemicals listed in numerous commercially available small molecule databases, e.g., FCD (Fine Chemicals Database), SMID (Small Molecule Interaction Database), ChEBI (Chemical Entities of Biological Interest), and CSD (Cambridge Structural Database) (see, e.g., Alfarano et al. (2005) Nuc. Acids Res. Database Issue 33:D416-24).

### Methods for Diagnosing, Prognosing, and Monitoring the Progress of Disorders Related to IL-17F Signaling

It is well known in the art that immunological mechanisms studied in animal models, particularly murine models, may be and often are, translatable to the human immune system. As such, although many of the Examples disclosed herein demonstrate the ability of IL-17F signaling antagonists to inhibit IL-17F bioactivities in animal models, in addition to human samples, the disclosed methods for diagnosing, prognosing, and monitoring disorders related to IL-17F signaling will be particularly useful for diagnosing, prognosing and monitoring such disorders in humans.

The present invention provides methods for diagnosing, prognosing, and monitoring the progress of disorders related to IL-17F signaling in a subject (e.g., that directly or indirectly involve increases in the bioactivity of IL-17F) by detecting an upregulation of IL-17F activity, e.g., by detecting the upregulation of IL-17F, including but not limited to the use of such methods in human subjects. Due to the direct relationship between IL-21 and IL-17F, the invention also provides methods for diagnosing, prognosing, and monitoring the progress of disorders associated with IL-21 binding to and activation of IL-21R in a subject (e.g., that directly or indirectly involve increases in the bioactivity of IL-21) by detecting an upregulation of IL-17F activity, e.g., by detecting the upregulation of IL-17F, including but not limited to the use of such methods in human subjects. These methods may be performed by utilizing prepackaged diagnostic kits comprising at least one of the group comprising an IL-17F, IL-17R, or IL-17RC polynucleotide or fragments thereof, an IL-17F, IL-17R, or IL-17RC polypeptide or fragments thereof (including fusion proteins thereof), or antibodies to an IL-17F, IL-17R, or IL-17RC polypeptide or derivatives thereof, or modulators of IL-17F, IL-17R, or IL-17RC polynucleotides and/or polypeptides as described herein, which may be conveniently used, for example, in a clinical setting. A skilled artisan will recognize that other indirect methods may be used to confirm the upregulation of, e.g., IL-17F, such as counting the number of immune cells, e.g., neutrophils.

“Diagnostic” or “diagnosing” means identifying the presence or absence of a pathologic condition. Diagnostic methods include detecting upregulation of IL-17F signaling (and/or IL-21 signaling) by determining a test amount of the gene products (e.g., mRNA, cDNA, or polypeptide, including fragments thereof) of IL-17F, IL-17R, and/or IL-17RC in a biological sample from a subject (human or nonhuman mammal), and comparing the test amount with a normal amount or range (i.e., an amount or range from an individual(s) known not to suffer from disorders related to IL-17F signaling). Although a particular diagnostic method may not provide a definitive diagnosis of disorders related IL-17F signaling, it suffices if the method provides a positive indication that aids in diagnosis.

The present invention also provides methods for prognosing such disorders by detecting the upregulation of IL-17F activity, e.g., by detecting upregulation of IL-17F, IL-17R, or IL-17RC. “Prognostic” or “prognosing” means predicting the probable development and/or severity of a pathologic condition. Prognostic methods include determining the test amount of a gene product of IL-17F, IL-17R, or IL-17RC in a biological sample from a subject, and comparing the test amount to a prognostic amount or range (i.e., an amount or range from individuals with varying severities of disorders related to IL-17F signaling and/or disorders associated with IL-21 signaling) for the gene product of IL-17F, IL-17R, or IL-17RC, respectively. Various amounts of the IL-17F, IL-17R, or IL-17RC gene product in a test sample are consistent with certain prognoses for disorders related to IL-17F signaling and/or disorders associated with IL-21 signaling. The detection of an amount of IL-17F, IL-17R, or IL-17RC gene product at a particular prognostic level provides a prognosis for the subject.

The present invention also provides methods for monitoring the progress or course of such disorders related to IL-17F signaling (and/or disorders associated with IL-21 signaling) by detecting the upregulation of IL-17F activity, e.g., by detecting upregulation of IL-17F, IL-17R, or IL-17RC. Monitoring methods include determining the test amounts of a gene product of IL-17F, IL-17R, or IL-17RC in biological samples taken from a subject at a first and second time, and comparing the amounts. A change in amount of an IL-17F, IL-17R, or IL-17RC gene product between the first and second times indicates a change in the course of IL-17F signaling-related disorders (and/or IL-21 signaling-associated disorders), with a decrease in amount indicating remission of such disorders, and an increase in amount indicating progression of such disorders. Such monitoring assays are also useful for evaluating the efficacy of a particular therapeutic intervention in patients being treated for autoimmune disorders.

Increased IL-17F signaling in methods outlined above may be detected in a variety of biological samples, including bodily fluids (e.g., whole blood, plasma, and urine), cells (e.g., whole cells, cell fractions, and cell extracts), and other tissues. Biological samples also include sections of tissue, such as biopsies and frozen sections taken for histological purposes. Preferred biological samples include blood, plasma, lymph, tissue biopsies, urine, CSF (cerebrospinal fluid), synovial fluid, and BAL (bronchoalveolar lavage). It will be appreciated that analysis of a biological sample need not necessarily require removal of cells or tissue from the subject. For example, appropriately labeled agents that bind IL-17F signaling gene products (e.g., antibodies, nucleic acids) can be administered to a subject and visualized (when bound to the target) using standard imaging technology (e.g., CAT, NMR (MRI), and PET).

In the diagnostic and prognostic assays of the present invention, the IL-17F, IL-17R, or IL-17RC gene product is detected and quantified to yield a test amount. The test amount is then compared with a normal amount or range. An amount significantly above the normal amount or range is a positive sign in the diagnosis of disorders related to IL-17F signaling (and/or disorders associated with IL-21 binding to and activation of IL-21R). Particular methods of detection and quantitation of IL-17F, IL-17R, or IL-17RC gene products are described below.

Normal amounts or baseline levels of IL-17F, IL-17R, or IL-17RC gene products may be determined for any particular sample type and population. Generally, baseline (normal) levels of IL-17F, IL-17R, or IL-17RC protein or mRNA are determined by measuring respective amounts of IL-17F, IL-17R, or IL-17RC protein or mRNA in a biological sample type from normal (i.e., healthy) subjects. Alternatively, normal values of IL-17F, IL-17R, or IL-17RC gene products may be determined by measuring the amount in healthy cells or tissues taken from the same subject from which the diseased (or possibly diseased) test cells or tissues were taken. The amount of IL-17F, IL-17R, or IL-17RC gene products (either the normal amount or the test amount) may be determined or expressed on a per cell, per total protein, or per volume basis. To determine the cell amount of a sample, one can measure the level of a constitutively expressed gene product or other gene product expressed at known levels in cells of the type from which the biological sample was taken.

It will be appreciated that the assay methods of the present invention do not necessarily require measurement of absolute values of IL-17F, IL-17R, or IL-17RC gene products because relative values are sufficient for many applications of these methods. It will also be appreciated that in addition to the quantity or abundance of IL-17F, IL-17R, or IL-17RC gene products, variant or abnormal IL-17F, IL-17R, or IL-17RC gene products or their expression patterns (e.g., mutated transcripts, truncated polypeptides) may be identified by comparison to normal gene products and expression patterns.

Whether the expression of a particular gene in two samples is significantly similar or significantly different, e.g., significantly above or significantly below a given level, depends on the gene itself and, inter alia, its variability in expression between different individuals or different samples. It is within the skill in the art to determine whether expression levels are significantly similar or different. Factors such as genetic variation, e.g., in IL-17F and/or IL-17A expression levels, between individuals, species, organs, tissues, or cells may be taken into consideration (when and where necessary) when determining whether the level of expression, e.g., of IL-17F and/or IL-17A, between two samples is significantly similar or significantly different, e.g., significantly above a given level. As a result of the natural heterogeneity in gene expression between individuals, species, organs, tissues, or cells, phrase such as “significantly similar” or “significantly above” cannot be defined as a precise percentage or value, but rather can be ascertained by one skilled in the art upon practicing the invention.

The diagnostic, prognostic, and monitoring assays of the present invention involve detecting and quantifying IL-17F, IL-17R, or IL-17RC gene products in biological samples. IL-17F, IL-17R, or IL-17RC gene products include mRNAs and polypeptides, and both can be measured using methods well known to those skilled in the art.

For example, mRNA can be directly detected and quantified using hybridization-based assays, such as Northern hybridization, in situ hybridization, dot and slot blots, and oligonucleotide arrays. Hybridization-based assays refer to assays in which a probe nucleic acid is hybridized to a target nucleic acid. In some formats, the target, the probe, or both are immobilized. The immobilized nucleic acid may be DNA, RNA, or another oligonucleotide or polynucleotide, and may comprise naturally or nonnaturally occurring nucleotides, nucleotide analogs, or backbones. Methods of selecting nucleic acid probe sequences for use in the present invention are based on the nucleic acid sequence of IL-17F, IL-17R, or IL-17RC and are well known in the art.

Alternatively, mRNA can be amplified before detection and quantitation. Such amplification-based assays are well known in the art and include polymerase chain reaction (PCR), reverse-transcription-PCR (RT-PCR), PCR-enzyme-linked immunosorbent assay (PCR-ELISA), and ligase chain reaction (LCR). Primers and probes for producing and detecting amplified IL-17F, IL-17R, or IL-17RC gene products (e.g., mRNA or cDNA) may be readily designed and produced without undue experimentation by those of skill in the art based on the nucleic acid sequences of IL-17F, IL-17R, or IL-17RC, respectively. As a nonlimiting example, amplified IL-17F gene products may be directly analyzed, for example, by gel electrophoresis; by hybridization to a probe nucleic acid; by sequencing; by detection of a fluorescent, phosphorescent, or radioactive signal; or by any of a variety of well-known methods. In addition, methods are known to those of skill in the art for increasing the signal produced by amplification of target nucleic acid sequences. One of skill in the art will recognize that whichever amplification method is used, a variety of quantitative methods known in the art (e.g., quantitative PCR) may be used if quantitation of gene products is desired.

IL-17F, IL-17R, or IL-17RC polypeptides (or fragments thereof) may be detected using various well-known immunological assays employing the respective anti-IL-17F, anti-IL-17R, or anti-IL-17RC antibodies that may be generated as described above. Immunological assays refer to assays that utilize an antibody (e.g., polyclonal, monoclonal, chimeric, humanized, scFv, and/or fragments thereof) that specifically binds to, e.g., an IL-17F polypeptide (or a fragment thereof). Such well-known immunological assays suitable for the practice of the present invention include ELISA, radioimmunoassay (RIA), immunoprecipitation, immunofluorescence, fluorescence-activated cell sorting (FACS), and Western blotting. An IL-17F polypeptide may also be detected using a labeled IL-17R and/or IL-17RC polypeptide(s). Conversely, IL-17R or IL-17RC may be detected using a labeled IL-17F polypeptide.

One of skill in the art will understand that the aforementioned methods may be applied to disorders related to IL-17F signaling.

### Uses of Molecules Related to IL-17F Signaling in Therapy

The present inventors are the first to demonstrate, inter alia, the following: 1) binding of IL-17R and/or IL-17RC by IL-17F, or other IL-17F agonists, is correlated with increased neutrophil infiltration, cartilage destruction, etc.; 2) antibodies directed toward IL-17F may be used to detect IL-17F protein and to inhibit at least one IL-17F bioactivity; 3) siRNAs directed to IL-17R and IL-17RC may be used to decrease IL-17A and IL-17F bioactivity; 4) IL-17F protein may form an IL-17F homodimer and an IL-17A/IL-17F heterodimer, and thus, inhibitory antibodies directed toward IL-17F may also inhibit IL-17A bioactivity that is mediated by IL-17A/IL-17F heterodimers; 5) IL-21 acts synergistically with CD28 to upregulate IL-17A homodimers, IL-17F homodimers, and IL-17A/IL-17F heterodimers, and thus antibodies that inhibit IL-17F activity (e.g., IL-17F homodimer activity and/or IL-17A/IL-17F heterodimer activity) may regulate IL-21 signal; 6) natural and recombinant IL-17A homodimers, IL-17F homodimers, and IL-17A/IL-17F heterodimers may be isolated and purified; 7) IL-17F heterodimers possess IL-17F bioactivity; 8) antibodies against human IL-17F cross react with and partially neutralize primate IL-17F; and 9) both IL-17F and IL-17A are increased in lesional tissues from human patients with psoriasis and involved tissues in human patients with Crohn's disease and ulcerative colitis. Although some animal models have been used to identify some of the above correlations, it is well known in the art that immunological mechanisms studied in animal models may be, and often are, translatable to the human immune system. Additionally, the antibodies of the invention were used to isolate natural IL-17F in its homodimeric and heterodimeric forms from primary human T cells, while the experiments involving IL-17F regulation of Aggrecanase and profiling the expression levels of IL-17A and IL-17F in psoriatic lesions and involved tissues in inflammatory bowel disease were undertaken in human cells and tissues. As such, the disclosed methods for using molecules related to IL-17F signaling, e.g., IL-17F agonists or IL-17F signaling antagonists, to treat disorders related to IL-17F signaling and/or disorders associated with IL-21 signaling, will be particularly useful for treating such disorders in humans.

The IL-17F signaling-related molecules disclosed herein, including modulators of IL-17F, IL-17R, or IL-17RC polynucleotide and/or polypeptide activity identified using the methods described above, may be used in vitro, ex vivo, or incorporated into pharmaceutical compositions and administered to individuals in vivo to treat, for example, disorders related to IL-17F signaling and/or IL-21 signaling, by administration of an IL-17F signaling antagonist (e.g., IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17R, or anti-IL-17RC antibodies; and/or antagonistic small molecules, etc.). Several pharmacogenomic approaches to be considered in determining whether to administer IL-17F signaling-related molecules are well known to one of skill in the art and include genome-wide association, candidate gene approach, and gene expression profiling. A pharmaceutical composition of the invention is formulated to be compatible with its intended route of administration (e.g., oral compositions generally include an inert diluent or an edible carrier). Other nonlimiting examples of routes of administration include parenteral (e.g., intravenous), intradermal, subcutaneous, oral (e.g., inhalation), transdermal (topical), transmucosal, and rectal administration. The pharmaceutical compositions compatible with each intended route are well known in the art.

IL-17F agonists or IL-17F signaling antagonists may be used as pharmaceutical compositions when combined with a pharmaceutically acceptable carrier. Such a composition may contain, in addition to an IL-17F signaling-related molecules (e.g., IL-17F agonists or IL-17F signaling antagonists) and carrier, various diluents, fillers, salts, buffers, stabilizers, solubilizers, and other materials well known in the art. The term “pharmaceutically acceptable” means a nontoxic material that does not interfere with the effectiveness of the biological activity of the active ingredient(s). The characteristics of the carrier will depend on the route of administration.

The pharmaceutical composition of the invention may also contain cytokines, lymphokines, or other hematopoietic factors such as M-CSF, GM-CSF, IL-1, IL-2, IL-3, IL-4, IL-5, IL-6, IL-7, IL-8, IL-9, IL-10, IL-11, IL-12, IL-14, IL-15, G-CSF, stem cell factor, and erythropoietin. The pharmaceutical composition may also include anticytokine antibodies as described in more detail below. The pharmaceutical composition may contain thrombolytic or antithrombotic factors such as plasminogen activator and Factor VIII. The pharmaceutical composition may further contain other anti-inflammatory agents as described in more detail below. Such additional factors and/or agents may be included in the pharmaceutical composition to produce a synergistic effect with IL-17F agonists or IL-17F signaling antagonists, or to minimize side effects caused by the IL-17F agonists or IL-17F signaling antagonists. Conversely IL-17F agonists or IL-17F signaling antagonists may be included in formulations of the particular cytokine, lymphokine, other hematopoietic factor, thrombolytic or antithrombotic factor, or anti-inflammatory agent to minimize side effects of the cytokine, lymphokine, other hematopoietic factor, thrombolytic or antithrombotic factor, or anti-inflammatory agent.

The pharmaceutical composition of the invention may be in the form of a liposome in which IL-17F agonists or IL-17F signaling antagonists are combined, in addition to other pharmaceutically acceptable carriers, with amphipathic agents such as lipids that exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, etc.

As used herein, the term “therapeutically effective amount” means the total amount of each active component of the pharmaceutical composition or method that is sufficient to show a meaningful patient benefit, e.g., amelioration of symptoms of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of an IL-17F agonist or IL-17F signaling antagonist is administered to a subject, e.g., a mammal (e.g., a human). An IL-17F signaling-related molecule may be administered in accordance with the method of the invention either alone or in combination with other therapies, such as treatments employing cytokines, lymphokines or other hematopoietic factors, or anti-inflammatory agents. When coadministered with one or more agents, IL-17F signaling antagonists may be administered either simultaneously with the second agent, or sequentially. If administered sequentially, the attending physician will decide on the appropriate sequence of administering, e.g., an IL-17R and/or IL-17RC polypeptide (or fusion protein thereof) and/or inhibiting antibody in combination with other agents.

When a therapeutically effective amount of an IL-17F agonist or IL-17F signaling antagonist is administered orally, the binding agent will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% binding agent, and preferably from about 25 to 90% binding agent. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition contains from about 0.5 to 90% by weight of the binding agent, and preferably from about 1 to 50% by weight of the binding agent.

When a therapeutically effective amount of an IL-17F agonist or IL-17F signaling antagonist is administered by intravenous, cutaneous or subcutaneous injection, the IL-17F agonist or IL-17F signaling antagonist will be in the form of a pyrogen-free, parenterally acceptable aqueous solution. The preparation of such parenterally acceptable protein solutions, having due regard to pH, isotonicity, stability, and the like, is within the skill of those in the art. A preferred pharmaceutical composition for intravenous, cutaneous, or subcutaneous injection should contain, in addition to the IL-17F agonist or IL-17F signaling antagonist, an isotonic vehicle such as sodium chloride injection, Ringer's injection, dextrose injection, dextrose and sodium chloride injection, lactated Ringer's injection, or other vehicle as known in the art. The pharmaceutical composition of the present invention may also contain stabilizers, preservatives, buffers, antioxidants, or other additive known to those of skill in the art.

The amount of an IL-17F agonist or IL-17F signaling antagonist in the pharmaceutical composition of the present invention will depend upon the nature and severity of the condition being treated, and on the nature of prior treatments that the patient has undergone. Ultimately, the attending physician will decide the amount of IL-17F agonist or IL-17F signaling antagonist with which to treat each individual patient. Initially, the attending physician will administer low doses of IL-17F agonist or IL-17F signaling antagonist and observe the patient's response. Larger doses of IL-17F agonist or IL-17F signaling antagonist may be administered until the optimal therapeutic effect is obtained for the patient, and at that point the dosage is not generally increased further. It is contemplated that the various pharmaceutical compositions used to practice the method of the present invention should contain about 0.1 μg to about 100 mg of IL-17F agonist or IL-17F signaling antagonist, e.g., IL-17R and/or IL-17RC (including fusion proteins thereof), per kg body weight.

The duration of intravenous (i.v.) therapy using a pharmaceutical composition of the present invention will vary, depending on the severity of the disease being treated and the condition and potential idiosyncratic response of each individual patient. It is contemplated that the duration of each application of the IL-17F agonist or IL-17F signaling antagonist may be in the range of 12 to 24 hours of continuous i.v. administration. Also contemplated is subcutaneous (s.c.) therapy using a pharmaceutical composition of the present invention. These therapies can be administered daily, weekly, or, more preferably, biweekly, or monthly. It is also contemplated that where the IL-17F agonist or IL-17F signaling antagonist is a small molecule (e.g., for oral delivery), the therapies may be administered daily, twice a day, three times a day, etc. Ultimately the attending physician will decide on the appropriate duration of i.v. or s.c. therapy, or therapy with a small molecule, and the timing of administration of the therapy, using the pharmaceutical composition of the present invention.

The polynucleotides and proteins of the present invention are expected to exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

### Uses of IL-17F Signaling Antagonists to Decrease Inflammation

In one aspect, the invention features a method of decreasing an inflammatory response, e.g., due to IL-21 signaling. The method may comprise contacting a population of cells with an IL-17F signaling antagonist (e.g., IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17R, or IL-17RC antibodies; and/or antagonistic small molecules, etc.) in an amount sufficient to inhibit the IL-17F activity of the cell or population. Antagonists to IL-17F signaling may also be administered to subjects for whom suppression of IL-17F signaling (and/or IL-21 signaling) is desired. These conditions include, but are not limited to, inflammatory disorders, e.g., autoimmune diseases (e.g., arthritis (including rheumatoid arthritis), psoriasis, systemic lupus erythematosus, multiple sclerosis), respiratory diseases (e.g., COPD, cystic fibrosis, asthma, allergy), transplant rejection (including solid organ transplant rejection), and inflammatory bowel diseases (e.g., ulcerative colitis, Crohn's disease).

These methods are based, at least in part, on the finding that interfering with IL-17F signaling, e.g., by using an interfering anti-IL-17F antibody, decreases IL-17F-associated inflammatory responses, e.g., cytokine production by primary fibroblast-like synoviocytes (Example 4.2). Accordingly, IL-17F signaling antagonists, i.e., molecules that inhibit IL-17F activity (e.g., anti-IL-17F antibodies) may be used to decrease inflammation in vivo, e.g., for treating or preventing disorders related to IL-17F signaling and/or disorders related to IL-21 signaling.

The methods of using IL-17F signaling antagonists may also be used inhibit IL-17F inflammatory activity and thus, can be used to treat or prevent a variety of immune disorders. Nonlimiting examples of the disorders that can be treated or prevented include, but are not limited to, transplant rejection, autoimmune diseases (including, for example, diabetes mellitus, arthritis (including rheumatoid arthritis, juvenile rheumatoid arthritis, osteoarthritis, psoriatic arthritis, reactive arthritis), multiple sclerosis, encephalomyelitis, myasthenia gravis, systemic lupus erythematosus (SLE), autoimmune thyroiditis, dermatitis (including atopic dermatitis and eczematous dermatitis), Reiter's syndrome, psoriasis, Sjögren's syndrome, Crohn's disease, aphthous ulcer, iritis, conjunctivitis, keratoconjunctivitis, ulcerative colitis, spondyloarthropathy, ankylosing spondylitis, intrinsic asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, pulmonary exacerbation (e.g., due to bacterial infection), and allergy, such as atopic allergy. Preferred disorders that can be treated using methods which comprise the administration of IL-17F signaling antagonists, e.g., an inhibitory IL-17F antibody, include, but are not limited to, inflammatory disorders, e.g., autoimmune diseases (e.g., arthritis (including rheumatoid arthritis), psoriasis, systemic lupus erythematosus, multiple sclerosis), respiratory diseases (e.g., COPD, cystic fibrosis, asthma, allergy), transplant rejection (including solid organ transplant rejection), and inflammatory bowel diseases (e.g., ulcerative colitis, Crohn's disease).

Using IL-17F signaling antagonists (e.g., IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17R, or IL-17RC antibodies; and/or antagonistic small molecules, etc.), it is possible to modulate immune responses in a number of ways. Downregulation may be in the form of inhibiting or blocking an inflammatory response already in progress, or may involve preventing the induction of an inflammatory response.

In one embodiment, IL-17F signaling antagonists, including pharmaceutical compositions thereof, are administered in combination therapy, i.e., combined with other agents, e.g., therapeutic agents, that are useful for treating pathological conditions or disorders, such as immune disorders and inflammatory diseases. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

For example, the combination therapy can include one or more IL-17F signaling antagonists (e.g., IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17R, or IL-17RC antibodies; and/or antagonistic small molecules, etc.) coformulated with, and/or coadministered with, one or more additional therapeutic agents, e.g., one or more cytokine and growth factor inhibitors, immunosuppressants, anti-inflammatory agents, metabolic inhibitors, enzyme inhibitors, and/or cytotoxic or cytostatic agents, as described in more detail below. Furthermore, one or more IL-17F signaling antagonists described herein may be used in combination with two or more of the therapeutic agents described herein. Such combination therapies may advantageously utilize lower dosages of the administered therapeutic agents, thus avoiding possible toxicities or complications associated with the various monotherapies. Moreover, the therapeutic agents disclosed herein act on pathways that differ from the IL-17F receptor signaling pathway, and thus are expected to enhance and/or synergize with the effects of the IL-17F signaling antagonists.

Preferred therapeutic agents used in combination with an IL-17F signaling antagonist are those agents that interfere at different stages in an inflammatory response. In one embodiment, one or more IL-17F signaling antagonists described herein may be coformulated with, and/or coadministered with, one or more additional agents such as other cytokine or growth factor antagonists (e.g., soluble receptors, peptide inhibitors, small molecules, ligand fusions); or antibodies or antigen binding fragments thereof that bind to other targets (e.g., antibodies that bind to other cytokines or growth factors, their receptors, or other cell surface molecules); and anti-inflammatory cytokines or agonists thereof. Nonlimiting examples of the agents that can be used in combination with the IL-17F signaling antagonists described herein, include, but are not limited to, antagonists of one or more interleukins (ILs) or their receptors, e.g., antagonists of IL-1, IL-2, IL-6, IL-7, IL-8, IL-12, IL-13, IL-15, IL-16, IL-18, IL-21 and IL-22; antagonists of cytokines or growth factors or their receptors, such as tumor necrosis factor (TNF), LT, EMAP-II, GM-CSF, FGF and PDGF. IL-17F signaling antagonists can also be combined with inhibitors of, e.g., antibodies to, cell surface molecules such as CD2, CD3, CD4, CD8, CD20 (e.g., the CD20 inhibitor rituximab (RITUXAN®)), CD25, CD28, CD30, CD40, CD45, CD69, CD80 (B7.1), CD86 (B7.2), CD90, or their ligands, including CD154 (gp39 or CD40L), or LFA-1/ICAM-1 and VLA-4/VCAM-1 (Yusuf-Makagiansar et al. (2002) Med. Res. Rev. 22:146-67). Preferred antagonists that can be used in combination with IL-17F signaling antagonists described herein include antagonists of IL-1, IL-12, TNFα, IL-15, IL-18, and IL-22.

Examples of those agents include IL-12 antagonists, such as chimeric, humanized, human or in vitro-generated antibodies (or antigen binding fragments thereof) that bind to IL-12 (preferably human IL-12), e.g., the antibody disclosed in WO 00/56772; IL-12 receptor inhibitors, e.g., antibodies to human IL-12 receptor; and soluble fragments of the IL-12 receptor, e.g., human IL-12 receptor. Examples of IL-15 antagonists include antibodies (or antigen binding fragments thereof) against IL-15 or its receptor, e.g., chimeric, humanized, human or in vitro-generated antibodies to human IL-15 or its receptor, soluble fragments of the IL-15 receptor, and IL-15-binding proteins. Examples of IL-18 antagonists include antibodies, e.g., chimeric, humanized, human or in vitro-generated antibodies (or antigen binding fragments thereof), to human IL-18, soluble fragments of the IL-18 receptor, and IL-18 binding proteins (IL-18BP). Examples of IL-1 antagonists include Interleukin-1-converting enzyme (ICE) inhibitors, such as Vx740, IL-1 antagonists, e.g., IL-1RA (anikinra, KINERET™, Amgen), sIL1RII (Immunex), and anti-IL-1 receptor antibodies (or antigen binding fragments thereof).

Examples of TNF antagonists include chimeric, humanized, human or in vitro-generated antibodies (or antigen binding fragments thereof) to TNF (e.g., human TNFα), such as (HUMIRA™, D2E7, human TNFα antibody), CDP-571/CDP-870/BAY-10-3356 (humanized anti-TNFα antibody; Celltech/Pharmacia), cA2 (chimeric anti-TNFα antibody; REMICADE®, Centocor); anti-TNF antibody fragments (e.g., CPD870); soluble fragments of the TNF receptors, e.g., p55 or p75 human TNF receptors or derivatives thereof, e.g., 75 kdTNFR-IgG (75 kD TNF receptor-IgG fusion protein, ENBREL™; Immunex), p55 kdTNFR-IgG (55 kD TNF receptor-IgG fusion protein (LENERCEPT®)); enzyme antagonists, e.g., TNFα converting enzyme (TACE) inhibitors (e.g., an alpha-sulfonyl hydroxamic acid derivative, and N-hydroxyformamide TACE inhibitor GW 3333, -005, or -022); and TNF-bp/s-TNFR (soluble TNF binding protein). Preferred TNF antagonists are soluble fragments of the TNF receptors, e.g., p55 or p75 human TNF receptors or derivatives thereof, e.g., 75 kdTNFR-IgG, and TNFα converting enzyme (TACE) inhibitors.

In other embodiments, the IL-17F signaling antagonists described herein may be administered in combination with one or more of the following: IL-13 antagonists, e.g., soluble IL-13 receptors (sIL-13) and/or antibodies against IL-13; IL-2 antagonists, e.g., DAB 486-IL-2 and/or DAB 389-IL-2 (IL-2 fusion proteins, Seragen), and/or antibodies to IL-2R, e.g., anti-Tac (humanized anti-IL-2R, Protein Design Labs). Yet another combination includes IL-17F signaling antagonists (e.g., IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17R, or IL-17RC antibodies; and/or antagonistic small molecules, etc.), antagonistic small molecules, and/or inhibitory antibodies in combination with nondepleting anti-CD4 inhibitors (IDEC-CE9.1/SB 210396; nondepleting primatized anti-CD4 antibody; IDEC/SmithKline) Yet other preferred combinations include antagonists of the costimulatory pathway CD80 (B7.1) or CD86 (B7.2), including antibodies, soluble receptors or antagonistic ligands; as well as p-selectin glycoprotein ligand (PSGL), anti-inflammatory cytokines, e.g., IL-4 (DNAX/Schering); IL-10 (SCH 52000; recombinant IL-10 DNAX/Schering); IL-13 and TGF-β, and agonists thereof (e.g., agonist antibodies).

In other embodiments, one or more IL-17F signaling antagonists can be coformulated with, and/or coadministered with, one or more anti-inflammatory drugs, immunosuppressants, or metabolic or enzymatic inhibitors. Nonlimiting examples of the drugs or inhibitors that can be used in combination with the IL-17F signaling antagonists (e.g., IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17R, or IL-17RC antibodies; and/or antagonistic small molecules, etc.) described herein, include, but are not limited to, one or more of: nonsteroidal anti-inflammatory drug(s) (NSAIDs), e.g., ibuprofen, tenidap, naproxen, meloxicam, piroxicam, diclofenac, and indomethacin; sulfasalazine; corticosteroids such as prednisolone; cytokine suppressive anti-inflammatory drug(s) (CSAIDs); inhibitors of nucleotide biosynthesis, e.g., inhibitors of purine biosynthesis, folate antagonists (e.g., methotrexate (N-[4-[[(2,4-diamino-6-pteridinyl)methyl]methylamino]benzoyl]-L-glutamic acid); and inhibitors of pyrimidine biosynthesis, e.g., dihydroorotate dehydrogenase (DHODH) inhibitors. Preferred therapeutic agents for use in combination with IL-17F signaling antagonists include NSAIDs, CSAIDs, (DHODH) inhibitors (e.g., leflunomide), and folate antagonists (e.g., methotrexate).

Examples of additional inhibitors include one or more of: corticosteroids (oral, inhaled and local injection); immunosuppresants, e.g., cyclosporin, tacrolimus (FK-506); and mTOR inhibitors, e.g., sirolimus (rapamycin—RAPAMUNE™ or rapamycin derivatives, e.g., soluble rapamycin derivatives (e.g., ester rapamycin derivatives, e.g., CCI-779); agents which interfere with signaling by proinflammatory cytokines such as TNFα or IL-1 (e.g. IRAK, NIK, IKK, p38 or MAP kinase inhibitors); COX2 inhibitors, e.g., celecoxib, rofecoxib, and variants thereof; phosphodiesterase inhibitors, e.g., R973401 (phosphodiesterase Type IV inhibitor); phospholipase inhibitors, e.g., inhibitors of cytosolic phospholipase 2 (cPLA2) (e.g., trifluoromethyl ketone analogs); inhibitors of vascular endothelial cell growth factor or growth factor receptor, e.g., VEGF inhibitor and/or VEGF-R inhibitor; and inhibitors of angiogenesis. Preferred therapeutic agents for use in combination with IL-17F signaling antagonists (e.g., IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17R, or IL-17RC antibodies; and/or antagonistic small molecules, etc.) are immunosuppresants, e.g., cyclosporin, tacrolimus (FK-506); mTOR inhibitors, e.g., sirolimus (rapamycin) or rapamycin derivatives, e.g., soluble rapamycin derivatives (e.g., ester rapamycin derivatives, e.g., CCI-779); COX2 inhibitors, e.g., celecoxib and variants thereof; and phospholipase inhibitors, e.g., inhibitors of cytosolic phospholipase 2 (cPLA2), e.g., trifluoromethyl ketone analogs.

Additional examples of therapeutic agents that can be combined with an IL-17F signaling antagonist include one or more of: 6-mercaptopurines (6-MP); azathioprine sulphasalazine; mesalazine; olsalazine; chloroquine/hydroxychloroquine (PLAQUENIL); pencillamine; aurothiornalate (intramuscular and oral); azathioprine; colchicine; beta-2 adrenoreceptor agonists (salbutamol, terbutaline, salmeteral); xanthines (theophylline, aminophylline); cromoglycate; nedocromil; ketotifen; ipratropium and oxitropium; mycophenolate mofetil; adenosine agonists; antithrombotic agents; complement inhibitors; and adrenergic agents.

The use of the IL-17F signaling antagonists disclosed herein in combination with other therapeutic agents to treat or prevent specific disorders related to IL-17F signaling is discussed in further detail below.

Nonlimiting examples of agents for treating or preventing arthritic disorders (e.g., rheumatoid arthritis, inflammatory arthritis, rheumatoid arthritis, juvenile rheumatoid arthritis, osteoarthritis and psoriatic arthritis), with which IL-17F signaling antagonists may be combined include one or more of the following: IL-12 antagonists as described herein; NSAIDs; CSAIDs; TNFs, e.g., TNFα, antagonists as described herein; nondepleting anti-CD4 antibodies as described herein; IL-2 antagonists as described herein; anti-inflammatory cytokines, e.g., IL-4, IL-10, IL-13 and TGFα, or agonists thereof; IL-1 or IL-1 receptor antagonists as described herein; phosphodiesterase inhibitors as described herein; Cox-2 inhibitors as described herein; iloprost: methotrexate; thalidomide and thalidomide-related drugs (e.g., Celgen); leflunomide; inhibitor of plasminogen activation, e.g., tranexamic acid; cytokine inhibitor, e.g., T-614; prostaglandin E1; azathioprine; an inhibitor of interleukin-1 converting enzyme (ICE); zap-70 and/or lck inhibitor (inhibitor of the tyrosine kinase zap-70 or lck); an inhibitor of vascular endothelial cell growth factor or vascular endothelial cell growth factor receptor as described herein; an inhibitor of angiogenesis as described herein; corticosteroid anti-inflammatory drugs (e.g., SB203580); TNF-convertase inhibitors; IL-11; IL-13; IL-17 inhibitors; gold; penicillamine; chloroquine; hydroxychloroquine; chlorambucil; cyclophosphamide; cyclosporine; total lymphoid irradiation; antithymocyte globulin; CD5-toxins; orally administered peptides and collagen; lobenzarit disodium; cytokine regulating agents (CRAs) HP228 and HP466 (Houghten Pharmaceuticals, Inc.); ICAM-1 antisense phosphorothioate oligodeoxynucleotides (ISIS 2302; Isis Pharmaceuticals, Inc.); soluble complement receptor 1 (TP10; T Cell Sciences, Inc.); prednisone; orgotein; glycosaminoglycan polysulphate; minocycline (MINOCIN®); anti-IL2R antibodies; marine and botanical lipids (fish and plant seed fatty acids); auranofin; phenylbutazone; meclofenamic acid; flufenamic acid; intravenous immune globulin; zileuton; mycophenolic acid (RS-61443); tacrolimus (FK-506); sirolimus (rapamycin); amiprilose (therafectin); cladribine (2-chlorodeoxyadenosine); and azaribine. Preferred combinations include one or more IL-17F signaling antagonists (e.g., IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17R, or IL-17RC antibodies; and/or antagonistic small molecules, etc.) in combination with methotrexate or leflunomide, and in moderate or severe rheumatoid arthritis cases, cyclosporine.

Preferred examples of inhibitors to use in combination with IL-17F signaling antagonists to treat arthritic disorders include TNF antagonists (e.g., chimeric, humanized, human or in vitro-generated antibodies, or antigen binding fragments thereof, that bind to TNF; soluble fragments of a TNF receptor, e.g., p55 or p75 human TNF receptor or derivatives thereof, e.g., 75 kdTNFR-IgG (75 kD TNF receptor-IgG fusion protein, ENBREL™), p55 kD TNF receptor-IgG fusion protein; TNF enzyme antagonists, e.g., TNFα converting enzyme (TACE) inhibitors); antagonists of IL-12, IL-15, IL-18, IL-22; T cell and B cell-depleting agents (e.g., anti-CD4 or anti-CD22 antibodies); small molecule inhibitors, e.g., methotrexate and leflunomide; sirolimus (rapamycin) and analogs thereof, e.g., CCI-779; cox-2 and cPLA2 inhibitors; NSAIDs; p38 inhibitors, TPL-2, Mk-2 and NFkb inhibitors; RAGE or soluble RAGE; P-selectin or PSGL-1 inhibitors (e.g., small molecule inhibitors, antibodies thereto, e.g., antibodies to P-selectin); estrogen receptor beta (ERB) agonists or ERB-NFkb antagonists. Most preferred additional therapeutic agents that can be coadministered and/or coformulated with one or more IL-17F signaling antagonists (e.g., IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17R, or IL-17RC antibodies; and/or antagonistic small molecules, etc.) include one or more of: a soluble fragment of a TNF receptor, e.g., p55 or p75 human TNF receptor or derivatives thereof, e.g., 75 kdTNFR-IgG (75 kD TNF receptor-IgG fusion protein, ENBREL™); methotrexate, leflunomide, or a sirolimus (rapamycin) or an analog thereof, e.g., CCI-779.

Nonlimiting examples of agents for treating or preventing multiple sclerosis with which IL-17F signaling antagonists can be combined include the following: interferons, e.g., interferon-alphala (e.g., AVONEX™; Biogen) and interferon-1b (BETASERON™ Chiron/Berlex); Copolymer 1 (Cop-1; COPAXONE™ Teva Pharmaceutical Industries, Inc.); hyperbaric oxygen; intravenous immunoglobulin; cladribine; TNF antagonists as described herein; corticosteroids; prednisolone; methylprednisolone; azathioprine; cyclophosphamide; cyclosporine; cyclosporine A, methotrexate; 4-aminopyridine; and tizanidine. Additional antagonists that can be used in combination with IL-17F signaling antagonists include antibodies to or antagonists of other human cytokines or growth factors, for example, TNF, LT, IL-1, IL-2, IL-6, IL-7, IL-8, IL-12 IL-15, IL-16, IL-18, EMAP-11, GM-CSF, FGF, and PDGF. IL-17F signaling antagonists as described herein can be combined with antibodies to cell surface molecules such as CD2, CD3, CD4, CD8, CD25, CD28, CD30, CD40, CD45, CD69, CD80, CD86, CD90 or their ligands. The IL-17F signaling antagonists may also be combined with agents, such as methotrexate, cyclosporine, FK506, rapamycin, mycophenolate mofetil, leflunomide, NSAIDs, for example, ibuprofen, corticosteroids such as prednisolone, phosphodiesterase inhibitors, adenosine agonists, antithrombotic agents, complement inhibitors, adrenergic agents, agents which interfere with signaling by proinflammatory cytokines as described herein, IL-1b converting enzyme inhibitors (e.g., Vx740), anti-P7s, PSGL, TACE inhibitors, T-cell signaling inhibitors such as kinase inhibitors, metalloproteinase inhibitors, sulfasalazine, azathloprine, 6-mercaptopurines, angiotensin converting enzyme inhibitors, soluble cytokine receptors and derivatives thereof, as described herein, and anti-inflammatory cytokines (e.g. IL-4, IL-10, IL-13 and TGF).

Preferred examples of therapeutic agents for multiple sclerosis with which the IL-17F signaling antagonists can be combined include interferon-β, for example, IFNβ-1a and IFNβ-1b; copaxone, corticosteroids, IL-1 inhibitors, TNF inhibitors, antibodies to CD40 ligand and CD80, IL-12 antagonists.

Nonlimiting examples of agents for treating or preventing inflammatory bowel disease (e.g., Crohn's disease, ulcerative colitis) with which a IL-17F signaling antagonist (e.g., IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17R, or IL-17RC antibodies; and/or antagonistic small molecules, etc.) can be combined include the following: budenoside; epidermal growth factor; corticosteroids; cyclosporine; sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1 monoclonal antibodies; anti-IL-6 monoclonal antibodies; growth factors; elastase inhibitors; pyridinyl-imidazole compounds; TNF antagonists as described herein; IL-4, IL-10, IL-13 and/or TGFβ cytokines or agonists thereof (e.g., agonist antibodies); IL-11; glucuronide- or dextran-conjugated prodrugs of prednisolone, dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of platelet activating factor (PAF); ciprofloxacin; and lignocaine.

In one embodiment, an IL-17F signaling antagonist (e.g., IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17R, or IL-17RC antibodies; and/or antagonistic small molecules, etc.) can be used in combination with one or more antibodies directed at other targets involved in regulating immune responses, e.g., transplant rejection. Nonlimiting examples of agents for treating or preventing immune responses with which an IL-17F signaling antagonist of the invention can be combined include the following: antibodies against other cell surface molecules, including but not limited to CD25 (interleukin-2 receptor-a), CD11a (LFA-1), CD54 (ICAM-1), CD4, CD45, CD28/CTLA4 (CD80 (B7.1), e.g., CTLA4 Ig—abatacept (ORENCIA®)), ICOSL, ICOS and/or CD86 (B7.2). In yet another embodiment, an IL-17F signaling antagonist is used in combination with one or more general immunosuppressive agents, such as cyclosporin A or FK506.

In other embodiments, IL-17F signaling antagonists (e.g., IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17R, or IL-17RC antibodies; and/or antagonistic small molecules, etc.) are used as vaccine adjuvants against autoimmune disorders, inflammatory diseases, etc. The combination of adjuvants for treatment of these types of disorders are suitable for use in combination with a wide variety of antigens from targeted self-antigens, i.e., autoantigens, involved in autoimmunity, e.g., myelin basic protein; inflammatory self-antigens, e.g., amyloid peptide protein, or transplant antigens, e.g., alloantigens. The antigen may comprise peptides or polypeptides derived from proteins, as well as fragments of any of the following: saccharides, proteins, polynucleotides or oligonucleotides, autoantigens, amyloid peptide protein, transplant antigens, allergens, or other macromolecular components. In some instances, more than one antigen is included in the antigenic composition.

For example, desirable vaccines for moderating responses to allergens in a vertebrate host, which contain the adjuvant combinations of this invention, include those containing an allergen or fragment thereof. Examples of such allergens are described in U.S. Pat. No. 5,830,877 and published International Patent Application No. WO 99/51259, which are hereby incorporated by reference in their entireties, and include pollen, insect venoms, animal dander, fungal spores and drugs (such as penicillin). The vaccines interfere with the production of IgE antibodies, a known cause of allergic reactions. In another example, desirable vaccines for preventing or treating disease characterized by amyloid deposition in a vertebrate host, which contain the adjuvant combinations of this invention, include those containing portions of amyloid peptide protein (APP). This disease is referred to variously as Alzheimer's disease, amyloidosis or amyloidogenic disease. Thus, the vaccines of this invention include the adjuvant combinations of this invention plus Aβ peptide, as well as fragments of Aβ peptide and antibodies to Aβ peptide or fragments thereof.

Methods of: 1) downregulating antigen presenting cell function; and 2) combination therapy for managing immunosuppression are well known in the art (see, e.g., Xiao et al. (2003) BioDrugs 17:103-11; Kuwana (2002) Hum. Immunol. 63:1156-63; Lu et al. (2002) Transplantation 73:S19-S22; Rifle et al. (2002) Transplantation 73:S1-S2; Mancini et al. (2004) Crit. Care. Nurs. Q. 27:61-64).

Another aspect of the present invention accordingly relates to kits for carrying out the administration of IL-17F signaling antagonists (e.g., IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17R, or IL-17RC antibodies; and/or antagonistic small molecules, etc.) with other therapeutic compounds. In one embodiment, the kit comprises one or more binding agents formulated in a pharmaceutical carrier, and at least one agent, e.g., therapeutic agent, formulated as appropriate, in one or more separate pharmaceutical preparations.

The entire contents of all references, patents, and published patent applications cited throughout this application are hereby incorporated by reference herein.

### EXAMPLES

The following Examples provide illustrative embodiments of the invention and do not in any way limit the invention. One of ordinary skill in the art will recognize that numerous other embodiments are encompassed within the scope of the invention.

The Examples do not include detailed descriptions of conventional methods, such methods employed in the construction of vectors, the insertion of genes encoding the polypeptides into such vectors and plasmids, the introduction of such vectors and plasmids into host cells, and the expression of polypeptides from such vectors and plasmids in host cells. Such methods are well known to those of ordinary skill in the art.

### Example 1

### IL-17F-Mediated Inflammatory Responses and Implications in Inflammatory Disorders, e.g., Rheumatoid Arthritis, Inflammatory Respiratory Disorders, and Inflammatory Bowel Disease

### Example 1.1

### Human IL-17F Administration in Naïve Mice Induces Neutrophil Influx into the Peritoneum

The observation that IL-17F treatment results in increased proteoglycan breakdown and decreased proteoglycan synthesis by articular cartilage (Hymowitz et al. (2001) EMBO J. 20:5332-41) suggests a role for IL-17F signaling in the development of inflammatory diseases of joint tissue. Indeed, synovial fluid samples from patients with rheumatoid arthritis and osteoarthritis show degradation of proteoglycans including, e.g., aggrecan, keratin, and collagen (see, e.g., Witter et al. (1987) Arth. Rheum. 30:519-29 and Yagi et al. (2005) J. Orthop. Res. 23(5):1128-38), and in vitro arthritis models mimic this phenomenon by displaying matrix and proteoglycan degradation (Neidhart et al. (2000) Arth. Rheum. 43:1719-28).

Additionally, the increased expression of IL-17F observed in BAL samples isolated from patients suffering from asthma (Kawaguchi et al. (2002) J. Immunol. 167:4430-35) and colon samples isolated from patients suffering from inflammatory bowel diseases, e.g., ulcerative colitis or Crohn's disease (Gurney et al. (2003) GTCBIO Conference: Cytokines and Beyond) suggests an additional role for IL-17F signaling in inflammatory disorders of lung and bowel tissues. To investigate the role of IL-17F signaling in inflammatory responses, naïve mice were injected intraperitoneally with PBS (as a control) or 100 μg human IL-17F (SEQ ID NO:2). Hours after treatment, samples from peripheral blood (PB; 1, 2, 4, 6, 8, and 10 h) and peritoneal cavities (PEC; 2, 4, 6, 8, and 10 h) were taken, and the absolute neutrophil count (ANC) in each sample was determined. The data in Table 2 reflects the ability of IL-17F to increase neutrophil counts in blood and peritoneum. This effect may explain the neutrophilia seen in patients suffering from rheumatoid arthritis and chronic obstructive pulmonary diseases (COPD).

TABLE 2
Absolute neutrophil counts (ANC; mean ± SEM) in peripheral
blood (PB) and peritoneal cavity (PEC) samples isolated
from mice injected with PBS (n = 5) or IL-17F (n = 5).

| Hours post | PB ANC × 10³/uL | | PEC ANC × 10⁵/mL | |
|---|---|---|---|---|
| injection | PBS | IL-17F | PBS | IL-17F |
| 1 | 1.24 ± 0.59 | 1.97 ± 0.45 | — | — |
| 2 | 0.15 ± 0.10 | 1.07 ± 0.25* | 0.08 ± 0.05 | 2.28 ± 2.52 |
| 4 | 0.15 ± 0.08 | 0.75 ± 0.13* | 0.00 ± 0.01 | 0.30 ± 0.15* |
| 6 | 0.25 ± 0.13 | 0.93 ± 0.4* | 0.04 ± 0.06 | 2.03 ± 1.35* |
| 8 | 0.22 ± 0.06 | 0.31 ± 0.08 | 0.02 ± 0.02 | 1.20 ± 2.11 |
| 10 | 0.11 ± 0.09 | 0.48 ± 0.36 | 0.02 ± 0.03 | 2.06 ± 1.53* |

Asterisk denotes a p-value < 0.05 compared to control samples.

### Example 1.2

### IL-17F Signaling Plays a Role in Inflammatory Joint Disorders

To further assess the involvement of IL-17F signaling in inflammation, particularly inflammatory responses implicated in joint diseases (e.g., arthritis), the ability of IL-17F to activate a primary factor involved in the transcription of inflammatory cytokines, i.e., NF-κB, in primary chondrocytes was determined. Primary human or porcine chondrocytes were infected (100 MOI) with adenovirus expressing an NF-κB reporter gene system, which detects activation of endogenous NF-κB (i.e., translocation of NF-κB from the cytoplasm to the nucleus) by measuring the expression of a luciferase gene that is controlled by an NF-κB-responsive promoter (BD Mercury Pathway Profiling systems, BD Biosciences, Palo Alto, Calif.). After 48-72 hours, infected chondrocytes were cultured with varying concentrations of IL-17A (SEQ ID NO:4) or IL-17F. After four hours of incubation with IL-17A or IL-17F, cells were lysed in 25 μl lysis buffer (Promega, Madison, Wis.) for 20 min at RT, and activation of NF-κB was measured using an automated luminometer. The data show that IL-17F activated NF-κB in primary human chondrocytes (FIG. 1A) and primary porcine chondrocytes (FIG. 1B) in a dose-dependent manner. Finally, the amount of IL-17F required to activate NF-κB to levels above background was similar to the amount of IL-17A required to activate NF-κB to levels above background (FIG. 1A).

The ability of IL-17F to activate the cytokine transcription factor, NF-κB, in primary chondrocytes suggests that the role IL-17F plays in inflammation of joint tissue, e.g., during the development of arthritis, involves the induction of inflammatory cytokines. To test the effect of IL-17F on inflammatory cytokine production by joint tissues, human fibroblast-like synoviocytes isolated from two patients diagnosed with rheumatoid arthritis (RA) were plated to semi-confluence in 24 well plates and cultured in the absence or presence of 150 ng/ml IL-17F. Supernatants were collected at 48 h and cytokine production assessed using a multiplex cytokine system (Pierce-Bio, Rockford, Ill.). As shown in FIG. 2, IL-17F induced the production of inflammatory cytokines such as IL-6 and IL-8, and chemokines, such as MCP-1 and GRO-α, by human fibroblast-like synoviocytes isolated from RA patients.

The ability of IL-17F to activate NF-κB in primary chondrocytes and induce production of inflammatory cytokines and chemokines, particularly chemokines involved in neutrophil recruitment, by human fibroblast-like synoviocytes from RA patients supports a role for IL-17F in mediating inflammatory responses by joint tissue. These data, taken together with data demonstrating increased IL-17F expression in the paws of mice suffering from collagen induced arthritis compared to control animals (data not shown) and the presence of neutrophils within degenerated articular cartilage and joint space (data not shown), suggests that IL-17F mediates inflammatory joint diseases (e.g., rheumatoid arthritis) by inducing cytokine and chemokine production, which subsequently recruits, to the site of inflammation, immune cells (e.g., neutrophils) that cause damage to surrounding tissues.

### Example 1.3

### Primary Mouse Lung Fibroblasts Respond to IL-17F by Upregulating Production of Inflammatory Chemokines and Cytokines

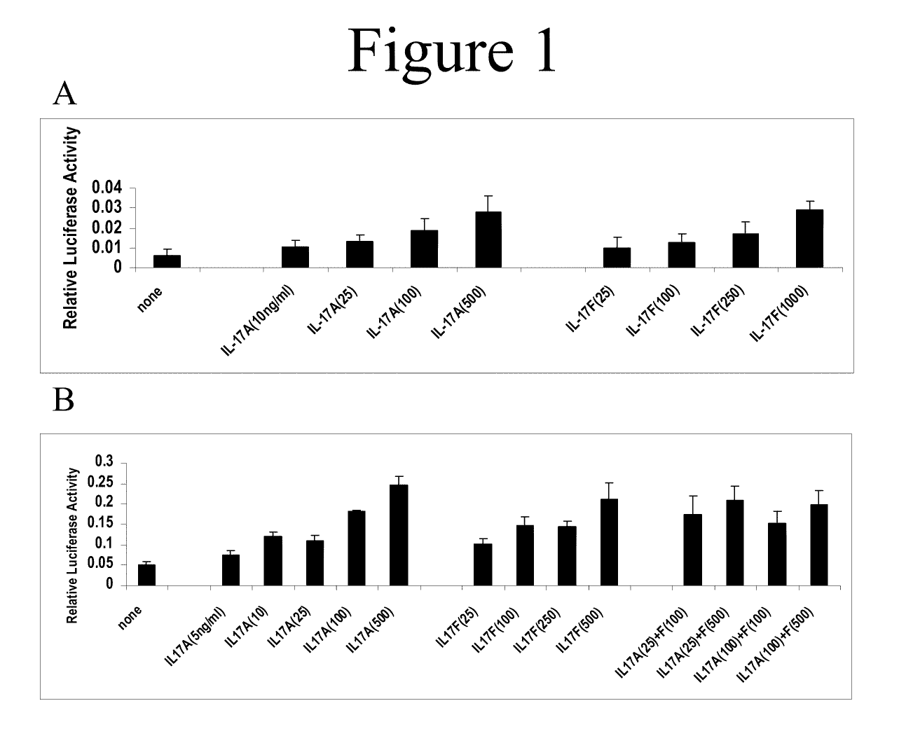

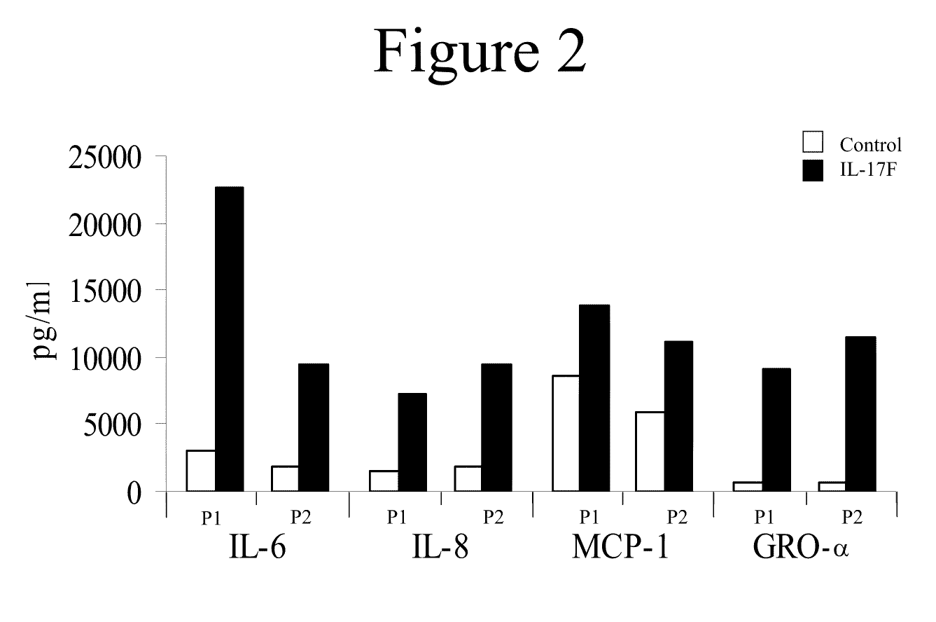

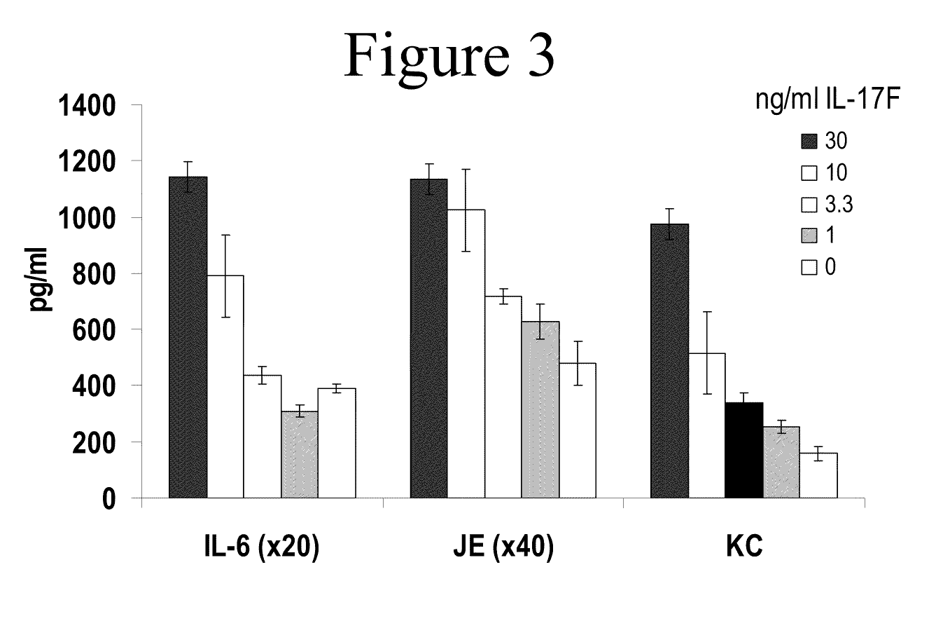

To test the role of IL-17F in the development of inflammatory respiratory diseases, murine lung fibroblast (MFL) cells were grown to semi-confluence on 24 well plates and treated with IL-17F (50 ng/ml). Supernatants were collected at 48 h, and cytokine production was assessed using a multiplex cytokine system (Pierce-Bio, Rockford, Ill.). FIG. 3 shows that IL-17F induced the MFL production of inflammatory cytokines, such as IL-6, and chemokines, such as JE (CCL2) and KC. These results suggest IL-17F contributes to inflammatory responses implicated in inflammatory disorders of the lung (e.g., COPD, asthma, allergy, cystic fibrosis) by inducing the production of inflammatory cytokines and leukocyte chemoattractants.

### Example 2

### Characterization of IL-17F Receptors

### Example 2.1

### IL-17F Binds to IL-17R and IL-17RC

As IL-17F shares the greatest homology with IL-17A within the IL-17 family, and as it has been suggested that IL-17A and IL-17F signal via the IL-17 receptor, the ability of IL-17F to bind to the receptor for IL-17A (i.e., IL-17R; SEQ ID NO:6) was determined. The ability of IL-17F to bind to IL-17RC, an IL-17 receptor whose ligand to date has not been identified, was also tested.

ELISA plates were incubated with 1.5 μg/ml human IL-17R-Ig (SEQ ID NO:34) or 1.5 μg/ml human IL-17RC-Ig (SEQ ID NO:35) overnight. Plates were washed with PBS/1% BSA and incubated with serial dilutions of biotin-conjugated IL-17A or biotin-conjugated IL-17F for 2 h at room temperature (RT). After washing, saturating concentrations of avidin-horseradish peroxidase (HRP) were added, and plates were incubated for an additional 1 h at RT. Unbound avidin-HRP was washed using PBS/1% BSA, and the ELISA was developed using TBM. Bound IL-17A or IL-17F was detected by measuring the absorbance at 405 nm.

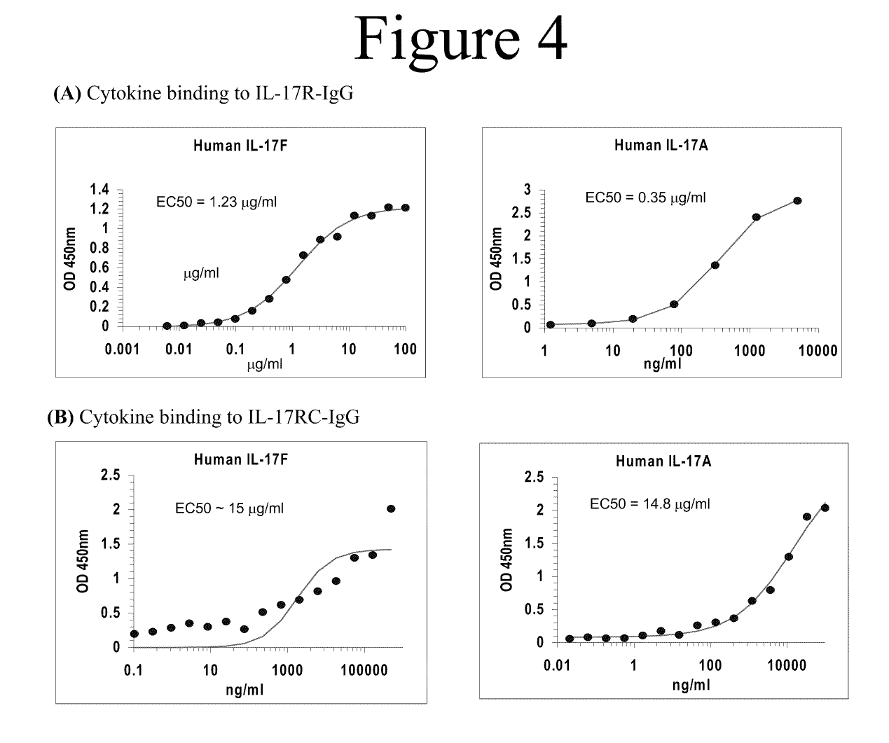

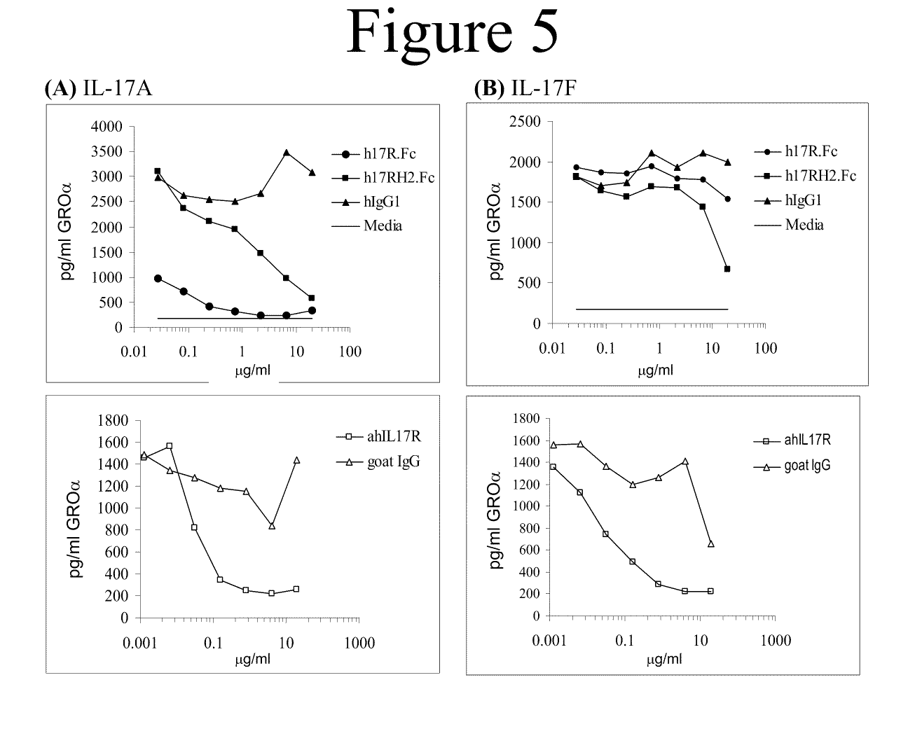

FIGS. 4A and 4B demonstrate binding of IL-17F to both IL-17R and IL-17RC, respectively, with IL-17F having a greater affinity for IL-17R compared to its affinity for IL-17RC (EC₅₀ value for IL-17R:IL-17F=1.23 μg/ml; EC₅₀ value for IL-17RC:IL-17F=15 μg/ml). However, although IL-17F bound to IL-17R, its affinity for the receptor was lower than that of the affinity of IL-17A for the same receptor (EC₅₀ values for IL-17R: IL-17F=1.23 μg/ml, IL-17R:IL-17A=0.35 μg/ml; FIG. 4A).

### Example 2.2

### Anti-IL-17R Antibody and IL-17RC-Ig Fusion Protein, but not IL-17R-Ig Fusion Protein, Block IL-17F Activity

To further characterize IL-17F receptor binding, human fibroblast cells (10⁴ cells/well) were stimulated with 0.5 ng/ml IL-17A or 20 ng/ml IL-17F in the presence of increasing concentrations of an IL-17R-Ig fusion protein, an IL-17RC-Ig fusion protein or an anti-IL-17R antibody. After 24 h, the GRO-α concentrations of collected supernatants were determined using a commercially available ELISA (R&D, Minneapolis, Minn.). Concentrations of GRO-α were determined based on a standard curve.

FIG. 5 demonstrates increased GRO-α production by human fibroblast cells when incubated with IL-17A (A) or IL-17F (B), and further corroborates the findings of Example 1.2 (demonstrating increased inflammatory cytokine production by human fibroblast-like synoviocytes cultured with IL-17F). FIG. 5A additionally shows that all three receptor antagonists, i.e., IL-17R-Ig (h17R.Fc), IL-17RC-Ig (h17RH2.Fc) and anti-IL-17R antibody (ahIL17R), blocked the ability of IL-17A to induce GRO-α. These data suggest that IL-17A binds to and requires both IL-17R and IL-17RC receptors for IL-17A signaling. In contrast, only the anti-IL-17R antibody and the IL-17RC-Ig fusion protein, and not the IL-17R-Ig fusion protein, notably blocked IL-17F activity (FIG. 5B). The data presented in FIG. 5B suggest that IL-17F binds to IL-17R (see FIG. 4), but does not require this receptor for IL-17F-mediated signaling. Altogether these data suggest IL-17A and IL-17F may use different receptors to mediate their activity on human fibroblast cells.

### Example 3

### Generation and Characterization of Anti-IL-17F Antibodies

### Example 3.1

### Generation of Anti-IL-17F Antibodies

A group of five mice (Jackson Labs, Maine) were injected with 2 μg of cDNA encoding human IL-17F. Purified plasmid cDNA was precipitated onto gold beads to a concentration of 1 μg cDNA/0.5 mg gold. The gold beads and precipitated cDNA were delivered, monthly in two nonoverlapping shots, intradermally in the abdomen of 11-week old female Balb/c mice using the Helios charged gene. These animals were immunized every four weeks and spleens removed at end of this period. Reimmunizations were performed using purified IL-17F protein in addition to IL-17F cDNA. Spleens were processed to obtain a lymphocyte suspension and the resulting suspension was fused with the myeloma cell line 653/P3 using 50% (w/v) polyethylene glycol 1500 by an established procedure (Oi and Herzenberg (1980) in Selected Methods in Cellular Immunology, Mishel and Schigi, eds. W. J. Freeman Co., San Francisco, Calif., p. 351). The fused cells were plated in 96-well microtiter plates at a density of 2×10⁵ cells/well, and after 24 hr were subjected to HAT selection. Hybridoma cells secreting putative anti-IL-17F antibodies were identified by solid and solution phase ELISA. Wells containing hybridoma positive for the above assays were expanded, cloned by limiting dilution and cryopreserved. Isotypes of antibodies were determined using solid phase ELISA. Purified human IL-17F-Ig was used to coat 96-well microtiter plates and detected by different isotype-specific biotin-conjugated goat anti-mouse IgG (Zymed, South San Francisco, Calif.). Streptavidin conjugated with horseradish peroxidase (HRP) was added and specifically bound enzyme measured using a colorimetric substrate.

### Example 3.2

### Some Anti-IL-17F Antibodies Inhibit IL-17F Binding to IL-17R

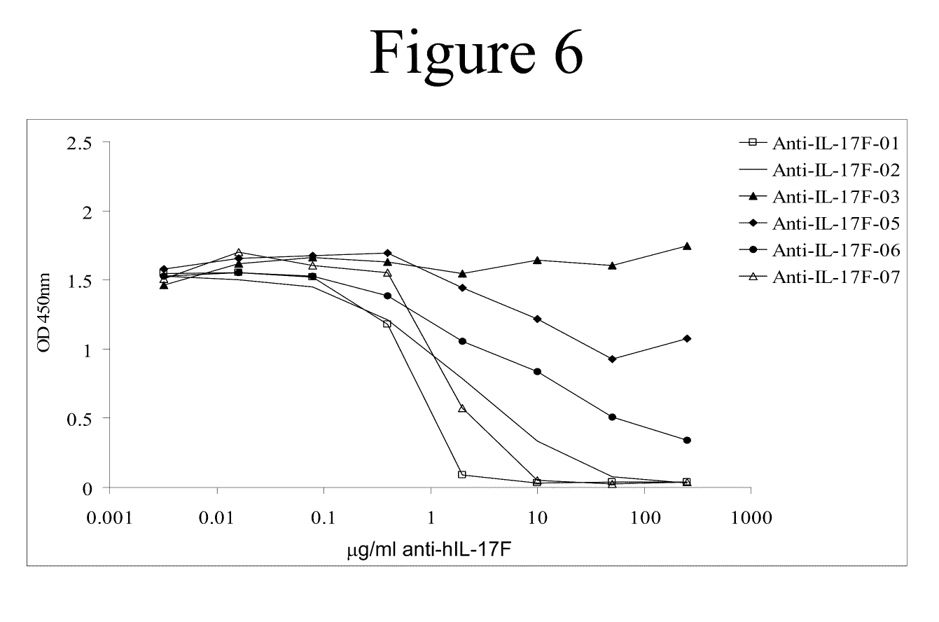

To assess the ability of the anti-IL-17F antibodies to block binding of IL-17F to IL-17R, inhibition assays were performed by modifying the ELISA described in Example 2.1. Briefly, serial dilutions of anti-IL-17F antibodies were preincubated with 7 μg/ml IL-17F for 1 h at RT. Each cytokine:antibody mixture was then added to separate wells of an ELISA plate previously coated with 100 μl/well of 1.5 μg/ml IL-17R-Ig. The mixture was incubated in the wells for 1 h at RT. After washing the plate with PBS/1% BSA, saturating concentrations of avidin-HRP were added, and the plate was incubated for an additional 1 h at RT. Unbound avidin-HRP was washed away using PBS/1% BSA. The assay was developed using TMB. FIG. 6 demonstrates that five out of six antibodies tested, i.e., anti-IL-17F-01, anti-IL-17F-02, anti-IL-17F-06, anti-IL-17F-07, and (albeit to a lesser degree) anti-IL-17F-05 were able to block binding of IL-17F to IL-17R. In contrast, anti-IL-17F-03 antibody did not inhibit IL-17F binding to IL-17R (FIG. 6).

### Example 3.3

### Some Anti-IL-17F Antibodies Inhibit IL-17F Binding to IL-17RC

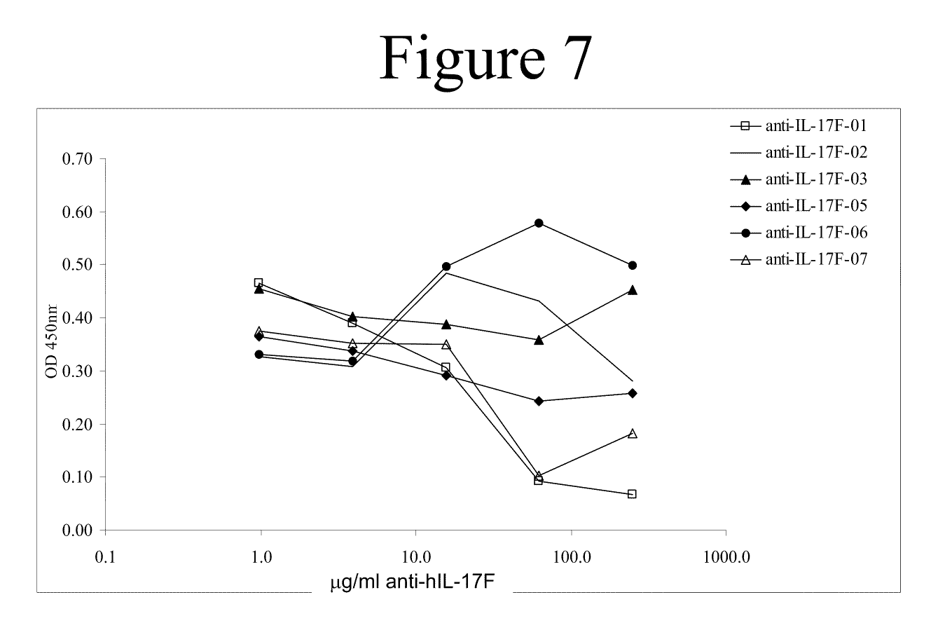

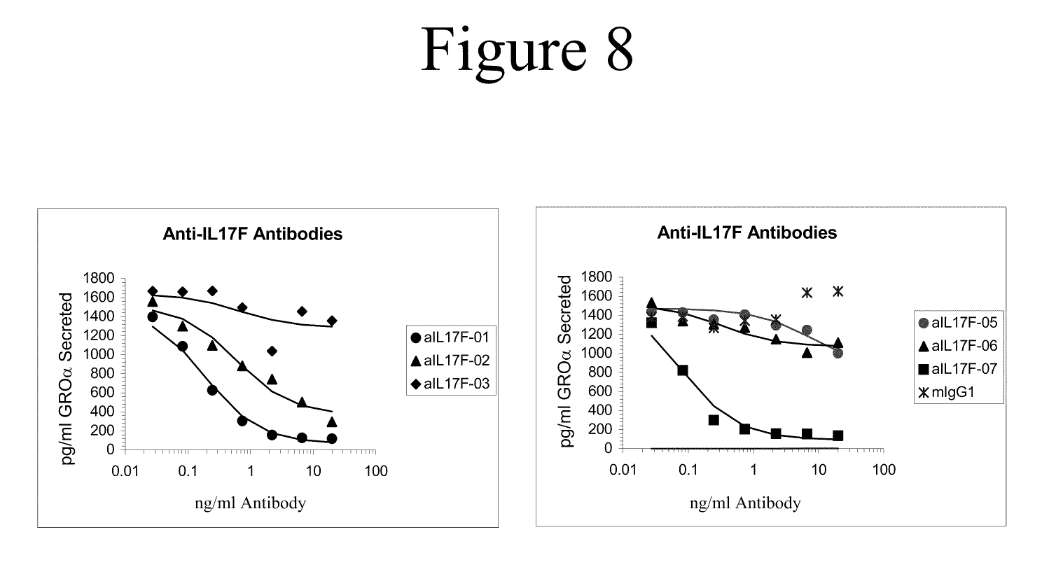

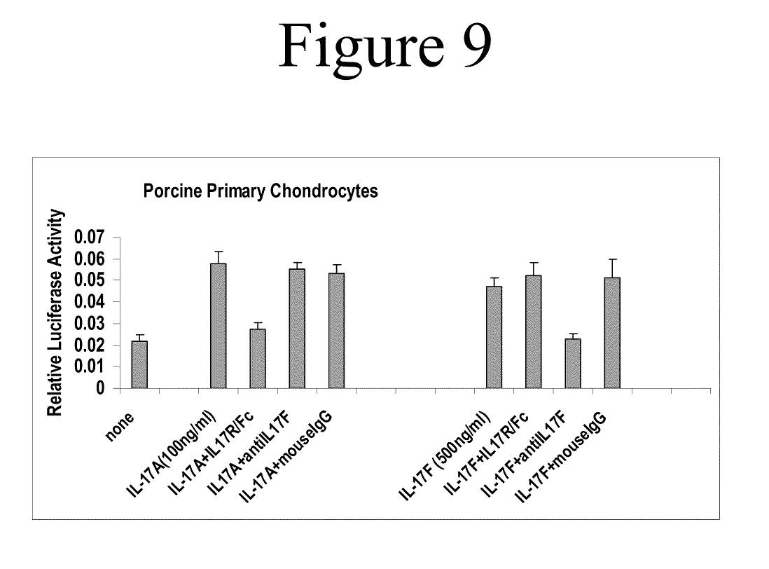

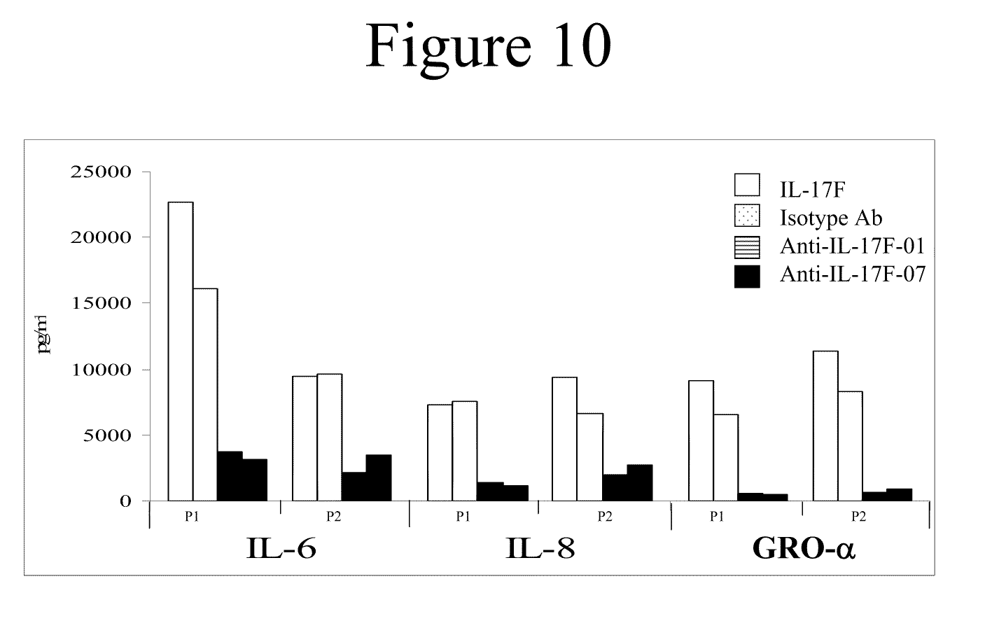

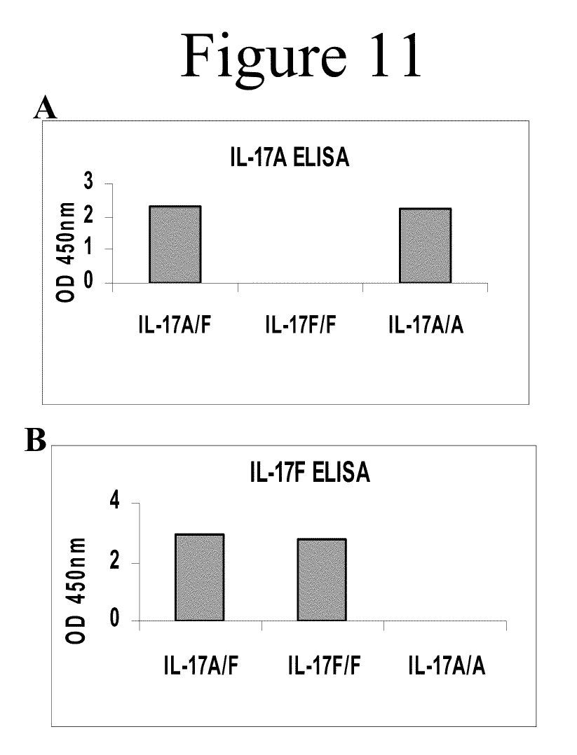

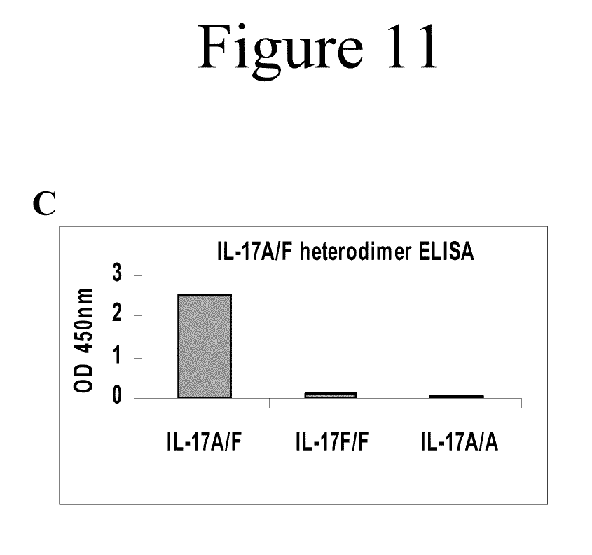

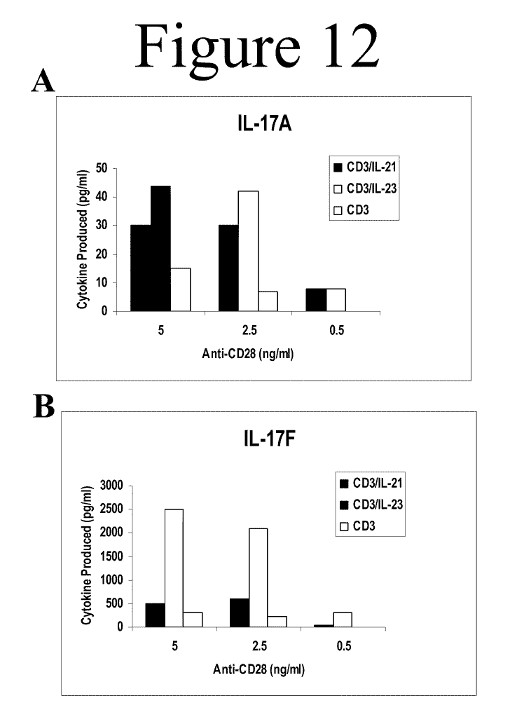

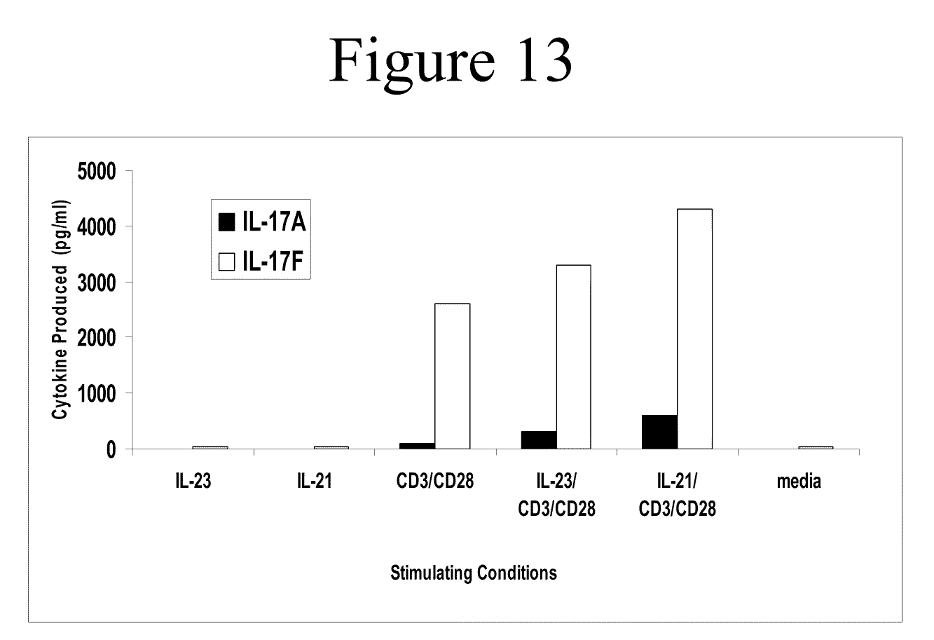

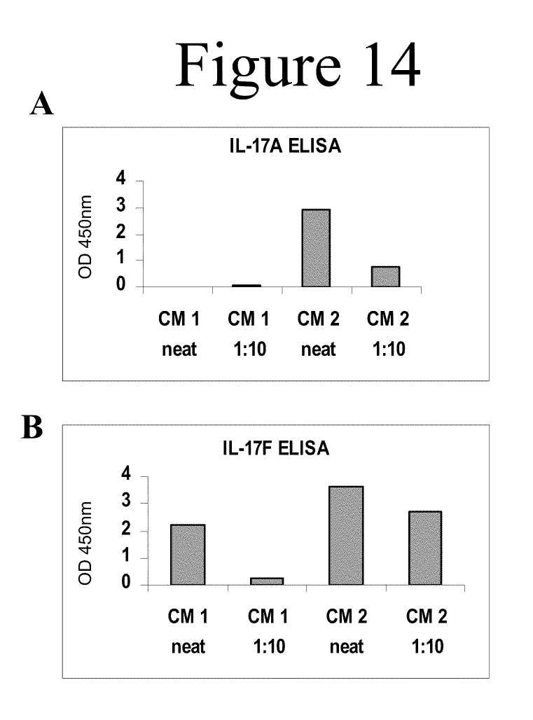

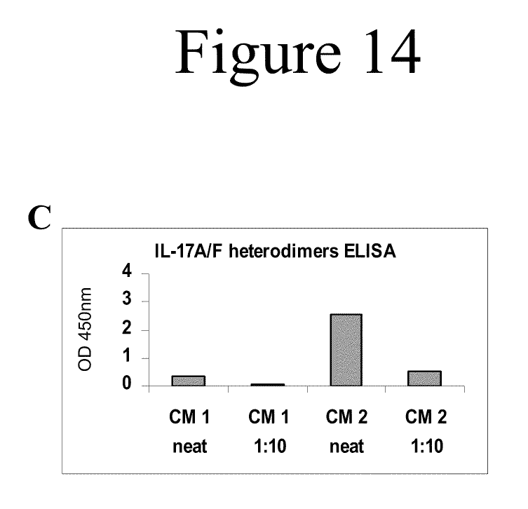

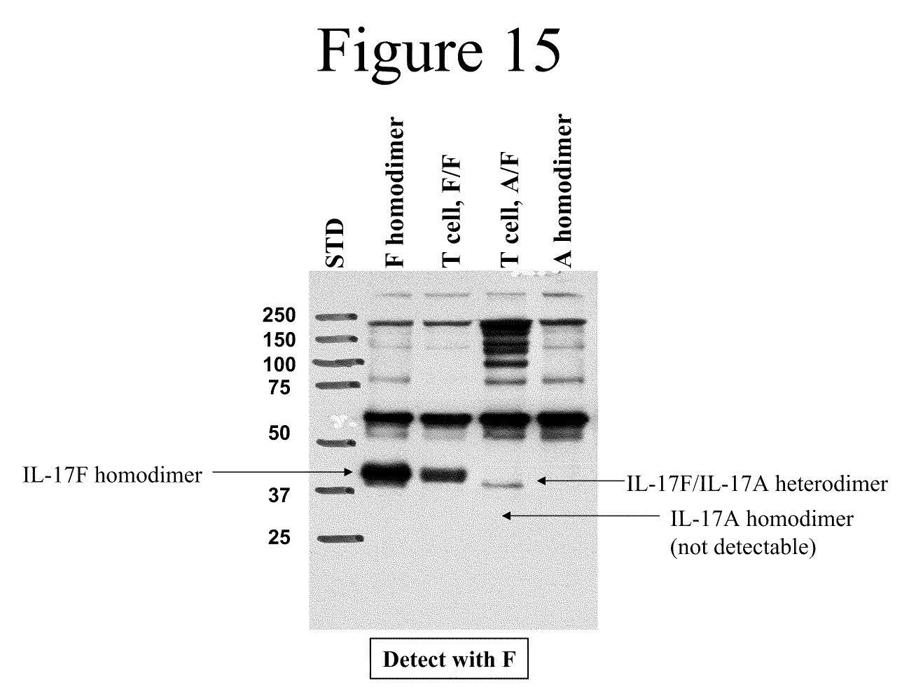

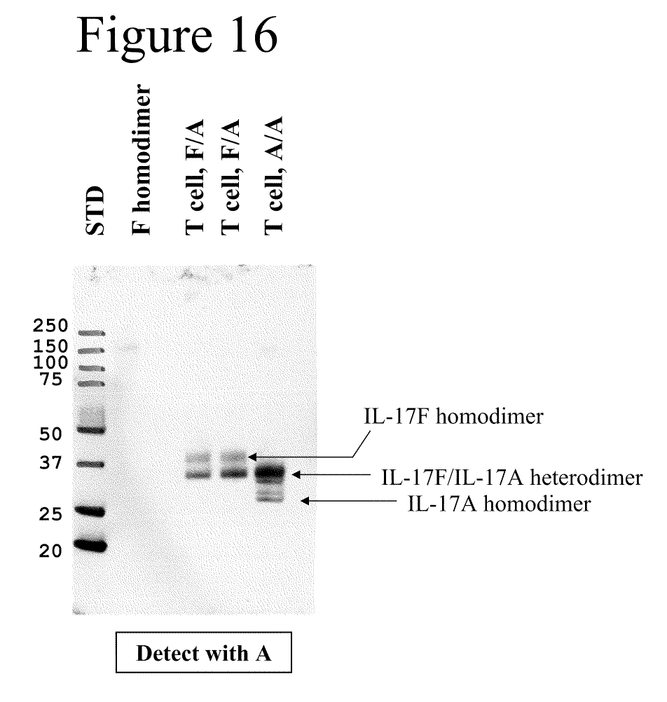

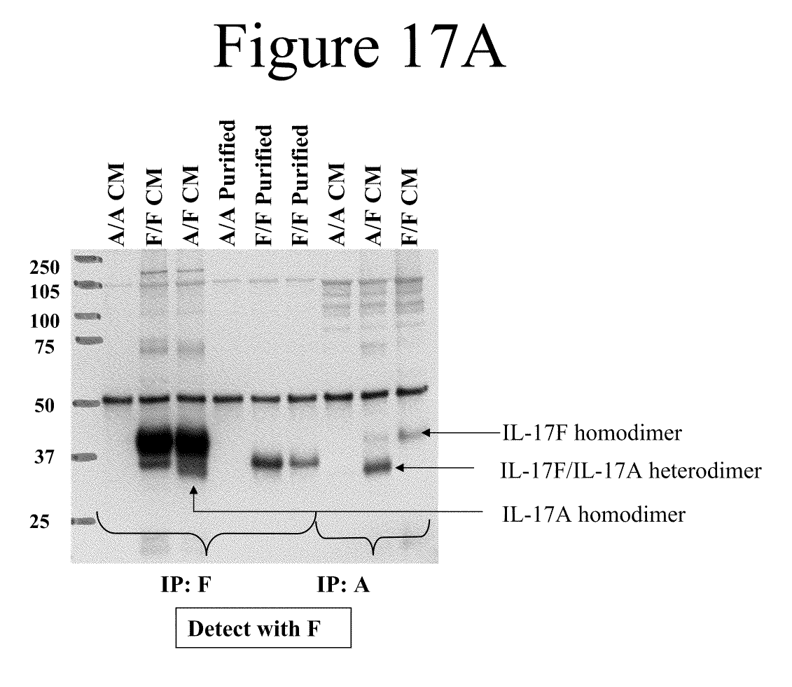

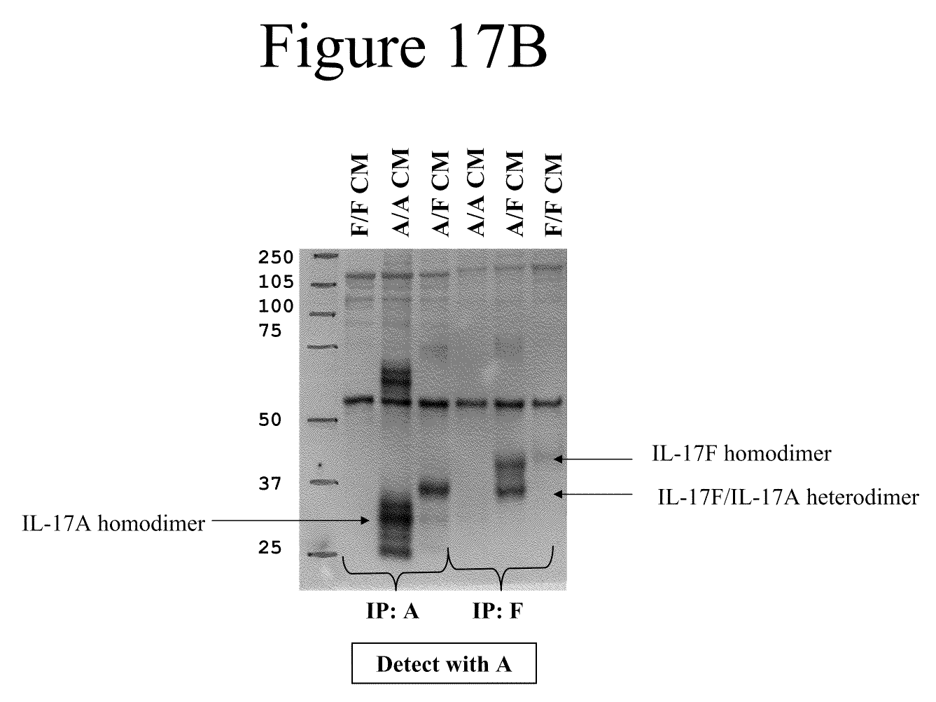

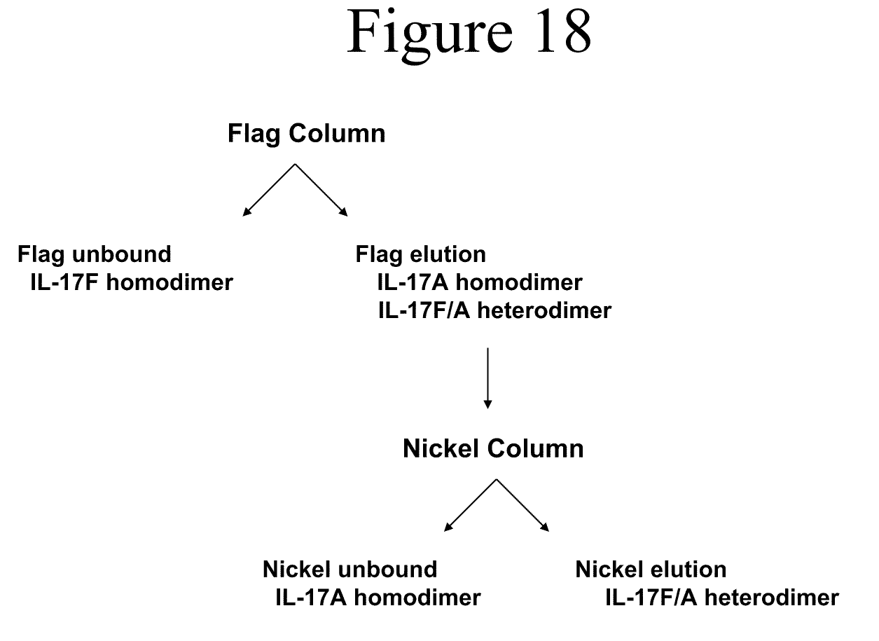

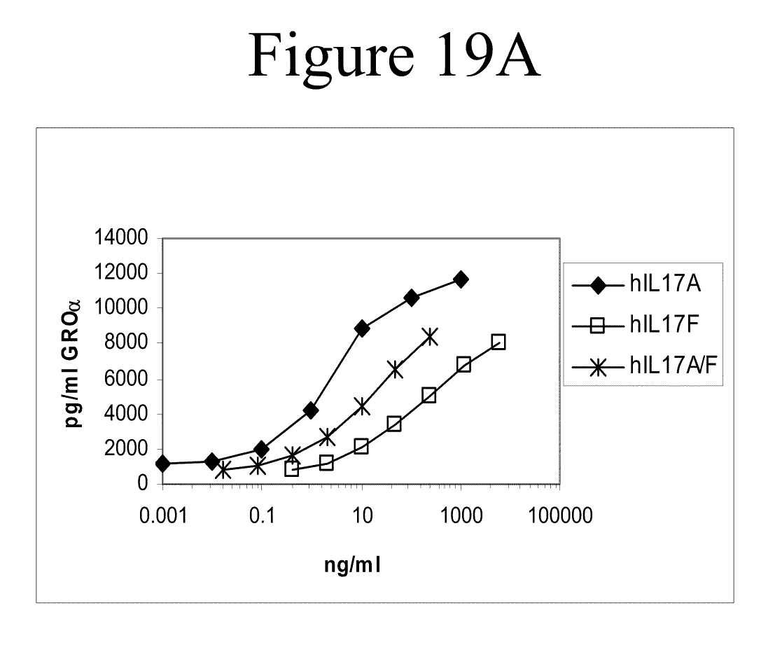

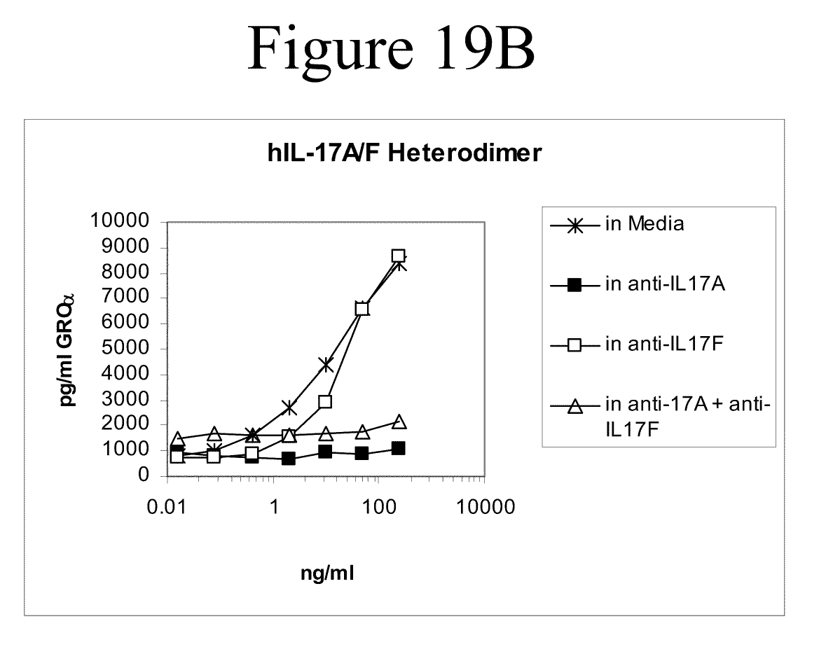

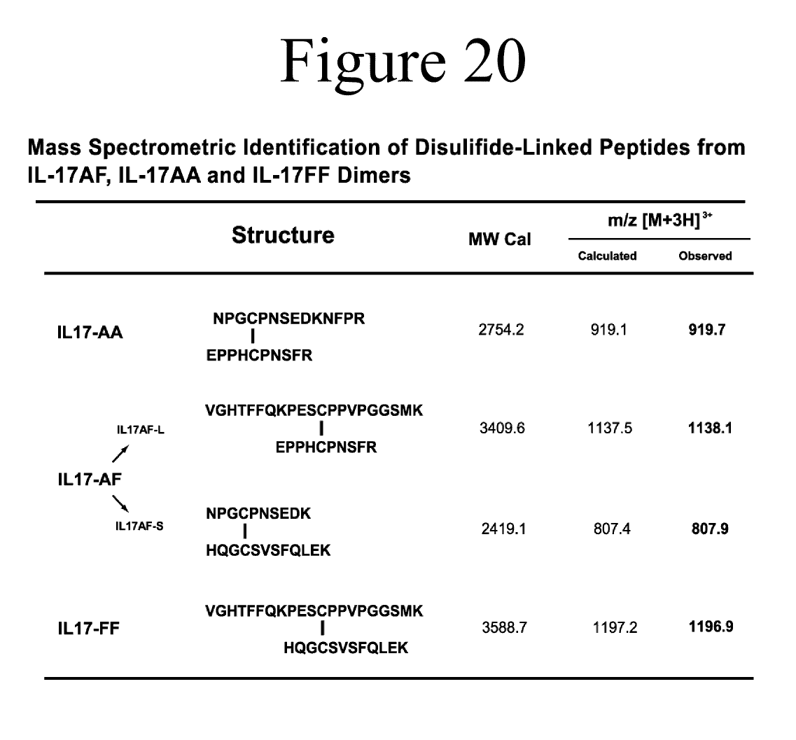

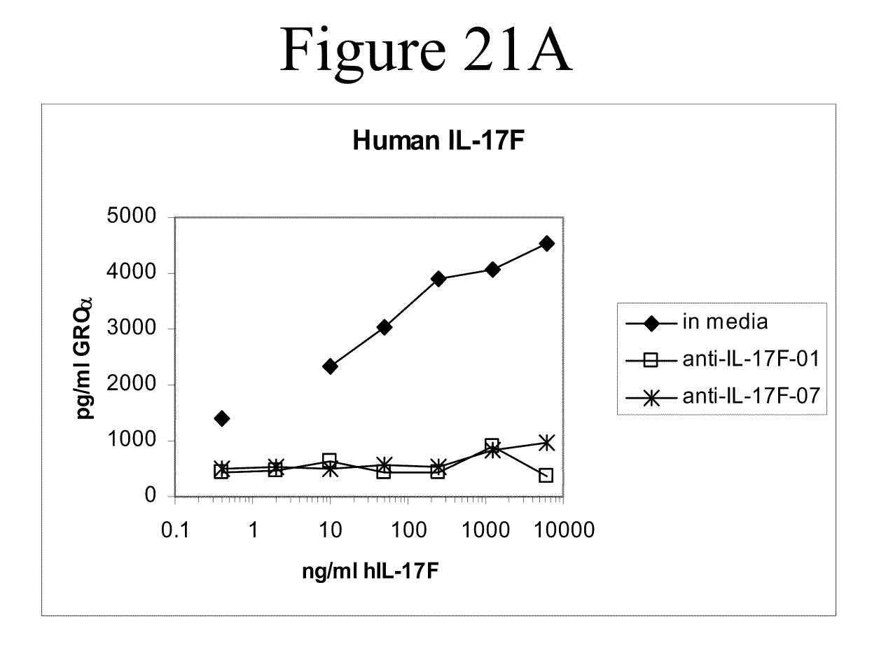

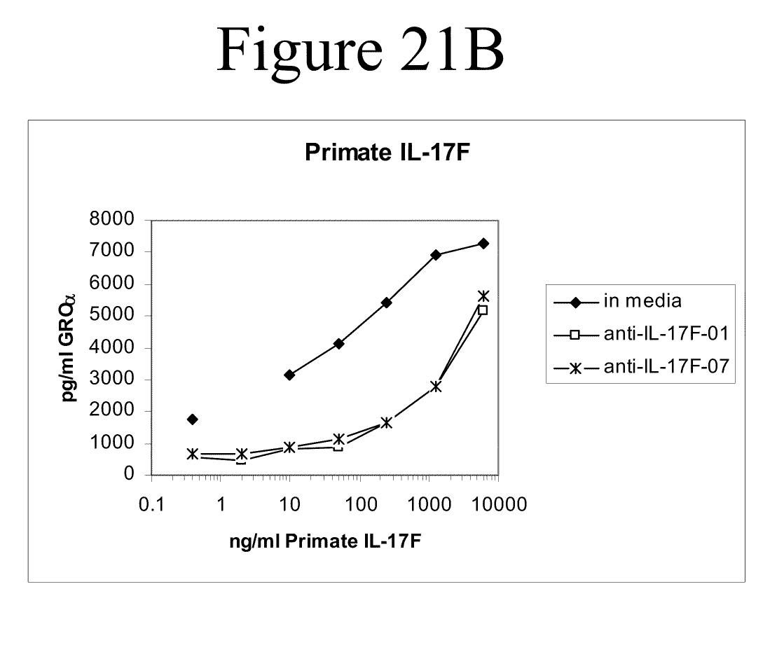

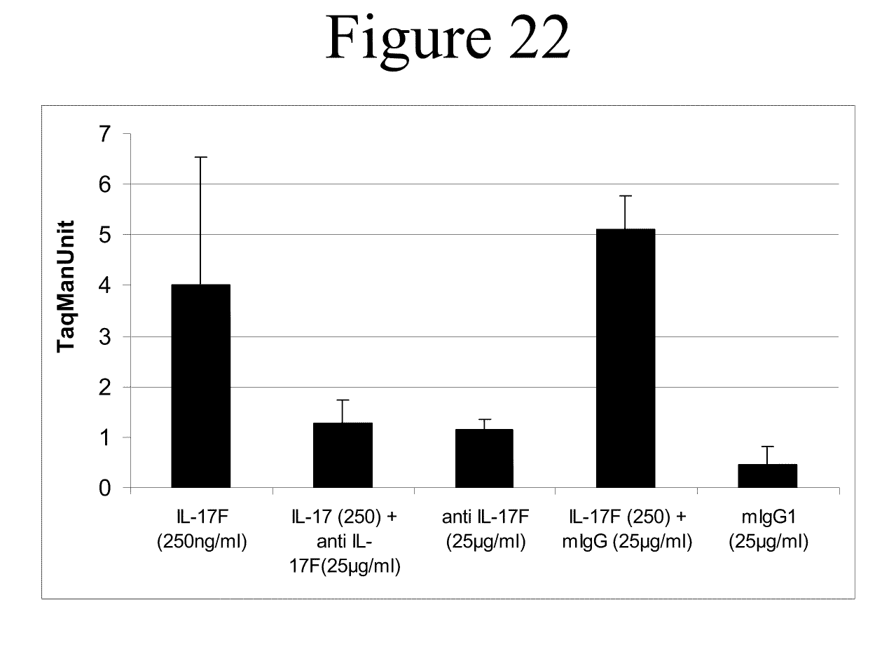

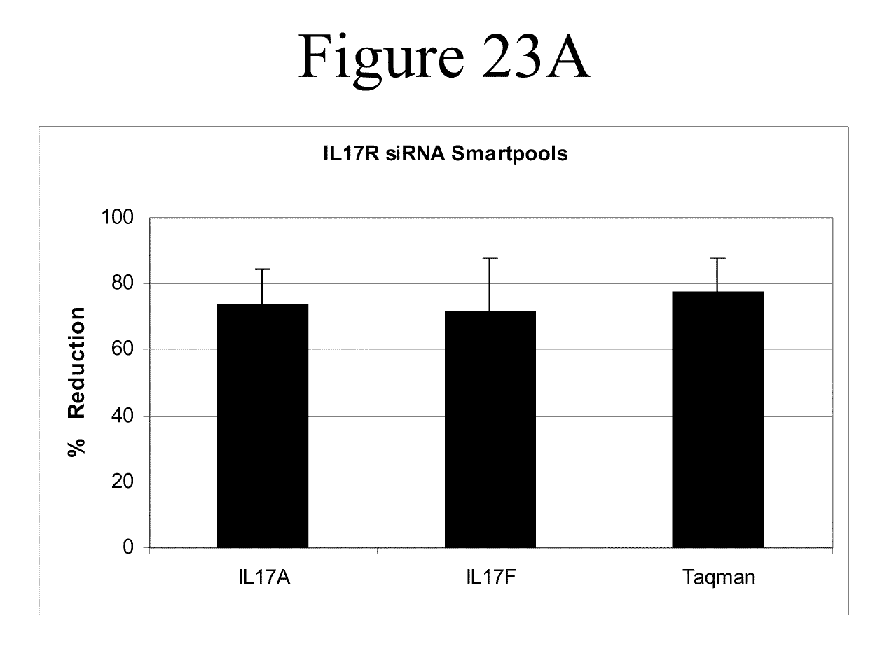

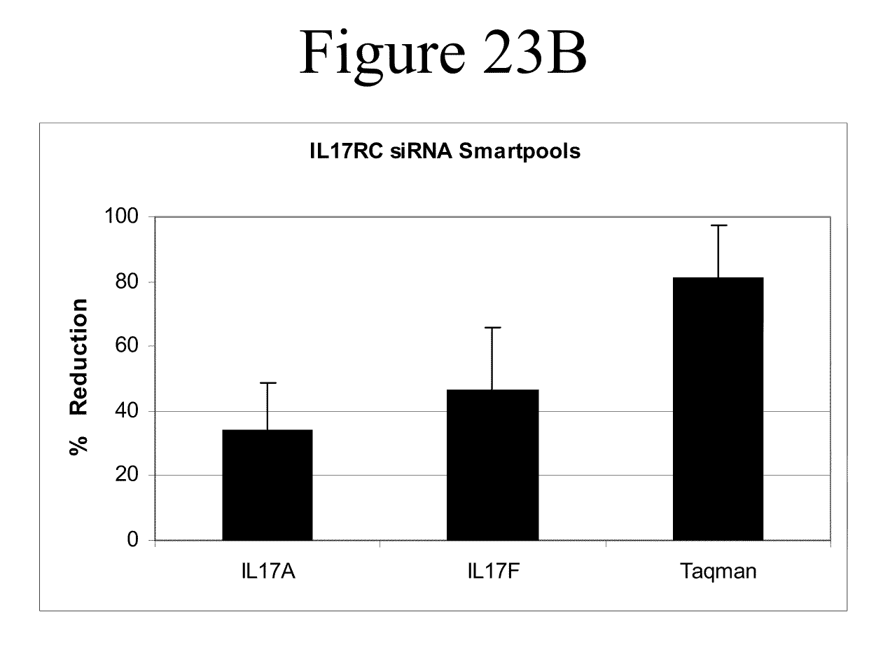

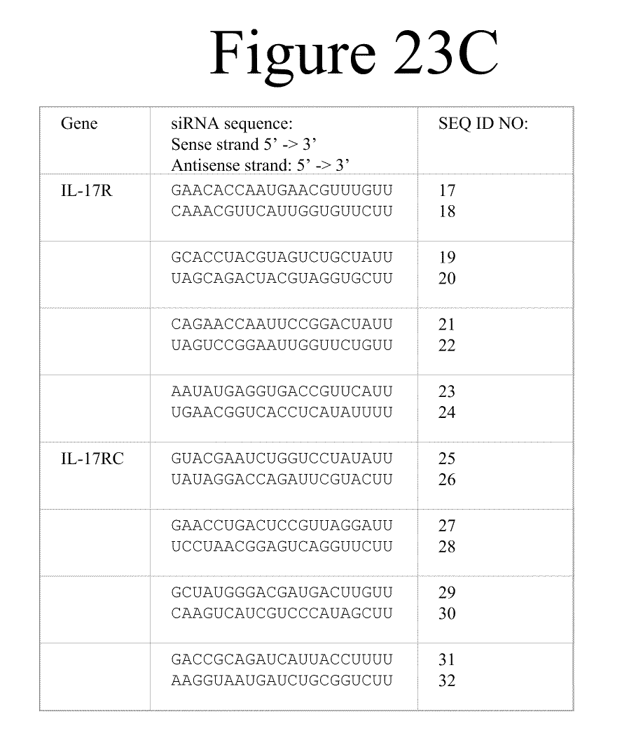

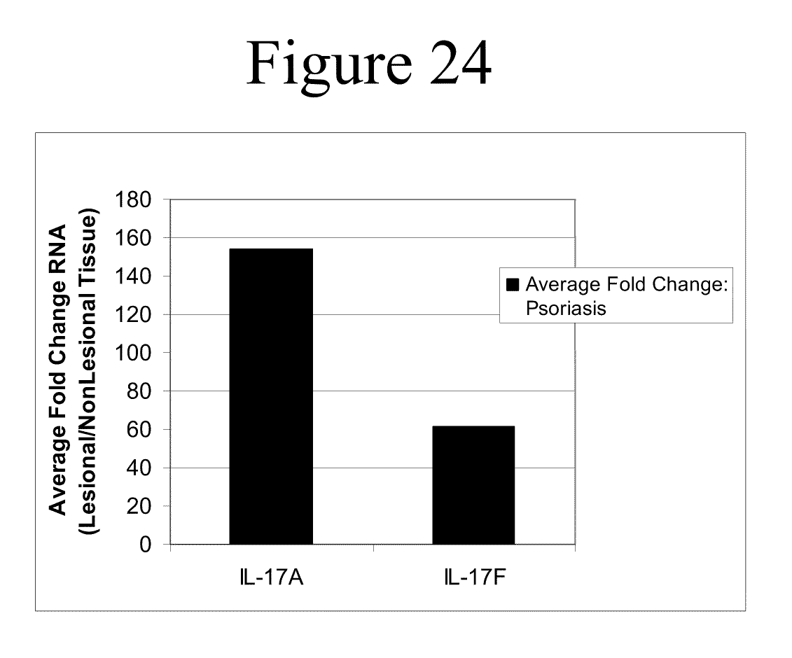

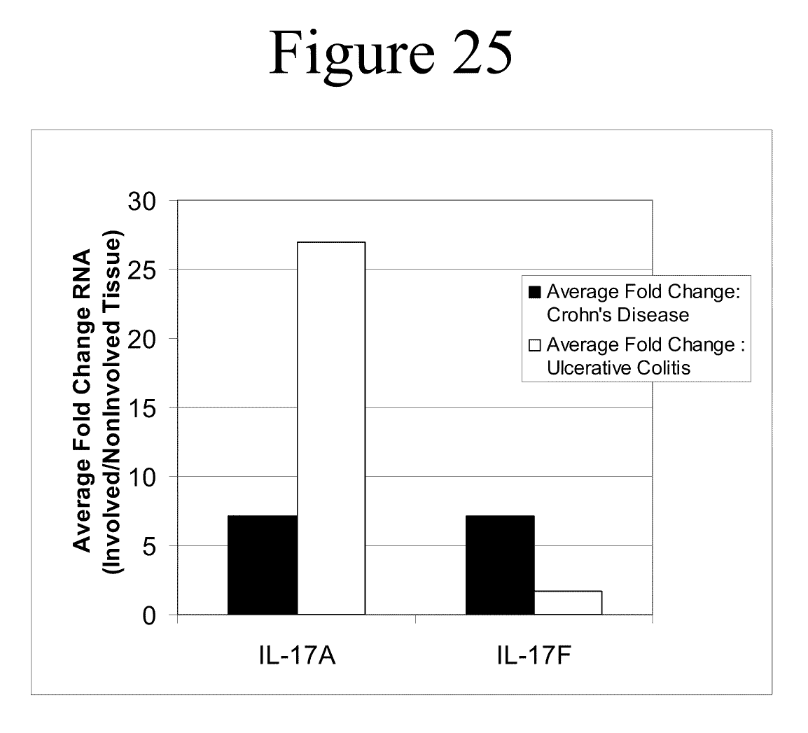

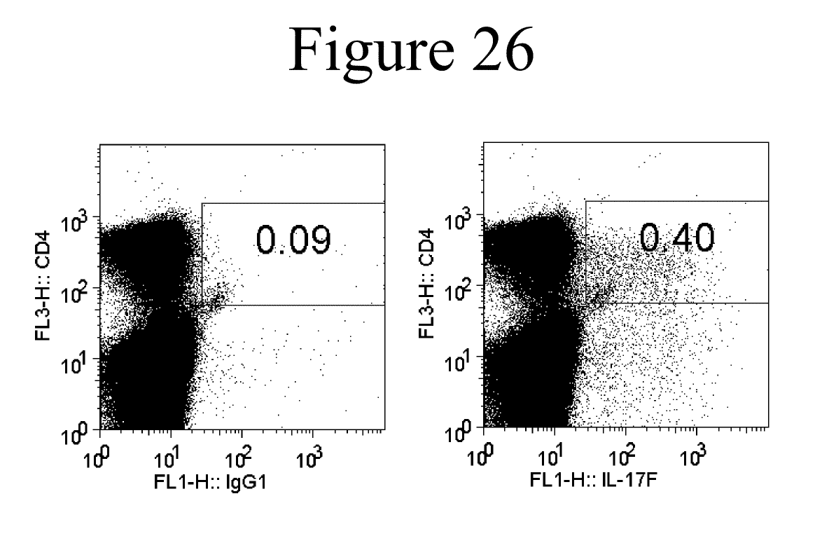

To assess the ability of anti-IL-17F antibodies to block binding of IL-17F to IL-17RC, inhibition assays using the modified ELISA, as described above, were performed using plates previously coated with 1.5 μg/ml IL-17RC-Ig and IL-17F at a concentration of 20 μg/ml. FIG. 7 demonstrates that two out of 6 antibodies tested (anti-IL-17F-01 and anti-IL-17F-07) were able to inhibit binding of IL-17F to IL-17RC. In contrast anti-IL-17F-02, anti-IL-17F-03, anti-IL-17F-05 and anti-IL-17F-06 antibodies did not inhibit IL-17F binding to IL-17RC (FIG. 7). Taking FIGS. 6 and 7 together, the data not only suggest that anti-IL-17F antibodies bind to distinct sites on IL-17F, but also that anti-IL-17F-02, and anti-IL-17F-06 antibodies bind to and/or inhibit binding to a site on IL-17F unique for the IL-17F:IL-17R interaction while anti-IL-17F-01 and anti-IL-17F-07 antibodies bind to and/or inhibit binding to a site on IL-17F shared between IL-17R and IL-17RC. Consequently, these six antibodies may be used to define distinct sites, i.e., epitopes, on IL-17F.

### Example 4

### Anti-IL-17F Antibodies Inhibit IL-17F Bioactivities

### Example 4.1

### Anti-IL-17F Antibodies Inhibit IL-17F-Mediated Cytokine Production

To determine whether any of the anti-IL-17F antibodies described in Example 3 inhibit IL-17F bioactivity, human fibroblast cells (10⁴ cells/well) were stimulated with human 20 ng/ml IL-17F in the presence of increasing concentrations of a control antibody (mIgG1) or antibodies to IL-17F, i.e., anti-IL-17F-01, anti-IL-17F-02, anti-IL-17F-03, anti-IL-17F-05, anti-IL-17F-06, or anti-IL-17F-07. After 24 h, supernatants were collected and GRO-α concentrations determined using commercially available ELISA. Concentrations of GRO-α produced were determined based on a standard curve.

FIG. 8 demonstrates that anti-IL-17F-01, anti-IL-17F-02, and anti-IL-17F-07 antibodies inhibited human IL-17F-mediated GRO-α production by human fibroblast cells. The results, presented in FIGS. 6, 7, and 8, suggest a model whereby IL-17F signaling is initiated by IL-17F binding to IL-17R, which results in the subsequent recruitment and required signaling through IL-17RC.

### Example 4.2

### Potential Uses of Anti-IL-17F Antibodies in the Treatment of Inflammatory Disorders

To determine the potential of anti-IL-17F antibodies, in particular anti-IL-17F-07 antibody, as a therapeutic in the treatment of disorders related to IL-17F signaling (e.g., autoimmune diseases, respiratory diseases, inflammatory bowel diseases, etc.), porcine primary chondrocytes infected with a NF-κB reporter vector were incubated for 48 h with 100 ng/ml IL-17A or 500 ng/ml IL-17F preincubated for 1 h at RT in the absence or presence of one of the following: 20 μg/ml IL-17R-Ig fusion protein (IL17R/Fc), 10 μg/ml anti-IL-17F-07 antibody (antiIL17F), or 10 μg/ml control antibody (mouseIgG). FIG. 9 demonstrates that while incubation of the IL-17R-Ig fusion protein inhibited IL-17A-mediated activation of NF-κB, it had no effect on IL-17F-mediated activation of NF-κB. In contrast, anti-IL-17F-07 antibody was able to inhibit IL-17F-mediated activation of NF-κB, but had no effect on IL-17A-mediated activation of NF-κB (FIG. 9).

To assess whether inhibited NF-κB activation in the presence of anti-IL-17F antibodies correlated with inhibited cytokine production, human fibroblast-like synoviocytes incubated with IL-17F, as described in Example 1.2, were incubated with an isotype control antibody, anti-IL-17F-01 antibody, or anti-IL-17F-07 antibody. Concentrations of IL-6, IL-8, or GRO-α were assessed as described in Example 1.2. FIG. 10 demonstrates that the ability of anti-IL-17F antibodies to inhibit IL-17F activation of NF-κB correlated with a decreased production of IL-6, IL-8, and GRO-α by primary fibroblast-like synoviocytes isolated from two patients with rheumatoid arthritis. These data suggest that antagonists of IL-17F signaling, including, but not limited to, inhibitory antibodies directed toward IL-17F, may be used to reduce inflammatory responses. In particular, inhibitors of IL-17F may be used in the treatment of inflammatory responses associated with disorders related to IL-17F signaling, i.e., IL-17F-associated disorders.

### Example 5

### Antibodies Directed Toward IL-17F and IL-17A May be Used to Detect and Purify Recombinant and Natural IL-17F Homodimers, IL-17A Homodimers, and IL-17A/IL-17F Heterodimers

### Example 5.1

### Detection of Recombinant IL-17A/IL-17A, IL-17F/IL-17F and IL-17A/IL-17F by ELISA

cDNAs encoding for human IL-17A, human IL-17F or both human IL-17A and human IL-17F were used to modify 293 cells. Expression of these cDNAs resulted in the production of IL-17A/IL-17A, IL-17F/IL-17F or IL-17A/IL-17F dimers. The conditioned media derived from these cells, i.e., the recombinant cytokines, and either commercially available antibodies or antibodies as described above, were used to develop ELISA formats for the detection of IL-17A protein, IL-17F protein, or IL-17A/IL-17F heterodimers. For the detection of IL-17A protein, i.e., as an IL-17A homodimer or an IL-17A/IL-17F heterodimer, anti-IL-17A antibody was used as a capture antibody and biotin labeled anti-IL-17A antibody was used as the detection antibody (both antibodies are available from R&D Systems, Minneapolis, Minn.). For the detection of IL-17F protein, i.e., as an IL-17F homodimer or an IL-17A/IL-17F heterodimer, anti-IL-17F-01 antibody (as described above) and biotin-labeled anti-IL-17F-07 antibody (as described above) were used as capture and detection antibodies, respectively. For the detection of IL-17A/IL-17F heterodimers, an anti-IL-17A antibody (R&D Systems) was used as a capture antibody and biotin labeled anti-IL-17F-07 antibody was used as a detection antibody. The IL-17A and IL-17F antibodies are not cross-reactive (data not shown).

ELISAs were performed according to a well-known protocol. Briefly, ELISA plates were incubated with 2 μg/ml of capture antibody overnight. The plates were washed with PBS/1% BSA to remove excess capture antibody and incubated with serial dilutions of the conditioned media, (i.e., recombinant IL-17A/IL-17A, IL-17F/IL-17F, IL-17A/IL-17F cytokines) for 2 h at RT. After washing unbound cytokine, 0.07-0.5 μg/ml biotin-conjugated developing antibody was added and plates were incubated for 2 h at RT. Plates were washed to remove unbound developing antibody, saturating concentrations of avidin-horseradish peroxidase (HRP) were added, and plates were incubated for 1 h at RT. Unbound avidin-HRP was washed away using PBS/1% BSA. The assay was developed using TBM.

FIG. 11 demonstrates the detection of recombinant IL-17A and IL-17F homodimers, as well as the detection of recombinant IL-17A/IL-17F heterodimers. When capture and detection antibodies are both directed toward one cytokine, (i.e., IL-17A or IL-17F), both homodimers and heterodimers of that cytokine were detected (FIGS. 11A and 11B). In contrast, when an anti-IL-17A antibody and an anti-IL-17F antibody are used as capture and detection antibodies, respectively, only IL-17A/IL-17F heterodimers are detected (FIG. 11C). These data suggest that the IL-17A/IL-17F antibody pair may be used to detect and potentially purify natural (i.e., nonrecombinant) IL-17A/IL-17F heterodimers in conditioned media derived from primary cells. Additionally, these results suggest that anti-IL-17F-07 antibodies, and likely anti-IL-17F-01 antibodies and other anti-Il-17F antibodies, as described herein, may also be directed against IL-17A/IL-17F heterodimers.

### Example 5.2

### Detection of Natural IL-17A Homodimers, Natural IL-17F Homodimers, and Natural IL-17A/IL-17F Heterodimers

Human CD4⁺ T cells (5×10⁵ cells/ml) were activated with anti-CD3-coupled beads (5 μg/10⁷ beads), and increasing concentrations of anti-CD28 antibodies (R&D, Minneapolis, Minn.), and in the absence or presence of IL-21 (60 ng/ml) or IL-23 (0.5 ng/ml). Supernatants were collected 72 hours after primary activation and the concentration of IL-17A or IL-17F was determined by ELISA for IL-17A protein, or IL-17F protein, respectively, as described above (Example 5.1). FIG. 12 demonstrates that IL-21 or IL-23 may be used to enhance IL-17A or IL-17F production by T cells undergoing primary activation. IL-2, IL-7, and IL-15 also induced IL-17A and IL-17F production upon CD3/CD28 stimulation (data not shown).

Human CD4⁺ T cells (5×10⁵ cells/ml) were activated with anti-CD3-coupled beads (5 μg/10⁷ beads) and soluble anti-CD28 antibodies for 48 hours. Activated T cells were harvested, rested overnight, and reactivated in the presence of bead-bound anti-CD3, anti-CD28 antibodies, IL-21 (60 ng/ml) or IL-23 (0.5 ng/ml). Supernatants were collected 72 hours after secondary activation and production IL-17A or IL-17F was determined by ELISA for IL-17A protein, or IL-17F protein, respectively, as described above. FIG. 13 demonstrates that IL-21 or IL-23 synergizes with CD28 costimulation to enhance IL-17A or IL-17F production by T cells undergoing secondary activation.

Human CD4⁺ T cells (2×10⁶ cells/ml) were subjected to primary activation with anti-CD3-coupled beads (5 μg/10⁷ beads) and soluble anti-CD28 antibody (0.5 μg/ml). After 48 h of primary activation, conditioned media (CM1) was collected and cells rested overnight. The next day, cells were counted and restimulated at 2×10⁶ cells/ml as described for the primary activation above and in the presence of 60 ng/ml IL-21. After 72 h of restimulation, conditioned media (CM2) was collected. The presence or absence of IL-17A homodimers, IL-17F homodimers, and IL-17A/IL-17F heterodimers in neat and 1:10 diluted CM1 and CM2 was assessed using the ELISA formats described in Example 5.1.

The data indicate little to no detection of IL-17A homodimers or IL-17A/IL-17F heterodimers in CM1 media, i.e., media of T cells that underwent primary activation (FIGS. 14A and 14C). In contrast, conditioned media of restimulated T cells (CM2) comprised not only IL-17A and IL-17F homodimers but also IL-17A/IL-17F heterodimers (FIGS. 14A, 14B, and 14C). These data indicate that T cells, the “natural” source of IL-17 cytokines, express both homodimers and heterodimers of IL-17A and IL-17F. These results also indicate that antibodies directed against IL-17F can recognize and react with both IL-17F homodimers and IL-17A/IL-17F heterodimers, and that such antibodies may be used to isolate and inhibit the biological activity of IL-17F homodimers and/or IL-17A/IL-17F heterodimers.

### Example 5.3

### Immunoprecipitation of Natural IL-17A Homodimers, IL-17F Homodimers, and IL-17A/IL-17F Heterodimers from T Cells

Conditioned media (CM2) derived from T cells undergoing secondary activation in the presence of IL-21, as described in Example 5.2, was mixed with 20 μg/ml murine anti-human IL-17A-02 (Wyeth) or murine anti-human IL-17F-01 (Wyeth) monoclonal antibodies for 1 h at 4° C. under gentle rotation. Antibody complexes from each mixture were separately immunoprecipitated with 50 μl hydrated protein A-sepharose overnight at 4° C. under gentle rotation. The immunoprecipitated pellets were then sequentially washed with PBS/1% Tween 20, PBS/0.1% Tween 20, and PBS/0.05% Tween 20. The immunoprecipitated pellets were resuspended in nonreducing sample buffer and loaded onto a 10% Tricine gel for Western blot analysis with either anti-human IL-17A biotin conjugation (R&D, Minneapolis, Minn.) or rabbit anti-human IL-17F antibodies (Wyeth).

IL-17F homodimers (35 kDa) were immunoprecipitated using murine anti-human IL-17F-01 antibody and detected via Western blot analysis with a rabbit anti-human IL-17F polyclonal antibody from 500 μl of CM2. The IL-17F/IL-17A heterodimers (32 kDa) were immunoprecipitated using murine anti-human IL-17A-02 antibody and detected via Western blot analysis with a rabbit anti-human IL-17F polyclonal antibody from 500 μl of CM2 (FIG. 15). Neither the monoclonal nor the polyclonal antibody cross-reacted with IL-17A (data not shown). Similarly, IL-17A homodimers (31 kDa) and IL-17F/A heterodimers were immunoprecipitated using murine anti-human IL-17A-02 antibody and detected via Western blot analysis using a polyclonal goat anti-IL-17A biotinylated antibody from 700 μl of CM2. The IL-17F/IL-17A heterodimers were immunoprecipitated using murine anti-human IL-17F-01 antibody and detected via Western blot analysis using a polyclonal goat anti-IL-17A biotinylated antibody from 700 μl of CM2 (FIG. 16). The polyclonal antibody cross-reacts with IL-17F homodimer at high protein concentrations (data not shown).

As controls (lane 2 of FIGS. 15 and 16), IL-17F homodimers were purified from concentrated conditioned media overexpressing His tagged human IL-17F. Briefly, concentrated conditioned media was diluted 1:1 with 100 mM Tris pH 8/1M NaCl/10 mM imidazole and loaded onto a Nickel-NTA Fast Flow column (Qiagen, Valencia, Calif.). The homodimer was step eluted with 250 mM imidazole buffer. The protein was dialyzed against PBS-NSO. The homodimer was then digested with EK (enterokinase) for 4 hours at room temp to remove the His₆ tag. The digested protein was diluted 1:1 with 50 mM sodium phosphate pH 8/20 mM imidazole/300 mM NaCl and bound to a Nickel-NTA column (Qiagen, Valencia, Calif.). The protein minus the tag was eluted with 40 mM imidazole buffer, and was dialyzed against PBS NSO. Purified IL-17A homodimers for use as controls (lane 5 of FIG. 15) were purchased from R&D Systems (Minneapolis, Minn.).

### Example 5.4

### Immunoprecipitation of Recombinant IL-17A Homodimers, IL-17F Homodimers, and IL-17F/IL-17A Heterodimers from Transfected COS Cells

Experiments were conducted to determine whether recombinant human IL-17F and IL-17A would form heterodimers upon expression in COS cells, and whether anti-IL-17F and anti-IL-17A antibodies were capable of immunoprecipitating and detecting IL-17F/IL-17A heterodimers. COS cell cultures were transfected with IL-17F cDNA, IL-17A cDNA, or both IL-17F and IL-17A cDNA, and the transfected cell cultures were allowed to secrete the resultant protein(s) into the media. Conditioned media derived from either expression of IL-17F or IL-17A or the coexpression of IL-17F and IL-17A was mixed with 20 μg/ml murine anti-human IL-17A-02 (Wyeth) or murine anti-human IL-17F-01 (Wyeth) monoclonal antibodies for 1 hour at 4° C. under gentle rotation. Antibody complexes from each mixture were separately immunoprecipitated with 50 μl-hydrated protein A-sepharose overnight at 4° C. under gentle rotation. The immunoprecipitated pellets were then sequentially washed with PBS/1% Tween 20, PBS/0.1% Tween 20, and PBS/0.05% Tween 20. The immunoprecipitated pellets were resuspended in nonreducing sample buffer and loaded onto a 10% Tricine gel for Western blot analysis with either goat anti-human IL-17A (R&D, Minneapolis, Minn.) or rabbit anti-human IL-17F antibodies (Wyeth).

IL-17F homodimers (35 kDa) and IL-17F/IL-17A heterodimers were immunoprecipitated using murine anti-human IL-17F-01 antibody and detected via Western blot analysis with a rabbit anti-human IL-17F polyclonal antibody from 50 μl of CM2 (FIG. 17A; lanes 3 and 4, respectively). The IL-17F/IL-17A heterodimers (32 kDa) were also immunoprecipitated using murine anti-human IL-17A-02 antibody and detected via Western blot analysis with a rabbit anti-human IL-17F polyclonal antibody from 50 μl of CM2 (FIG. 17A; lane 9). The IL-17A antibody used for immunoprecipitation in lanes 8-10 of FIG. 17A appeared to cross-react with IL-17F at high protein concentrations, since a band corresponding to IL-17F homodimer is detected by the anti-IL-17F antibody probe in lane 10. As shown in FIG. 17B, the IL-17A homodimers (31 kDa) and IL-17F/IL-17A heterodimers were immunoprecipitated using murine anti-human IL-17A-02 antibody and detected via Western blot analysis using a polyclonal goat anti-human IL-17A antibody from 50 μl of CM2 (FIG. 17B; lanes 3 and 4, respectively). Also, the IL-17F/IL-17A heterodimers were immunoprecipitated using murine anti-human IL-17F-01 antibody and detected via Western blot analysis using a polyclonal goat anti-human IL-17A antibody from 50 μl of CM2 (FIG. 17B; lane 6). As opposed to the IL-17A antibody used for immunoprecipitation in FIG. 17A, the IL-17F antibody used for immunoprecipitation in lanes 5-7 of FIG. 17B did not significantly cross-react with IL-17A, since almost no band corresponding to IL-17A homodimer is detected by the anti-IL-17A antibody probe in lane 5.

As controls (lanes 6-7 of FIG. 17A), IL-17F homodimers were purified as described in Example 5.3. Purified IL-17A homodimers for use as controls (lane 5 of FIG. 17A) were purchased from R&D Systems (Minneapolis, Minn.). Control purified IL-17F homodimers migrate slightly faster than IL-17F homodimers immunoprecipitated from conditioned media. This is likely due to differences in glycosylation and/or the lack of an epitope tag on the IL-17F proteins in the purified samples.

### Example 5.5

### Purification of Recombinant IL-17A/IL-17F Heterodimers

Two methods of purifying IL-17A/IL-17F heterodimers are provided herein. In the first method, COS cells were cotransfected with His₆-tagged IL-17F (SEQ ID NO:36) and untagged IL-17A. Sodium chloride and imidazole were added to the conditioned media to final concentrations of 500 mM and 6 mM, respectively. The conditioned media was then loaded onto a Nickel NTA column (Qiagen, Valencia, Calif.). Thus, only IL-17F homodimer and IL-17F/IL-17A heterodimer, which contain a His tag, were captured on the nickel column. The IL-17F homodimer and IL-17F/IL-17A heterodimer were then separated with an imidazole gradient, and the IL-17F/IL-17A heterodimer was then digested with EK to remove the His₆ tag. The protein was dialyzed against PBS. Edman sequencing was done to verify that the IL-17F and IL-17A protein was detected in the IL-17F/A heterodimer sample. N-terminal sequence results confirmed the existence of heterodimers, i.e., the first five amino acids for IL-17F were shown to be RKIPK (SEQ ID NO:37), and for IL-17A were shown to be IVKAG (SEQ ID NO:38).

The second method used a dual column purification scheme, which is shown in the flow diagram set forth in FIG. 18. Flag-tagged human IL-17A (SEQ ID NO:39) and a His₆-tagged human IL-17F (SEQ ID NO:36) were cloned into separate pSMED2 vectors (Wyeth) and coexpressed in HEK293 cells by lipofection. Briefly, cells were seeded in two 175-cm² flasks 24 h prior to transfection. For each flask, 24 μg pSMED2/Flag-IL-17A+24 μg pSMED2/His6-IL-17F was mixed with 120 μl TRANSIT® reagent-LT1 (Mims, Madison, Wis.) in 2 ml serum-free media, and added to a flask containing cells (90% confluent) and 25 ml DMEM media containing 10% heat-inactivated fetal bovine serum. One day post-transfection, media was removed, the cells rinsed 1× with serum-free media, and fresh serum-free media was added (40 ml/flask). Conditioned media was harvested 48 h later, filtered (0.45 μL) to remove cells, and frozen at −20° C. Protein expression was confirmed by Western analysis using specific antibodies.

The conditioned media was batch-bound to anti-Flag M2 affinity resin (Sigma, St. Louis, Mo.) at 4° C. for 2 hours. The bound proteins, IL-17A homodimer and IL-17F/IL-17A heterodimer, were eluted using 50 mM Tris pH 8/500 mM NaCl/200 μg/ml Flag peptide (Sigma, St. Louis, Mo.). The flag elution was then batch-bound to Nickel-NTA resin (Qiagen, Valencia, Calif.) overnight at 4° C. The bound protein, IL-17F/IL-17A heterodimer, was eluted using 50 mM Tris pH 8/1 M NaCl/500 mM imidazole.

Following purification of recombinant IL-17F/IL-17A heterodimers substantially free from IL-17F and IL-17A homodimers, the activity of the heterodimers was tested on BJ cells by measuring the ability of the heterodimer to stimulate GRO-α levels in the BJ cell culture media. Thus, BJ cultures were stimulated with various concentrations of IL-17F homodimers, IL-17A homodimers, or the purified recombinant IL-17F/IL-17A heterodimers. After 24 h, supernatants from the BJ cultures were collected and GRO-α concentrations determined using commercially available ELISA. Briefly, BJ cells were seeded at 5×10³ cells/well in flat-bottom 96-well plates and supplied with 15 μl of media containing IL-17A homodimers, IL-17F homodimers, or IL-17F/IL-17A heterodimers. Plates were then incubated for 16-24 hours at 37° C., after which the supernatants were removed and the concentration of GRO-α determined using standard sandwich ELISA with antibodies to GRO-α (R & D Systems, Minneapolis, Minn.). Concentrations of GRO-α produced were determined based on a standard curve. The results are shown in FIG. 19A. Similar to both IL-17F and IL-17A homodimers, the IL-17A/IL-17F heterodimer is capable of stimulating IL-17 GRO-α concentrations in BJ cells. Moreover, as shown in FIG. 19B, treatment of BJ cultures with anti-IL-17A antibodies, or anti-IL-17A in combination with anti-IL-17F antibodies abrogated the ability of IL-17F/IL-17A heterodimers to stimulate GRO-α levels.

### Example 5.6

### Mass Spectrometry Analysis of IL-17A Homodimers, IL-17F Homodimers, and IL-17A/IL-17F Heterodimers

To provide direct evidence of a disulfide linkage between two IL-17 monomers, the presence of disulfide linkages was verified and intermolecular disulfide-linked peptides (IL-17A homodimers, IL-17F homodimers, and IL-17A/IL-17F heterodimers) were identified by tandem mass spectrometric analysis. Tryptic cleavage and reverse phase high performance liquid chromatography (rpHPLC) were used to isolate disulfide-linked peptides, which were then analyzed by nanoLC-MS/MS. Briefly, 1-2 ng of purified, nonreduced recombinant IL-17A homodimer, IL-17F homodimer and IL-17A/IL-17F heterodimer were run on 10% Bis-Tris gels (Invitrogen, Carlsbad, Calif.), and stained with the IMPERIAL™ Protein Stain Solution (Pierce, Rockford, Ill.). Positively stained bands were excised and manually trypsin digested (Promega, Madison, Wis.) for mass spectrometric analysis. For the digestion, the excised gel slices were dehydrated in acetonitrile (ACN), rehydrated and washed in 25 mM sodium phosphate buffer (pH 6.0), and dehydrated again in ACN. The protease trypsin (0.5 ng of trypsin dissolved in 25 mM sodium phosphate buffer) was added, driven into the gel pieces by rehydration, and incubated for 4 h at 37° C. The resulting peptides were further extracted from the gel with three successive washes using aliquots of 60% ACN/1% formic acid (FA) and 90% ACN/5% FA. These extracts were combined and evaporated, and the final sample reconstituted in 2% ACN and 0.1% FA.

The digested samples were pressure-loaded onto a C18 PICOFRIT® microcapillary column (New Objective, Woburn, Mass.) packed with Magic C18 beads (5 μm, 75 μm×11 cm, Michrom BioResources, Auburn, Calif.). The column was then coupled to a linear ion trap mass spectrometer (LTQ, ThermoFinnigan, San Jose, Calif.). The HPLC gradient increased linearly from 4-60% ACN using Solvent B as a modifier (Solvent A, 2% ACN and 0.1% FA; Solvent B, 90% ACN and 0.1% FA) over 70 min with a flow rate of 250 nl/min. Mass spectra were collected using LTQ at tandem mass spectrometry mode (referred to as MS/MS), in which each MS acquisition was followed by six MS/MS acquisitions of the first six most intense peptide ions in the prior MS spectrum. In some cases, MS/MS acquisitions were followed by two MS/MS/MS acquisitions of the first two most intense peptide ions of the prior MS/MS acquisitions. The peptide masses were recorded by scanning an m/z (mass-to-charge ratio) range from 375 to 1500. The dynamic exclusion in the acquisition software provided by the manufacturer was also employed to increase the number of peptide ions of interest to be analyzed. The MS/MS data were manually interpreted.

At the MS level, the observed m/z of the peptide fragments ([M+3H]³⁺=919.7 for IL-17A homodimer; [M+3H]³⁺=1196.9 for IL-17F homodimer; [M+3H]³⁺=1138.1 or 807.9 for IL-17F/IL-17A heterodimer) matched the calculated candidate disulfide-linked peptides. Further, the peptide sequence according to the MS/MS data of these targeted masses was obtained, and it was confirmed that they were the expected cysteine(C)-containing peptides derived from IL-17A and/or IL-17F for the disulfide bond formation as shown in FIG. 20. In addition, MS³ data was acquired for the IL-17A homodimer peptide ([M+3H]³⁺=907.7 and [M+3H]³⁺=843.3) due to the poor fragmentation at the MS/MS level. This demonstrates that IL-17A homodimers, IL-17F homodimers, and IL-17F/IL-17A heterodimers exist as dimers via disulfide bond linkage. In conclusion, the disulfide linkage patterns of the two homodimers and the heterodimer were resolved, which were consistent to be C1 and C4 between each monomer. Similar approaches were used in this study to demonstrate the involvement of other cysteines (C2/C3 and C5/C6) for the intra-molecular disulfide bond formation.

### Example 6

### Antibodies Against Human IL-17F Inhibit Primate IL-17F Bioactivity

To determine whether the anti-IL-17F antibodies were capable of cross-reacting with primate IL-17F, various concentrations of macaque IL-17F conditioned media or purified human IL-17F were used to stimulate BJ cells in the presence or absence of 100 μg/ml anti-IL-17F-01 or anti-IL-17F-07 antibodies. Macaque IL-17F conditioned media for use in this experiment was produced by expressing macaque IL-17F cDNA (SEQ ID NO:40) subcloned into pCRII in HEK293 cells, and harvesting the conditioned media containing the macaque IL-17F homodimers. After 16-24 hours of treatment with either human or macaque IL-17F, the GRO-α concentrations of supernatants collected from the treated cultures was determined using a commercially available ELISA (R&D, Minneapolis, Minn.) as described in Example 5.5. Concentrations of GRO-α were determined based on a standard curve.

FIG. 21 demonstrates increased GRO-α production by BJ cells incubated with macaque IL-17F conditioned media or human IL-17F protein. As described previously (see FIG. 2 and FIG. 8), human IL-17F stimulates GRO-α levels, and this stimulation is inhibited by both anti-IL-17F-01 and anti-IL-17F-07 antibodies (FIG. 21A). As shown in FIG. 21B, macaque IL-17F also stimulates production of GRO-α in BJ cells. Both anti-IL-17F-01 and anti-IL-17F-07 antibodies are capable of inhibiting the ability of macaque IL-17F conditioned media to stimulate GRO-α cytokine levels. Thus, antagonist antibodies to human IL-17F can cross-react with primate IL-17F to inhibit primate IL-17F bioactivity.

### Example 7

### IL-17F Stimulation of Aggrecanase 1 Levels in Human Chondrocytes is Reduced by Cotreatment with IL-17F Antibodies

To determine whether IL-17F may be involved in the inflammatory response accompanying, e.g., rheumatoid arthritis, nonarthritic human cartilage was obtained from National Disease Research Interchange (NDRI, Philadelphia, Pa.), and chondrocytes were isolated by serial enzymatic digestion of pronase (1 mg/ml, 37° C. for 30 min) and collagenase P (1 mg/ml, 37° C. overnight) within 48 hour postmortem. Upon isolation, cells were resuspended in Dulbecco's modified Eagle's medium (DMEM)/F-12 media with 2 μM L-glutamine, and 100 U/ml penicillin/100 μg/ml streptomycin containing 10% fetal bovine serum (FBS). Cells were plated in 24-well plates at 1×10⁶ cells/well. Media was changed to serum-free media after 72 h and the chondrocytes were stimulated with human IL-17F in the presence or absence of anti-IL-17F-07 or its isotype control (IgG) for 6 h at 37° C. Cells were harvested immediately in RLT buffer (Qiagen, Valencia, Calif., RNEASY® Kit) with β-mercaptoethanol and stored at −80° C. until ready for the RNA isolation. RNA was prepared using RNEASY® Mini Kit, and DNase treatment was performed on the RNEASY® column for 15 min at room temperature as per the manufacturer's protocol. ADAMTS-4 (aggrecanase-1) mRNA expression levels were monitored by TAQMAN® PCR analysis (Applied Biosystems, Foster City, Calif.). Briefly, 100 ng of isolated RNA was used for QPCR reaction with ADAMTS-4 probe/primers obtained from Applied Biosystems. The expression of ADAMTS-4 was normalized to the expression of GAPDH (10 ng of isolated RNA was used with GAPDH primers obtained from Applied Biosystems) for each sample. A standard curve for sample extrapolation was prepared using 0.16 ng to 100 ng of Universal Reference Total RNA (Clontech, Palo Alto, Calif.) that consists of pools of different tissues.

The results, presented as TAQMAN® units, are shown in FIG. 22. Thus, IL-17F treatment increases the expression of Aggrecanase 1 in human chondrocytes, and treatment with anti-IL-17F-07 antibodies abrogates this stimulation. These data suggest that IL-17F involvement in inflammation and joint degradation, such as occurs during autoimmune arthritis, e.g., reactive and rheumatoid arthritis, may be mitigated by treatment with anti-IL-17F antibodies.

### Example 8

### Regulation of IL-17F and IL-17A Bioactivity by siRNA Knockdown of Receptors IL-17R and IL-17RC in BJ Cells

Experiments were designed to determine whether a reduction in the level of IL-17R and/or IL-17RC transcripts would reduce the bioactivity of IL-17F and/or IL-17A. BJ cells were seeded 24 h prior to transfection in 96-well plates at 9×10³ cells/100 μl medium/well. Cells were transfected with chemically synthesized RNAi reagents (Dharmacon, Lafayette, Colo.) using DHARMAFECT® 1 (Dharmacon, Lafayette, Colo.) at 0.3 μl/well, and individual or pooled siRNAs at 10 nM or lower (see SEQ ID NOs:17-32). Following siRNA transfection, the cells were incubated with transfection complexes for 18 h. The transfection medium was then replaced by regular culture medium and incubated for an additional 6 h. The regular culture medium was then replaced with 150 μl of culture medium containing IL-17A at 1 ng/ml or IL-17F at 50 ng/ml. Following 16 h of incubation with the designated cytokine, the culture supernatants were collected for analysis by standard sandwich ELISA of the ability of IL-17F and/or IL-17A to induce levels of IL-6, IL-8, and GRO-α (see FIG. 2). Matched antibody pairs for hGRO-α, hIL-6 and hIL-8 were purchased from R&D Systems (Minneapolis, Minn.).

To measure decreases in IL-17R and IL-17RC receptor levels following siRNA treatment, the TURBOCAPTURE® mRNA kit (Qiagen, Valencia, Calif.) was used to isolate mRNA from BJ fibroblast cells according to manufacturer's instructions. A one-step RT qPCR MASTERMIX PLUS® (Eurogentec, San Diego, Calif.) TAQMAN® (Applied Biosystems) protocol was used whereby 10 μl of mRNA per sample was used in 25 μl TAQMAN® PCR reactions performed on an ABI PRISM® 7700 DNA Sequence Detector (Applied Biosystems). The conditions for TAQMAN® PCR were as follows: 30 min at 48° C., 10 min at 95° C., then 40 cycles each of 15 s at 95° C. and 1 min at 60° C. on MICROAMP OPTICAL® (Applied Biosystems) 96-well plates, covered with MICROAMP OPTICAL® caps. Each plate contained triplicates of the test cDNA templates or no-template controls for each reaction mix. The expression for each mouse gene was normalized to human (32 microglobulin gene expression. The TAQMAN® gene expression assay probe-primer sets for IL-17R and IL-17RC were acquired from Applied Biosystems.

The results presented in FIG. 23 depict the percent reduction in GRO-α levels (normalized to β2-microglobulin) following siRNA treatment of BJ cultures. siRNA treatment of BJ cultures decreased IL-17R and IL-17RC transcript levels by about 80% (FIG. 23A and FIG. 23B—“Taqman”). While decreases in both IL-17R and IL-17RC levels reduced the ability of IL-17A and IL-17F to stimulate GRO-α levels (FIG. 23A and FIG. 23B), reduction in IL-17RC levels (FIG. 23B) had a more pronounced effect than reduction in IL-17R levels (FIG. 23A) on both IL-17A and IL-17F bioactivity. Interestingly, the reduction in IL-17RC had a greater effect on the ability of IL-17A to stimulate GRO-α levels (FIG. 23B). Preferred examples of siRNAs that target IL-17R and IL-17RC are disclosed in FIG. 23C (see also SEQ ID NOs:17-32). These data suggest that both IL-17A and IL-17F can signal through IL-17R and IL-17RC, and that IL-17RC may be a preferred receptor for both molecules in relation to GRO-α stimulation.

### Example 9

### IL-17A and IL-17F are Upregulated in Afflicted Tissues from Human Patients with Psoriasis, Crohn's Disease, and Ulcerative Colitis

Psoriatic tissue biopsy samples were collected from patients enrolled in Wyeth-sponsored Clinical Study #3067K6-207 (A Randomized, Double-blind, Placebo-controlled, Exploratory Pharmacogenomic Study of Recombinant Human Interleukin Eleven, rhIL-11, in Patients with Active Psoriasis). Baseline (visit 2) psoriatic lesional and nonlesional skin biopsies from 48 patients were flash frozen in liquid nitrogen immediately after collection and shipped to the Wyeth Clinical Pharmacogenomics Laboratory in Andover, Mass.

Crohn's disease samples were collected from patients enrolled in Wyeth-sponsored Clinical Study #3067K6-204 (A Multicenter, Randomized, Double-blind, Placebo-controlled, Safety and Exploratory Pharmacogenomic Study of Orally Administered Recombinant Human Interleukin Eleven (RhIL-11) for the Treatment of Patients with Active Crohn's Disease), and ulcerative colitis samples were collected from patients enrolled in Clinical Study #3067K5-114 (A Multicenter, Randomized, Double-blind, Placebo-controlled, Dose-escalating, Safety and Exploratory Pharmacogenomic Study of Orally Administered Recombinant Human Interleukin Eleven (RhIL-11) in Patients with Mild to Moderate Left-sided Ulcerative Colitis). Baseline (visit 2) paired involved and noninvolved tissue biopsies from #3067K6-204 patients (16 patients), and baseline (visit 1) paired tissue biopsies from the same anatomical area of sigmoid and left colon from #3067K5-114 patients (12 patients) were flash frozen in liquid nitrogen immediately after collection and shipped to the Wyeth Clinical Pharmacogenomics Laboratory in Andover, Mass.

Tissue was homogenized using a polytron, RNA was isolated from the supernatant of the lysate using RNEASY® Mini Kit (Qiagen, Valencia, Calif.), and treated with DNase (Qiagen RNase-free DNase Kit). The DNase-treated RNA preparation was further purified using a Phase Lock Gel column (Brinkman, Westbury N.Y.), phenol:chloroform:IAA (isoamyl alcohol) extracted (Ambion, Austin, Tex.), and concentrated using an RNEASY® mini column. SPECTRAMAX® (Molecular Devices, Sunnyvale, Calif.) was used to quantify RNA, and RNA quality was assessed by Agilent Bioanalyzer gel (Model 2100; Agilent Technologies, Palo Alto, Calif.). Conversion of 2 μg of total RNA from the above preparations to cDNA was accomplished using the Applied Biosystems High Capacity cDNA Archive Kit (Applied Biosystems) by following the manufacturer's instructions. Plates containing completed reactions were stored at temperatures of −20° C. (short term) or −80° C. (long term).

Applied Biosystem's Assays-on-Demand (AOD) gene-specific primer-probe pairs are prevalidated, QC tested and optimized for use on any ABI PRISM® sequence detection system (all from Applied Biosystems). According to the manufacturer's AOD protocol, a master mix was prepared using TAQMAN® Universal PCR Master Mix (Applied Biosystems) containing IL-17F or IL-17A primers, and aliquoted into a 96-well plate for a final volume of 50 μl/well. Duplicate wells for serially diluted standards and cDNA samples (50 ng/well) were assayed on an ABI PRISM® 7700 Sequence detector (Sequence Detector Software v1.7) using universal thermal cycling conditions of 50° C. for 2 min, 95° C. for 10 min, 95° C. for 15 s (40 cycles), and extension at 60° C. for 1 min.

Relative quantification of RNA transcript levels of IL-17F and IL-17A was performed following the manufacturer's guidelines described for the ABI PRISM® 7700 Sequence Detection System (Applied Biosystems). Specifically, standard curves were calculated for target standards and endogenous control, input values determined for target and endogenous controls using standard curves' slope and y-intercept, and target input values were normalized to endogenous control. Fold-change in IL-17F and IL-17A expression was calculated using the 50 ng standard as a calibrator, and relative concentration of sample was obtained by multiplying fold-change by calibrator, then averaging.

To utilize the standard curve method, a tissue was empirically determined to express the target gene using TAQMAN® AOD (Applied Biosystems). Total RNA from over 10 candidate target-positive tissues was obtained from Wyeth (Cambridge/Andover) and outside vendors. Multiple 2 μg aliquots from a single RNA preparation were converted to cDNA (as described above), pooled, stored as aliquots at −80° C., and assayed for expression of target gene by TAQMAN® (Applied Biosystems). Cycle threshold (Ct) values of ≧35 were considered below the limits of detection. For standard curve development, the goal was to achieve a Ct value between 18 and 25 for 100 ng of cDNA; this allowed for appropriate standard curve dynamic range. Preparations of positive control tissue meeting these requirements were used to generate the standard curve for each assay. Standard curves consisted of two-fold serial dilutions of total cDNA from 100 ng/well to 1.5 ng/well. Standard curves were performed on each plate for every assay and were used for sample quantification and assay performance monitoring. Due to 96-well plate space constraints, standard curve dilution points of 25 ng and 3 ng were omitted when running samples. Inter-plate % CV was <3% for psoriasis samples, and <5% for Crohn's disease and ulcerative colitis samples.

Genes that are expressed at similar levels in all samples (i.e., treated and untreated, lesional and nonlesional, etc.) were selected to serve as endogenous controls in the relative standard curve method. From a list of candidate endogenous controls, it was determined that the gene designated ZNF592 (GenBank Accession No. NM 014630) produced acceptable standard curves and did not vary significantly in lesional and nonlesional tissues, and involved and noninvolved tissues (p<0.05 for psoriasis and p<0.09 for Crohn's disease and ulcerative colitis samples). All study samples were normalized to ZNF592 levels in determining relative concentration values.

A paired Student's t-test (pairing lesional and nonlesional samples, or involved and noninvolved tissues, from each patient) was used to assess the significance of the association between IL-17F and IL-17A expression levels and lesional (psoriasis) or inflammatory phenotype (Crohn's disease or ulcerative colitis). Fold-changes for the psoriasis study were calculated by dividing lesional relative concentration values by nonlesional relative concentration values. Fold-changes for the Crohn's disease and ulcerative colitis (IBDs) studies were obtained by dividing involved relative concentration values by noninvolved relative concentration values. Summary fold-changes were calculated by averaging fold-changes from all patients for IL-17F of IL-17A levels.

The results of these studies are shown in FIG. 24 and FIG. 25. As shown in FIG. 24, both IL-17F and IL-17A expression levels are significantly increased in lesional tissues from afflicted patients, suggesting that IL-17F and IL-17A are involved in psoriasis in vivo. As shown in FIG. 25, both IL-17A and IL-17F are increased in the involved tissues from patients afflicted with Crohn's disease, as well as those from patients afflicted with ulcerative colitis. The considerable heterogeneity among patients with Crohn's disease and ulcerative colitis, coupled with the relatively small sample size, mitigated against identifying a statistically significant association of IL-17A and IL-17F with the involved phenotype. However, clustering tools showed that both IL-17A and IL-17F were well correlated with the involved phenotype in the Crohn's disease sample set (r=0.65) (data not shown). These data suggest that elevated levels of IL-17A and IL-17F in involved tissues may play a role in the inflammatory conditions associated with IBDs in vivo.

### Example 10

### LN Cells from Ovalbumin Immunized Mice Produce IL-17F

8-week-old C57BL/6 mice were immunized in the flanks with 100 ovalbumin protein emulsified in complete Freund's adjuvant. Seven days later, inguinal lymph nodes were harvested. Lymph nodes were dissociated and the cells were restimulated with 50 ng phorbol ester 12-tetradecanoylphorbol-13 acetate, 1 μg/mlionomycin, and 1 μg/ml GOLGIPLUG™ for 12 hours. Cells were then harvested, stained for surface CD4 using anti-mouse CD4 PerCP Cy5.5 (Pharmagen, San Diego, Calif.). Cells were fixed and permeabilized with CYTOFIX/CYTOPERM™ (BD Biosciences, San. Diego, Calif.) after which cells were stained with 4 μg/ml rat IgG1 ALEXA FLUOR® 647 conjugate (Invitrogen, Carlsbad, Calif.) or with 4 μg/ml rat anti-IL-17F (clone 15-1) ALEXA FLUOR® 647 (Invitrogen, Carlsbad, Calif.) for 30 min. Cells were then washed twice with PERM/WASH™ (BD Biosciences, San. Diego, Calif.), and analyzed using FACSCALIBUR™ (BD Biosciences, San. Diego, Calif.). Rat anti-IL-17F (clone 15-1) ALEXA FLUOR® 647 (Invitrogen, Carlsbad, Calif.) was prepared using an ALEXA FLUOR® 647 conjugation kit from Invitrogen. The results are shown in FIG. 26. Thus, in vivo CD4+ T cells from the lymph nodes of ovalbumin-immunized mice produce IL-17F protein.

### Example 11

### Conclusion and Discussion

Among several findings, these data indicate that IL-21 and IL-23 induced IL-17A and IL-17F upon TCR/CD28 costimulation, and that IL-23 and IL-21 synergize with costimulation for IL-17A and IL-17F production. Both IL-23 and IL-21 are equally effective in IL-17 induction. These data suggest that IL-17A, and particularly IL-17F (since it is produced at 10-20 fold higher levels compared to IL-17A) may mediate some of the proinflammatory effects attributed to IL-21, and that inhibition of IL-17F (either as an IL-17F homodimer or an IL-17F heterodimer) may have similar therapeutic effects as blocking IL-21 signaling (see, e.g., U.S. Patent Application Nos. 60/599,086 and 60/639,176). The similarities between the effects of IL-17F signaling and IL-21 signaling lead to a strong conclusion that inhibition of IL-17F signaling may be as therapeutically valuable as inhibiting IL-21 signaling. Additionally, the results show for the first time that T cells express IL-17A/IL-17F heterodimers, as well as IL-17A and IL-17F homodimers; the results also show that such cytokines may be isolated and purified in their natural and recombinant forms. The data presented herein also shows that anti-IL-17F antibodies, fusion proteins comprised of IL-17F, and siRNA targeting IL-17R and IL-17RC reduce IL-17F bioactivity. Further, the results show that IL-17F treatment increases Aggrecanase expression in human chondrocytes, which can be reduced by anti-IL-17F antibodies, and that IL-17F and IL-17A are elevated in psoriatic lesions and tissues involved in IBD from human biopsies.

IL-17A and IL-17F are novel proinflammatory cytokines produced by activated T cells. These cytokines share a high degree of amino acid identity, including conserved cysteines that exhibit structural features of a cysteine knot motif. Both cytokines have been proposed to share receptor chains and exhibit similar biological functions. Members of the IL-17 cytokine family have been implicated in diseases mediated by abnormal immune responses such as rheumatoid arthritis, inflammatory bowel disorders (IBDs) and asthma. Due to the similarities enumerated above, IL-17A and IL-17F produced by human T cells upon activation were characterized. CD4+ T cells were activated with anti-CD3 in the presence or absence of CD28 costimulation, γ-common cytokines (IL-2, IL-4, IL-7, IL-15, IL-21) or IL-23. Optimal production of IL-17A and IL-17F required TCR as well as CD28 costimulation. Additionally, CD28 and IL-21 act synergistically in IL-17A and IL-17F production, suggesting IL-17A and IL-17F may mediate proinflammatory effects attributed to IL-21 signaling. Under all activating conditions, protein levels of IL-17F were 10-20 fold above those obtained for IL-17A. Interestingly, in addition to IL-17A homodimers and IL-17F homodimers, T cells also produced IL-17A/IL-17F heterodimers. These findings suggest that multiple forms of these cytokines are present in vivo, with each form accounting for distinct biological functions, e.g., that the IL-17A/IL-17F heterodimer may constitute a new cytokine target in the treatment of inflammatory diseases.

1-7. (canceled)

8. A method of treating a subject at risk for, or diagnosed with, a disorder related to increased IL-17F signaling comprising administering to the subject a therapeutically effective amount of an IL-17F signaling antagonist.

9. The method of claim 8, wherein the IL-17F signaling antagonist is selected from the group consisting of IL-17F inhibitory polynucleotides, IL-17R inhibitory polynucleotides, IL-17RC inhibitory polynucleotides, soluble polypeptides comprising IL-17R or IL-17F binding fragments thereof, soluble polypeptides comprising IL-17RC or IL-17F binding fragments thereof, inhibitory anti-IL-17F antibodies, inhibitory anti-IL-17R antibodies, inhibitory IL-17RC antibodies, and antagonistic small molecules.

10. The method of claim 9, wherein the IL-17F signaling antagonist is an IL-17R inhibitory polynucleotide.

11. The method 0 f claim 9, wherein the IL-17F signaling antagonist is an IL-17RC inhibitory polynucleotide.

12. The method of claim 10, wherein the inhibitory polynucleotide is an siRNA selected from the group consisting of the nucleotide sequences set forth in SEQ ID NOs:17-24.

13. The method of claim 11, wherein the inhibitory polynucleotide is an siRNA selected from the group consisting of the nucleotide sequences set forth in SEQ ID NOs:25-32.

14. The method of claim 9, wherein the IL-17F signaling antagonist is a soluble polypeptide comprising IL-17R or IL-17F binding fragments thereof.

15. The method of claim 9, wherein the IL-17F signaling antagonist is a soluble polypeptide comprising IL-17RC or IL-17F binding fragments thereof.

16. The method of claim 14, wherein the soluble polypeptide has the amino acid sequence set forth in SEQ ID NO:34.

17. The method of claim 15, wherein the soluble polypeptide has the amino acid sequence set forth in SEQ ID NO:35.

18. The method of claim 9, wherein the IL-17F inhibitory polynucleotide comprises the nucleotide sequence set forth in, or a nucleotide sequence complementary to the nucleotide sequence set forth in, SEQ ID NO:1 or a fragment of SEQ ID NO:I, or an RNA equivalent thereof, and wherein expression of the inhibitory polynucleotide in a cell results in the decreased expression of IL-17F.

19. The method of claim 9, wherein the IL-17R inhibitory polynucleotide comprises the nucleotide sequence set forth in, or a nucleotide sequence complementary to the nucleotide sequence set forth in, SEQ ID NO:5 or a fragment of SEQ ID NO:5, or an RNA equivalent thereof, and wherein expression of the inhibitory polynucleotide in a cell results in the decreased expression of IL-17R.

20. The method of claim 9, wherein the IL-17RC inhibitory polynucleotide comprises a nucleotide sequence selected from the group consisting of the nucleotide sequences set forth in, or a nucleotide sequence complementary to a nucleotide sequence selected from the group consisting of the nucleotide sequences set forth in, SEQ ID NO:7, SEQ ID NO:9, SEQ ID NO:II, SEQ ID NO: 13, and SEQ ID NO: 15 or a fragment of a nucleotide sequence selected from the group consisting of the nucleotide sequences set forth in SEQ ID NO:7, SEQ ID NO:9, SEQ ID NO:II, SEQ ID NO:13, and SEQ ID NO:15, or an RNA equivalent thereof, and wherein expression of the inhibitory polynucleotide in a cell results in the decreased expression of IL-17RC.

21. The method of claim 8, wherein the disorder related to increased IL-17F signaling is an inflammatory disorder.

22. The method of claim 21, wherein the inflammatory disorder is selected from the group consisting of an autoimmune disease, a respiratory disease, and an inflammatory bowel disease.

23. The method of claim 22, wherein the inflammatory disorder is an autoimmune disease, and the autoimmune disease is selected from the group consisting of arthritis, psoriasis, systemic lupus erythematosus, and multiple sclerosis.

24. The method of claim 23, wherein the autoimmune disease is rheumatoid arthritis.

25. The method of claim 22, wherein the inflammatory disorder is a respiratory disease, and the respiratory disease is cystic fibrosis.

26. The method of claim 22, wherein the inflammatory disorder is an inflammatory bowel disease.

27. The method of claim 8, further comprising administering to the subject a therapeutically effective amount of at least one additional therapeutic agent.

28. The method of claim 27, wherein the at least one additional therapeutic agent is selected from the group consisting of cytokine inhibitors, growth factor inhibitors, immunosuppressants, anti-inflammatory agents, metabolic inhibitors, enzyme inhibitors, cytotoxic agents, and cytostatic agents.

29. The method of claim 27, wherein the at least one additional therapeutic agent is selected from the group consisting of TNF antagonists, anti-TNF agents, IL-12 antagonists, IL-15 antagonists, IL-17 antagonists, IL-18 antagonists, IL-22 antagonists, T cell-depleting agents, B cell-depleting agents, cyclosporin, FK-506, CCI-779, etanercept, infliximab, rituximab, adalimumab, prednisolone, azathioprine, gold, sulphasalazine, chloroquine, hydroxychloroquine, minocycline, anakinra, abatacept, methotrexate, leflunomide, rapamycin, rapamycin analogs, Cox-2 inhibitors, cPLA2 inhibitors, NSAIDs, p38 inhibitors, antagonists of B7.1, B7.2, ICaSL, Icas and/or CD28, and agonists of CTLA4.

30-75. (canceled)

76. The method of claim 22, wherein the inflammatory disorder is a respiratory disease, and the respiratory disease is asthma.


SEQUENCE LISTING (40 sequences)

SEQ ID NO: 1 - DNA, 492 bp, Homo sapiens
  CDS (1)..(492)
atgacagtgaagaccctgcatggcccagccatggtcaagtacttgctgctgtcgatattggggcttgcctttctgagtgaggcggcagctcggaaaatccccaaagtaggacatacttttttccaaaagcctgagagttgcccgcctgtgccaggaggtagtatgaagcttgacattggcatcatcaatgaaaaccagcgcgtttccatgtcacgtaacatcgagagccgctccacctccccctggaattacactgtcacttgggaccccaaccggtacccctcggaagttgtacaggcccagtgtaggaacttgggctgcatcaatgctcaaggaaaggaagacatctccatgaattccgttcccatccagcaagagaccctggtcgtccggaggaagcaccaaggctgctctgtttctttccagttggagaaggtgctggtgactgttggctgcacctgcgtcacccctgtcatccaccatgtgcagtaa

SEQ ID NO: 2 - protein, 163 aa, Homo sapiens
MTVKTLHGPAMVKYLLLSILGLAFLSEAAARKIPKVGHTFFQKPESCPPVPGGSMKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ

SEQ ID NO: 3 - DNA, 1883 bp, Homo sapiens
  CDS (54)..(521)
gaattccggcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaatgaactttaagtatgagaaaagttcagcccaagtaaaataaaaactcaatcacattcaattccagagtagtttcaagtttcacatcgtaaccattttcgcccggaattcaaaaaaaaa

SEQ ID NO: 4 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 5 - DNA, 3420 bp, Homo sapiens
  CDS (134)..(2734)
ggctggaagccggaagcgagcaaagtggagccgactcgaactccaccggcacgagggcggaaaagaaagcctcagaacgttcgctcgctgcgtccccagccggggccgagccctccgcgacgccacccgggccatgggggccgcacgcagcccgccgtccgctgtcccggggcccctgctggggctgctcctgctgctcctgggcgtgctggccccgggtggcgcctccctgcgactcctggaccaccgggcgctggtctgctcccagccggggctaaactgcacggtcaagaatagtacctgcctggatgacagctggattcaccctcgaaacctgaccccctcctccccaaaggacctgcagatccagctgcactttgcccacacccaacaaggagacctgttccccgtggctcacatcgaatggacactgcagacagacgccagcatcctgtacctcgagggtgcagagttatctgtcctgcagctgaacaccaatgaacgtttgtgcgtcaggtttgagtttctgtccaaactgaggcatcaccacaggcggtggcgttttaccttcagccactttgtggttgaccctgaccaggaatatgaggtgaccgttcaccacctgcccaagcccatccctgatggggacccaaaccaccagtccaagaatttccttgtgcctgactgtgagcacgccaggatgaaggtaaccacgccatgcatgagctcaggcagcctgtgggaccccaacatcaccgtggagaccctggaggcccaccagctgcgtgtgagcttcaccctgtggaacgaatctacccattaccagatcctgctgaccagttttccgcacatggagaaccacagttgctttgagcacatgcaccacatacctgcgcccagaccagaagagttccaccagcgatccaacgtcacactcactctacgcaaccttaaagggtgctgtcgccaccaagtgcagatccagcccttcttcagcagctgcctcaatgactgcctcagacactccgcgactgtttcctgcccagaaatgccagacactccagaaccaattccggactacatgcccctgtgggtgtactggttcatcacgggcatctccatcctgctggtgggctccgtcatcctgctcatcgtctgcatgacctggaggctagctgggcctggaagtgaaaaatacagtgatgacaccaaatacaccgatggcctgcctgcggctgacctgatccccccaccgctgaagcccaggaaggtctggatcatctactcagccgaccaccccctctacgtggacgtggtcctgaaattcgcccagttcctgctcaccgcctgcggcacggaagtggccctggacctgctggaagagcaggccatctcggaggcaggagtcatgacctgggtgggccgtcagaagcaggagatggtggagagcaactctaagatcatcgtcctgtgctcccgcggcacgcgcgccaagtggcaggcgctcctgggccggggggcgcctgtgcggctgcgctgcgaccacggaaagcccgtgggggacctgttcactgcagccatgaacatgatcctcccggacttcaagaggccagcctgcttcggcacctacgtagtctgctacttcagcgaggtcagctgtgacggcgacgtccccgacctgttcggcgcggcgccgcggtacccgctcatggacaggttcgaggaggtgtacttccgcatccaggacctggagatgttccagccgggccgcatgcaccgcgtaggggagctgtcgggggacaactacctgcggagcccgggcggcaggcagctccgcgccgccctggacaggttccgggactggcaggtccgctgtcccgactggttcgaatgtgagaacctctactcagcagatgaccaggatgccccgtccctggacgaagaggtgtttgaggagccactgctgcctccgggaaccggcatcgtgaagcgggcgcccctggtgcgcgagcctggctcccaggcctgcctggccatagacccgctggtcggggaggaaggaggagcagcagtggcaaagctggaacctcacctgcagccccggggtcagccagcgccgcagcccctccacaccctggtgctcgccgcagaggagggggccctggtggccgcggtggagcctgggcccctggctgacggtgccgcagtccggctggcactggcgggggagggcgaggcctgcccgctgctgggcagcccgggcgctgggcgaaatagcgtcctcttcctccccgtggaccccgaggactcgccccttggcagcagcacccccatggcgtctcctgacctccttccagaggacgtgagggagcacctcgaaggcttgatgctctcgctcttcgagcagagtctgagctgccaggcccaggggggctgcagtagacccgccatggtcctcacagacccacacacgccctacgaggaggagcagcggcagtcagtgcagtctgaccagggctacatctccaggagctccccgcagccccccgagggactcacggaaatggaggaagaggaggaagaggagcaggacccagggaagccggccctgccactctctcccgaggacctggagagcctgaggagcctccagcggcagctgcttttccgccagctgcagaagaactcgggctgggacacgatggggtcagagtcagaggggcccagtgcatgagggcggctccccagggaccgcccagatcccagctttgagagaggagtgtgtgtgcacgtattcatctgtgtgtacatgtctgcatgtgtatatgttcgtgtgtgaaatgtaggctttaaaatgtaaatgtctggattttaatcccaggcatccctcctaacttttctttgtgcagcggtctggttatcgtctatccccaggggaatccacacagcccgctcccaggagctaatggtagagcgtccttgaggctccattattcgttcattcagcatttattgtgcacctactatgtggcgggcatttgggataccaagataaattgcatgcggcatggccccagccatgaaggaacttaaccgctagtgccgaggacacgttaaacgaacaggatgggccgggcacggtggctcacgcctgtaatcccagcacactgggaggccgaggcaggtggatcactctgaggtcaggagtttgagccagcctggccaacatggtgaaaccccatctccactaaaaatagaaaaattagccgggcatggtgacacatgcctgtagtcctagctacttgggaggctgaggcaggagaattgcttgaatctgggaggcagaggttgcagtgagccgagattgtgccattgcactgcagcctggatgacagagcgagactctatctcaaaaaaaaa

SEQ ID NO: 6 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 7 - DNA, 2691 bp, Homo sapiens
  CDS (219)..(2594)
aaaacgaaagcactccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccggtgagtctggaaccctggggagacgaggaaaggctcagggttcagtttttggctcagcaaagccttagcctggctcctgtcactgctgccactgccagaactgccctgtctggtctgtctggtgctgatggtagaagagaagaacggggaaggggcaagagctgggtctgtctttctctgggagggtctgggaatacggagccccagaaaaagggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctacccatgtggcccaaaaaaaaaaaaaaaaaaaaaaaaaaaaaaaaaaaaaaaaaaaaaaaaaaaaaaaaaaaaa

SEQ ID NO: 8 - protein, 791 aa, Homo sapiens
MPVPWFLLSLALGRSPVVLSLERLVGPQDATHCSPVSLEPWGDEERLRVQFLAQQSLSLAPVTAATARTALSGLSGADGRREERGRGKSWVCLSLGGSGNTEPQKKGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 9 - DNA, 2478 bp, Homo sapiens
  CDS (219)..(2381)
aaaacgaaagcactccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctacccatgtggcccaaaaaaaaaaaaaaaaaaaaaaaaaaaaaaaaaaaaaaaaaaaaaaaaaaaaaaaaaaaaa

SEQ ID NO: 10 - protein, 720 aa, Homo sapiens
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 11 - DNA, 2527 bp, Homo sapiens
  CDS (219)..(1835)
aaaacgaaagcactccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcggggggtgagtgggagcaagcgctgggcggagggccgcccccggggagccaggcctgtgccagctcacctcttccctccccatctgttttctccggcagcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctacccatgtggcccaaaaaaaaaaaaaaaaaaaaaaaaaaaaaaaaaaaaaaaaaaaaaaaaaaaaaaaaaaaaa

SEQ ID NO: 12 - protein, 538 aa, Homo sapiens
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGGEWEQALGGGPPPGSQACASSPLPSPSVFSGSGRQGPRGSAPLLSR

SEQ ID NO: 13 - DNA, 2584 bp, Homo sapiens
  CDS (219)..(1022)
aaaacgaaagcactccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctggtgaggcctcccccttcccaagtccattcccactgtaggccgatgcctgtgcaaaggacgcagtgccatatcagagaggatccttgaagaggactcaccccaagcaagggaaaattggtgggggaacttctgccttcctggtttccttgactttggcctcctcctcttcctccttatcttctccaacctccttcctttatttgttccacagactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctacccatgtggcccaaaaaaaaaaaaaaaaaaaaaaaaaaaaaaaaaaaaaaaaaaaaaaaaaaaaaaaaaaaaa

SEQ ID NO: 14 - protein, 267 aa, Homo sapiens
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPALPWLNVSADGDNVHLVLNVSEEQHFGLSLYWNQVQGPPKPRWHKNLVRPPPSQVHSHCRPMPVQRTQCHIREDP

SEQ ID NO: 15 - DNA, 2427 bp, Homo sapiens
  CDS (219)..(494)
aaaacgaaagcactccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctggggtgccaccaaattctgggcttggaacagcttcagctcccacccgctcctccacacacagacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctggtgaggcctcccccttcccaagtccattcccactgtaggccgatgcctgtgcaaaggacgcagtgccatatcagagaggatccttgaagaggactcaccccaagcaagggaaaattgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctacccatgtggcccaaaaaaaaaaaaaaaaaaaaaaaaaaaaaaaaaaaaaaaaaaaaaaaaaaaaaaaaaaaaa

SEQ ID NO: 16 - protein, 91 aa, Homo sapiens
MPVPWFLLSLALGRSPVVLSLERLVGPQDATHCSPGLSCRLWGATKFWAWNSFSSHPLLHTQTVTYSACLGTSCLLRAPCWRLRTCRQSWC

SEQ ID NO: 17 - RNA, 21 nt, Artificial
  siRNA IL-17R
gaacaccaaugaacguuuguu

SEQ ID NO: 18 - RNA, 21 nt, Artificial
  siRNA IL-17R
caaacguucauugguguucuu

SEQ ID NO: 19 - RNA, 21 nt, Artificial
  siRNA IL-17R
gcaccuacguagucugcuauu

SEQ ID NO: 20 - RNA, 21 nt, Artificial
  siRNA IL-17R
uagcagacuacguaggugcuu

SEQ ID NO: 21 - RNA, 21 nt, Artificial
  siRNA IL-17R
cagaaccaauuccggacuauu

SEQ ID NO: 22 - RNA, 21 nt, Artificial
  siRNA IL-17R
uaguccggaauugguucuguu

SEQ ID NO: 23 - RNA, 21 nt, Artificial
  siRNA IL-17R
aauaugaggugaccguucauu

SEQ ID NO: 24 - RNA, 21 nt, Artificial
  siRNA IL-17R
ugaacggucaccucauauuuu

SEQ ID NO: 25 - RNA, 21 nt, Artificial
  siRNA IL-17RC
guacgaaucugguccuauauu

SEQ ID NO: 26 - RNA, 21 nt, Artificial
  siRNA IL-17RC
uauaggaccagauucguacuu

SEQ ID NO: 27 - RNA, 21 nt, Artificial
  siRNA IL-17RC
gaaccugacuccguuaggauu

SEQ ID NO: 28 - RNA, 21 nt, Artificial
  siRNA IL-17RC
uccuaacggagucagguucuu

SEQ ID NO: 29 - RNA, 21 nt, Artificial
  siRNA IL-17RC
gcuaugggacgaugacuuguu

SEQ ID NO: 30 - RNA, 21 nt, Artificial
  siRNA IL-17RC
caagucaucgucccauagcuu

SEQ ID NO: 31 - RNA, 21 nt, Artificial
  siRNA IL-17RC
gaccgcagaucauuaccuuuu

SEQ ID NO: 32 - RNA, 21 nt, Artificial
  siRNA IL-17RC
aagguaaugaucugcggucuu

SEQ ID NO: 33 - protein, 21 aa, Apis mellifera
MKFLVNVALVFMVVYISYIYA

SEQ ID NO: 34 - protein, 527 aa, artificial
  fusion protein human IL-17R - human IgG1
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWGSGSGSGEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 660 aa, artificial
  Fusion protein human IL-17RC - human IgG1
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRAGSGSGSGEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKS

SEQ ID NO: 36 - DNA, 510 bp, artificial
  His-tagged human IL-17F
atgaaattcttagtcaacgttgcccttgtttttatggtcgtgtacatttcttacatctatgcgggatctggtcaccaccatcatcaccacggtgacgatgacgataagcggaaaatccccaaagtaggacatacttttttccaaaagcctgagagttgcccgcctgtgccaggaggtagtatgaagcttgacattggcatcatcaatgaaaaccagcgcgtttccatgtcacgtaacatcgagagccgctccacctccccctggaattacactgtcacttgggaccccaaccggtacccctcggaagttgtacaggcccagtgtaggaacttgggctgcatcaatgctcaaggaaaggaagacatctccatgaattccgttcccatccagcaagagaccctggtcgtccggaggaagcaccaaggctgctctgtttctttccagttggagaaggtgctggtgactgttggctgcacctgcgtcacccctgtcatccaccatgtgcagtaa

SEQ ID NO: 37 - protein, 5 aa, Homo sapiens
RKIPK

SEQ ID NO: 38 - protein, 5 aa, homo sapiens
IVKAG

SEQ ID NO: 39 - DNA, 507 bp, artificial
  Flag-tagged human IL-17A
atgaaattcttagtcaacgttgcccttgtttttatggtcgtgtacatttcttacatctatgcgggatctcctgactataaagacgatgacgataagatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaa

SEQ ID NO: 40 - DNA, 492 bp, Macaca sp.
atgacagtgaagaccctgcatggcccagtcatggtcaagtacttgctgctgttgatattgggacttgcctttctgaatgaggtggcagctaggaaaatccccaaagtaggacatacttttttccaaaagcctgagagttgcccacctgtgccagaaggtagtatgaagcttgacactggcatcatcaatgaaaaccagcgtgtttccatgtcacgtaacatcgagagccgctccacctccccctggaattacactgtcacttgggaccccaaccggtatccctcggaagttgtacaggcccagtgtaagcacttgggctgcatcaatgctcaaggaaaggaagacatctccatgaattccgttcccatccagcaagagaccctggtcctccggaggaagcaccaaggctgctctgtttctttccagttggagaaggtgctggtgactgttggctgcacctgcgtcacccccgtcgtccaccatgtgcagtaa